(19) United States
(12) Patent Application Publication
(10) Pub. No.: US 2021/0130849 A1
(43) Pub. Date: May 6, 2021

(54) HUMAN GENE CORRECTION

(71) Applicant: Oregon Health & Science University, Portland, OR (US)

(72) Inventors: Shoukhrat Mitalipov, Beaverton, OR (US); Nuria Marti-Gutierrez, Portland, OR (US)

(73) Assignee: Oregon Health & Science University, Portland, OR (US)

(21) Appl. No.: 16/605,770

(22) PCT Filed: Apr. 20, 2018
(86) PCT No.: PCT/US2018/028560

§ 371 Date: Oct. 16, 2019

Related U.S. Application Data
(60) Provisional application No. 62/487,989, filed on Apr. 20, 2017.

(51) Int. Cl. C12N 15/90; C12N 15/873; C12N 5/073; A01K 67/027

(52) U.S. Cl. CPC C12N 15/907; C12N 15/873; C12N 5/0604; A01K 2227/106; C12N 2800/80; C12N 2310/20; A01K 67/027

(57) ABSTRACT

Methods are disclosed for correcting a mutant allele of a gene of interest in a primate cell. The methods include a) introducing a non-naturally occurring targeted nuclease and site-specific nucleotide-binding guide that act together to introduce double-stranded breaks in the mutant allele into the primate cell, wherein: i) the primate cell is undergoing mitotic cell division; ii) the primate cell comprises a genome that is heterozygous for the mutant allele, such that the genome comprises one copy of the mutant allele and one copy of a wild-type allele; iii) single-stranded oligonucleotides homologous to the wild-type allele are not introduced into the primate cell. The methods also include b) allowing the primate cell to activate homology-directed repair of the double-stranded DNA breaks in the mutant allele, thereby correcting the mutant allele using the normal wild-type allele as a repair template and producing a primate cell that is homozygous for the wild-type allele. The primate cell can be a one-cell embryo and/or a human cell.

### CROSS REFERENCE TO RELATED APPLICATION(S)

This claims the benefit of U.S. Application No. 62/487,989, filed Apr. 20, 2017, which is incorporated by reference herein.

### FIELD OF THE DISCLOSURE

This relates to the field of gene correction, specifically to the use of a non-naturally occurring targeted nuclease and site-specific nucleotide-binding guide that, together, introduce double-stranded breaks in mutant alleles present in a heterozygous primate cell, thereby correcting the mutant allele using the normal wild-type allele as a repair template and producing a primate cell (such as, but not limited to, a one-cell embryo) that is homozygous for the wild-type allele.

### BACKGROUND

More than 10,000 monogenic inherited disorders have been identified, affecting millions of people worldwide. Among these are the autosomal dominant mutations, where inheritance of a single copy of a defective gene can result in clinical symptoms. Dominant mutations that manifest as late-onset adult disorders include BRCA1 and BRCA2, which are associated with a high risk of breast and ovarian cancers (Antoniou, et al., Am J Hum Genet, 72:1117-1130, 2003), and MYBPC3, which causes hypertrophic cardiomyopathy (HCM; Carrier et al., Gene, 573:188-197, 2015). Because of delayed manifestation, these mutations are not naturally selected against but are often transmitted to the next generation. Consequently, the frequency for some of these founder mutations in particular human populations is very high. For example, the MYBPC3 mutation carrying a 25-bp deletion in major Indian populations is found at frequencies ranging from 2% to 8% (Dhandapany et al., Nat Genet, 41:187-191, 2009), while the estimated frequency of both BRCA1 and BRCA2 mutations among Ashkenazi Jews exceeds 2% (Struewing, et al., N Engl J Med, 336:1401-1408, 1997). There is a need for correcting these mutations in primate cells, such as primate somatic cells and one-cell embryos.

HCM is a myocardial disease characterized by left ventricular hypertrophy, myofibrillar disarray, and myocardial stiffness. There is an estimated prevalence of 1:500 in adults (Maron, et al., Circulation, 92:785-789, 1995) that manifests clinically with heart failure and sudden death. MYBPC3 mutations are the most frequent genetic cause of HCM and constitute a large part of other inherited cardiomyopathies (Schlossarek, et al., J Mol Cell Cardiol, 50:613-620, 2011). MYBPC3 encodes for the thick filament associated protein, cardiac myosin-binding protein C (cMyBP-C), a signaling node in cardiac myocytes that contributes to the maintenance of sarcomeric structure as well as regulation of both contraction and relaxation (Carrier et al., Gene, 573:188-197, 2015).

Current treatment options for HCM provide mostly symptomatic relief without addressing the genetic cause of the disease. One approach for preventing second-generation transmission is preimplantation genetic diagnosis (PGD) followed by selection of non-mutant embryos for transfer in the context of an in vitro fertilization (IVF) cycle. When only one parent carries a heterozygous mutation, 50% of the embryos should be mutation-free and available for transfer. However, discarding 50% of carrier embryos obviously impacts pregnancy rates and generates ethical dilemmas for families Thus, development of novel strategies preventing germline transmission of founder mutations is desirable, such as for disease caused by MYBPC3. These methods are also applicable to the correction of other founder mutations and the treatment of disorders caused by mutations in these genes. Furthermore, these strategies also can be applied in somatic cells.

### SUMMARY OF THE DISCLOSURE

Methods are disclosed herein for correcting a mutant allele of a gene of interest in a primate cell. These method include step a), introducing a non-naturally occurring targeted nuclease and site-specific nucleotide-binding guide that work together to introduce double-stranded breaks in the mutant allele into the primate cell, wherein: i) the primate cell is undergoing mitotic cell division; ii) the primate cell includes a genome that is heterozygous for the mutant allele, such that the genome includes one copy of the mutant allele and one copy of a wild-type allele; and iii) single-stranded oligonucleotides homologous to the wild-type allele are not introduced into the primate cell. The targeted nuclease can be clustered regularly interspaced short palindromic repeats (CRISPR)-associated (Cas)9, zinc finger protein (ZNF), or transcription activator-like effectors (TALEN). The method also includes step b), allowing the primate cell to activate homology-directed repair of the double-stranded DNA breaks in the mutant allele, thereby correcting the mutant allele using the normal wild-type allele as a repair template and producing a primate cell that is homozygous for the wild-type allele. In some embodiments, the primate is a human. In some embodiments, the primate cell is an embryonic cell, such as, but not limited to, a one-cell embryo.

The foregoing and other features and advantages of the invention will become more apparent from the following detailed description of several embodiments that proceeds with reference to the accompanying figures.

### BRIEF DESCRIPTION OF THE FIGURES

FIG. 1. Gene correction in S-phase-injected human embryos. A schematic of MYBPC3^{ΔGAGT} gene targeting by injection of the CRISPR/Cas9 into human zygotes at the S-phase of cell cycle. MII oocytes were fertilized by sperm from the heterozygous patient having equal numbers of mutant and WT spermatozoa. CRISPR/Cas9 was then injected into one-cell zygotes. Embryos at 4-8-cell stages were collected for genetic analyses.

FIGS. 2A-2F. Gene-targeting and homology-directed repair (HDR) efficiency in S-phase-injected human embryos. FIG. 2A, gene-targeting efficiency in zygote, S-phase-injected embryos. FIG. 2B, blastomere genotyping outcomes in mosaic embryos. FIG. 2C, distribution of various blastomere genotypes in mosaic embryos. FIG. 2D, overall targeting and HDR efficiencies. FIG. 2E, targeting efficiency in patient iPSCs and S-phase-injected embryos. FIG. 2F, yield of WT/WT embryos in control (n=19) and S-phase-injected (n=54) embryos.

FIGS. 3A-3E. Gene correction in M-phase-injected human embryos. FIG. 3A, a schematic of MYBPC3^{ΔGAGT} gene targeting by injecting CRISPR/Cas9 into M-phase oocytes. The CRISPR/Cas9 was mixed with a sperm suspension and co-injected into MII oocytes during ICSI. The M-phase delivery of the CRISPR/Cas9 allows genome editing to occur when a sperm contains a single mutant copy and, thus, produces uniform embryos and eliminates mosaicism. FIG. 3B, targeting efficiency in M-phase-injected embryos. FIG. 3C, yield of WT/WT embryos in control (n=19) and M-phase-injected (n=58) embryos. FIG. 3D, HDR outcomes in the presence or absence of ssODN. FIG. 3E, estimated HDR efficiencies in S-phase- and M-phase-injected embryos in comparison to untreated controls.

FIGS. 4A-4D. Digenome-seq based off-target mutation screening of treated human embryos. FIG. 4A, Genome-wide Circos plots showing DNA cleavage scores. Cas9 only-treated DNA is shown in grey, and the CRISPR/Cas9-treated DNA is shown in blue. FIG. 4B, a sequence logo obtained via WebLogo using Digenome-captured sites (DNA cleavage score >2.5). An on-target sequence is indicated below the sequence logo (SEQ ID NO: 6). FIG. 4C, on-target indels for 28 individual blastomeres detected by Digenome-seq. Only blastomeres carrying NHEJ signature were captured by Digenome-seq. FIG. 4D, indel frequencies for potential off-target sites captured by Digenome-seq in CRISPR/Cas9-treated (n=5) and untreated control human embryos (n=2; SEQ ID NOS: 7-30, top to bottom). Mismatch nucleotides are underlined. OnT: on target site; OT: off-target site.

FIGS. 5A-5F. CRISPR/Cas9 design and testing in patient iPSCs. FIG. 5A, and FIG. 5B, schematics of CRISPR/Cas9-1 (SEQ ID NO: 2) and CRISPR/Cas9-2 (SEQ ID NO: 3) constructs (sequences in the wild-type and mutant alleles, SEQ ID NOS: 31 and 32, respectively).

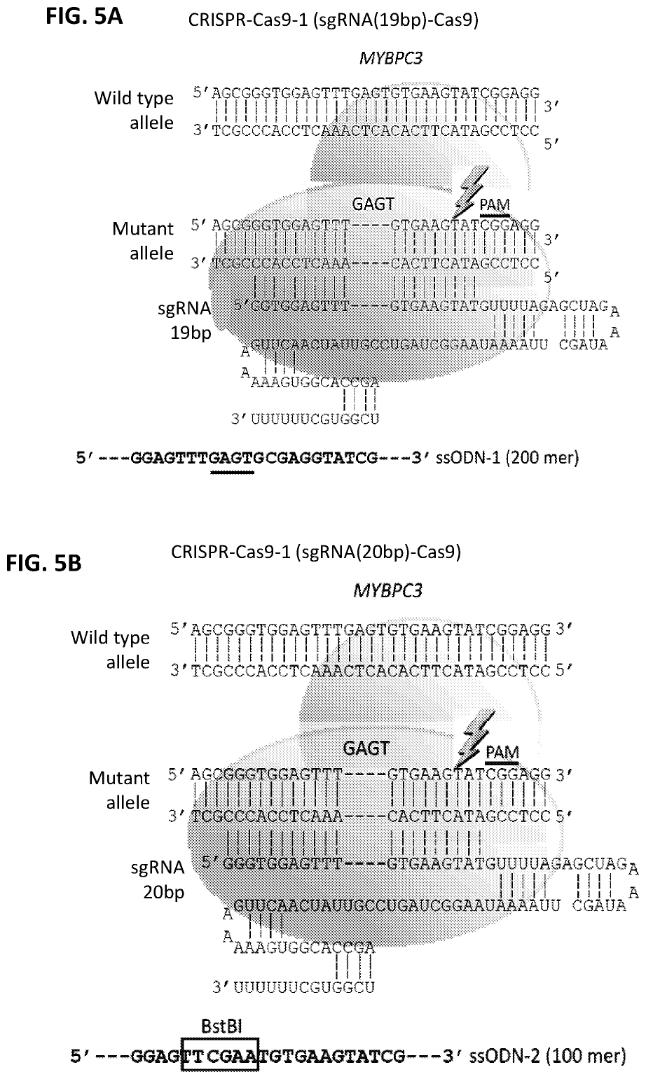

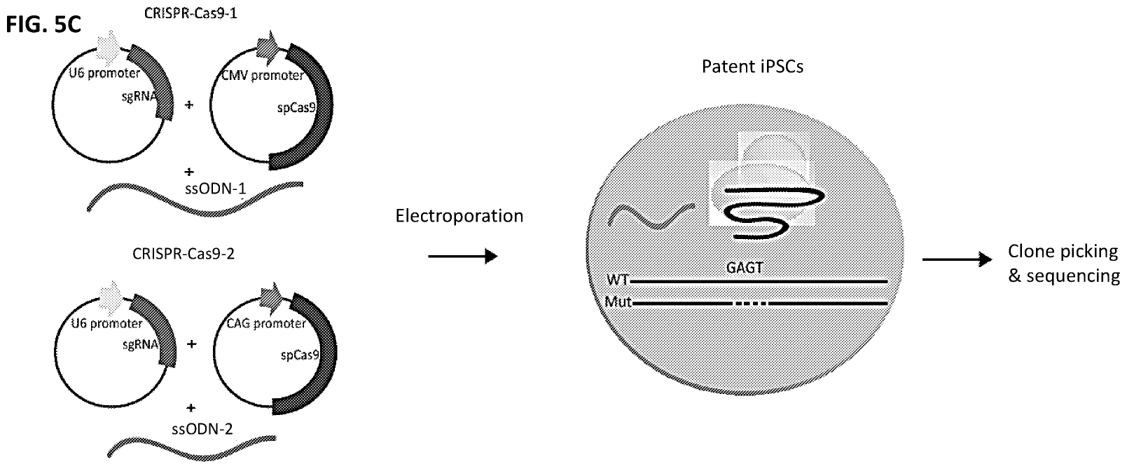

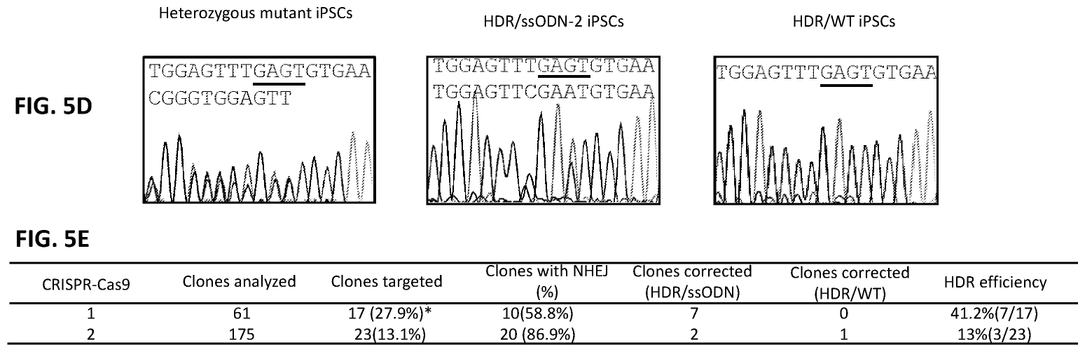

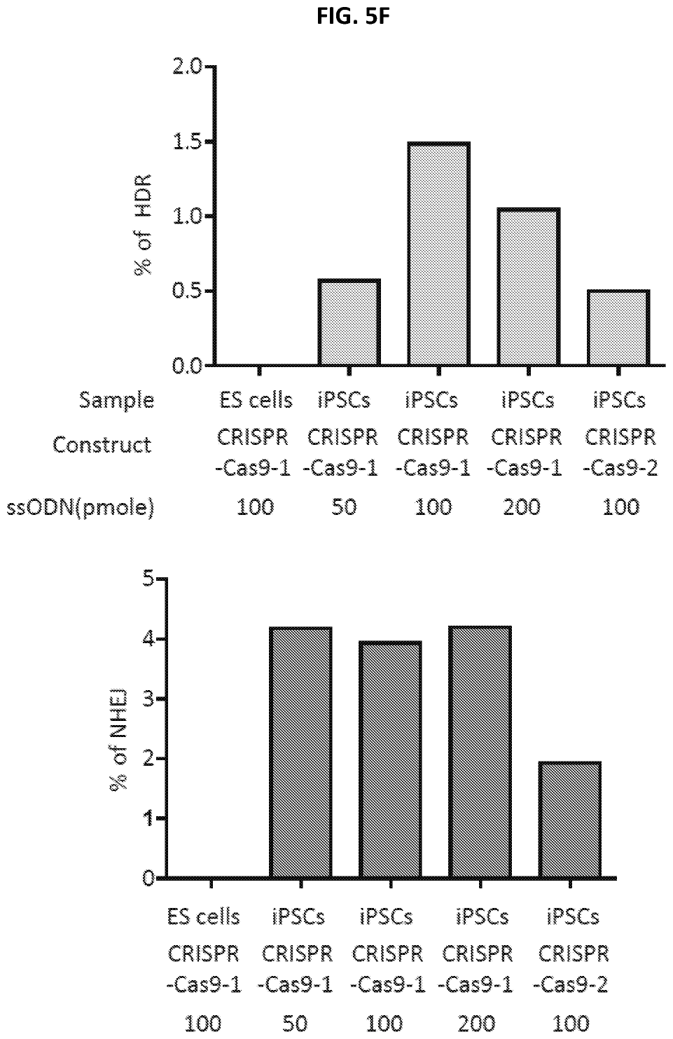

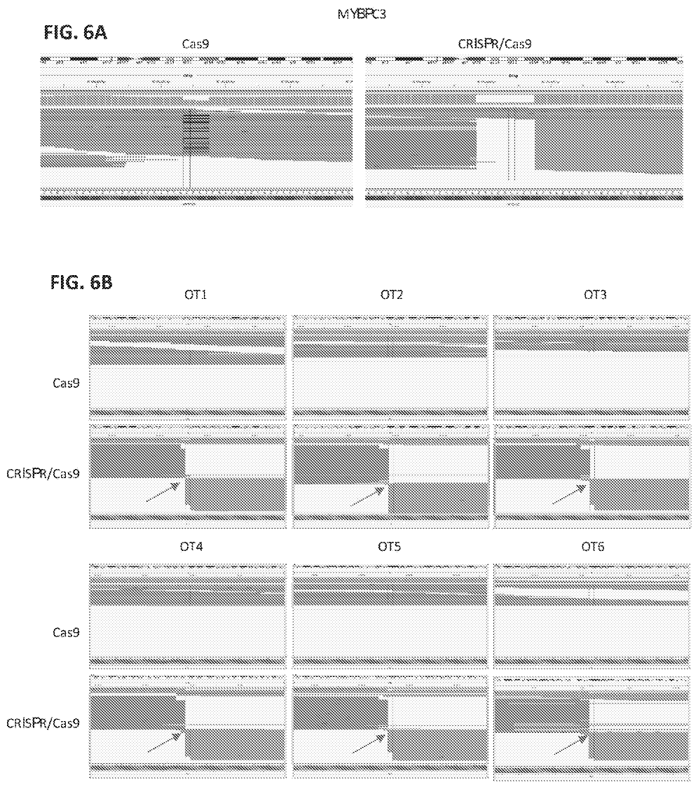

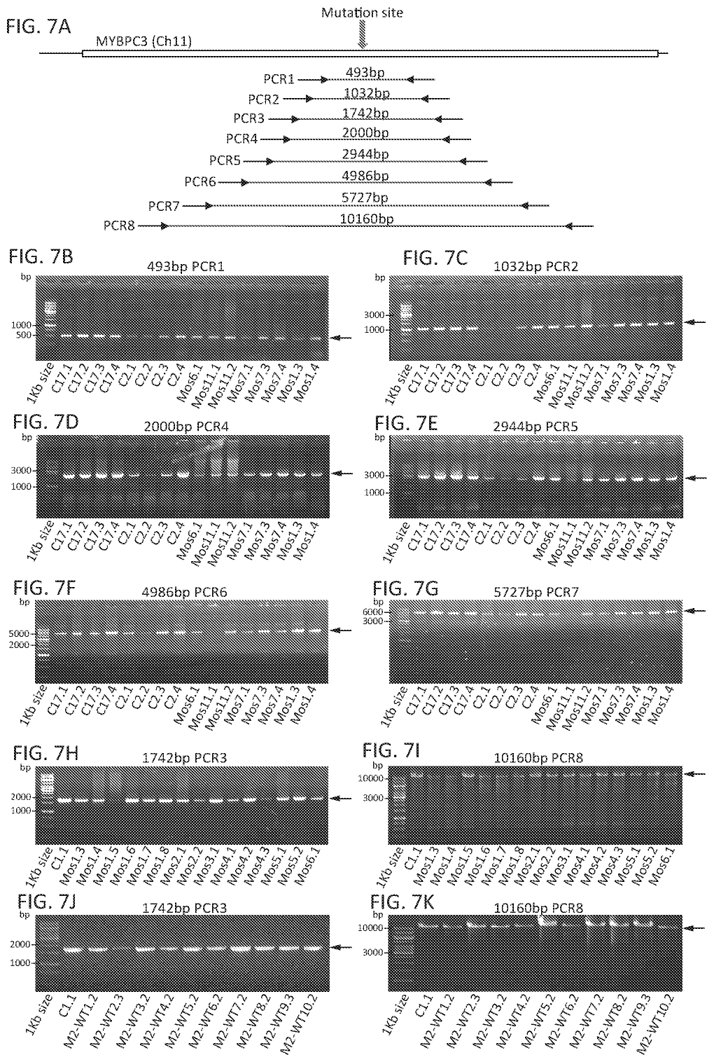

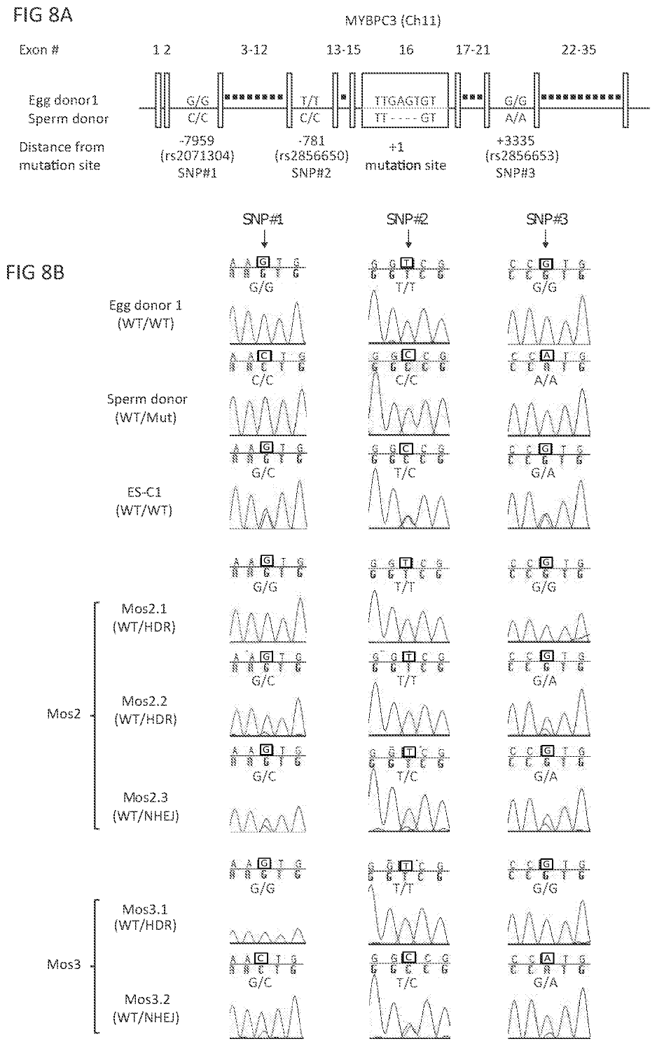

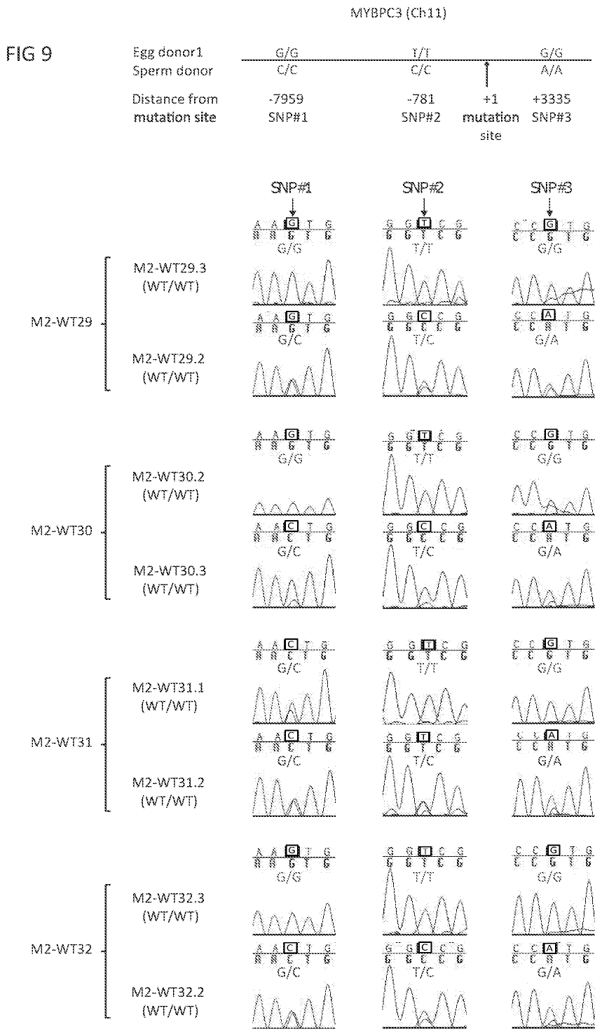

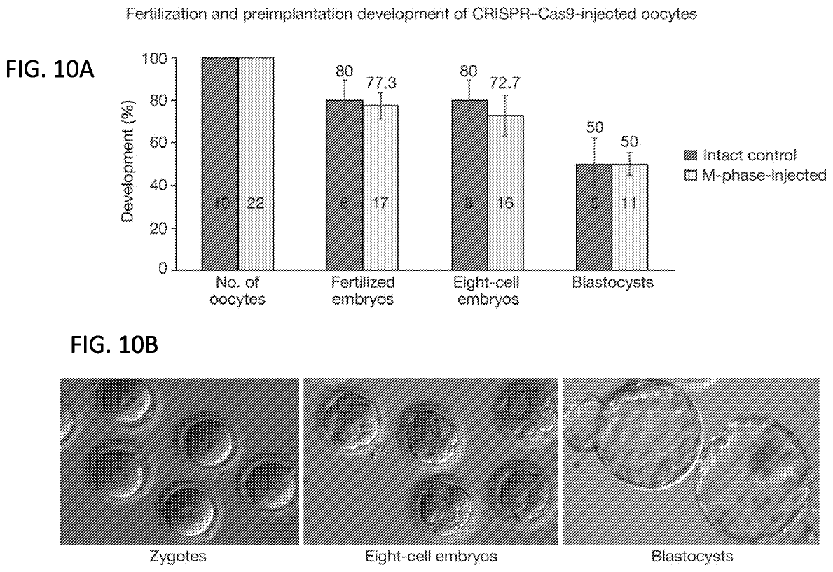

Both systems consist of a single-chain chimeric sgRNA designed to target the MYBPC3^{ΔGAGT} deletion and Cas9 protein. Exogenous single-stranded oligodeoxynucleotide (ssODN) templates encoding homology arms to the targeted region were designed for each system for facilitating HDR (ssODN-1, SEQ ID NO: 33; ssODN-2, SEQ ID NO: 34). Synonymous single nucleotide substitutions were introduced into each ssODN template, as indicated by underlining. In addition, the ssODN-2 nucleotide substitutions provide a restriction enzyme (BstBI) recognition site as indicated with an open box. FIG. 5C, patient iPSCs were transfected with CRISPR/Cas9 plasmids by electroporation, and individual single iPSC cloned were analyzed. FIG. 5D, representative chromatographs showing an untargeted iPSC clone with heterozygous mutant (left; SEQ ID NOS: 35 and 36, top to bottom), a targeted iPSC clone with gene corrected via HDR using ssODN-2 as a repair template (middle SEQ ID NOS: 37 and 38, top to bottom), and a targeted iPSC clone with gene corrected via HDR using the WT sequence as a template (SEQ ID NO: 39). FIG. 5E, a targeting and HDR efficiency comparison between CRISPR/Cas9-1 and CRISPR/Cas9-2. FIG. 5F, HDR and NHEJ efficiency in wild-type ES cells (H9) and patient iPSCs transfected with preassembled Cas9 ribonucleoproteins (RNPs).

FIGS. 6A-6B. Digenome sequencing of potential off-target sites. FIG. 6A, representative IGV (Integrative Genomics Viewer) images produced using the CRISPR/Cas9 at the on-target site. Mismatched nucleotides are shown in the lighter grey. FIG. 6B, representative IGV images showing the CRISPR/Cas9-induced DNA cleavage at the potential off-target sites. Arrows indicate DNA cleavage sites at each off-target site.

FIGS. 7A-7K. Long-range PCR analysis for detection of large deletions in individual blastomeres of mosaic and M-phase-injected human embryos. FIG. 7A, a schematic of 8 long-range PCR primers spanning the MYBPC3^{ΔGAGT} mutation site. FIGS. 7B-7G, agarose gel images of PCR1, PCR2, and PCR4-PCR7 amplifications in CRISPR-Cas9-targeted and control blastomeres. FIGS. 7H-7I, representative agarose gel images of PCR3 and PCR8 in CRISPR-Cas9-targeted and control blastomeres. FIGS. 7J-7K, representative agarose gel images of PCR3 and PCR8 in M-phase-injected WT/WT and control blastomeres. Arrows denote PCR bands that reflect the expected DNA size.

FIGS. 8A-8B. Evaluation of HDR repair and conversion tract length in mosaic and control human embryos produced from egg donor 1. FIG. 8A, a schematic map of 3 informative single nucleotide polymorphisms (SNPs) within a genomic region of the MYBPC3 gene (wild-type, top; mutant, bottom). The rs number under each SNP represents a reference number recorded at NCBI dbSNP (the Short Genetic Variation database). FIG. 8B, representative chromatographs of SNP genotypes in individual blastomeres from mosaic and control embryos.

FIG. 9. Evaluation of HDR repair and conversion tract length in S-phase and M-phase-injected WT/WT human embryos. Representative chromatographs of SNP genotypes in individual blastomeres from S-phase and M-phase-injected WT/WT human embryos.

FIGS. 10A-10B. Preimplantation development of CRISPR-Cas9-injected embryos. FIG. 10A, fertilization of CRISPR-Cas9-treated (n=22) and control (n=10) MII oocytes and their subsequent development to the eight-cell and blastocyst stage embryos. The numbers of oocytes/embryos/blastocysts are shown in bars; the percentages are shown above the bars. The error bars are the mean±s.e.m. Significance was established using the Student's t-test. FIG. 10B, representative images showing normal morphology of CRISPR-Cas9-injected pronuclear stage zygotes, eight-cell embryos, and blastocysts.

### SEQUENCE LISTING

The nucleic and amino acid sequences listed in the accompanying sequence listing are shown using standard letter abbreviations for nucleotide bases and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Apr. 20, 2018, 18.5 KB, which is incorporated by reference herein. In the accompanying sequence listing:

SEQ ID NO: 1 is an exemplary Streptococcus pyogenes Cas9 amino acid sequence.

SEQ ID NO: 2 is an exemplary gRNA target nucleic acid sequence.

SEQ ID NO: 3 is an exemplary gRNA target nucleic acid sequence.

SEQ ID NO: 4 is an exemplary gRNA nucleic acid sequence.

SEQ ID NO: 5 is an exemplary gRNA nucleic acid sequence.

SEQ ID NO: 6 is an exemplary on-target MYBPC3 mutant nucleic acid sequence.

SEQ ID NO: 7 is an exemplary human RPS14 nucleic acid sequence.

SEQ ID NO: 8 is an exemplary human intergenic nucleic acid sequence.

SEQ ID NO: 9 is an exemplary human intergenic nucleic acid sequence.

SEQ ID NO: 10 is an exemplary human TTC7B nucleic acid sequence.

SEQ ID NO: 11 is an exemplary human SLC36A2 nucleic acid sequence.

SEQ ID NO: 12 is an exemplary human HS6ST3 nucleic acid sequence.

SEQ ID NO: 13 is an exemplary human intergenic nucleic acid sequence.

SEQ ID NO: 14 is an exemplary human intergenic nucleic acid sequence.

SEQ ID NO: 15 is an exemplary human MRP22 nucleic acid sequence.

SEQ ID NO: 16 is an exemplary human intergenic nucleic acid sequence.

SEQ ID NO: 17 is an exemplary human intergenic nucleic acid sequence.

SEQ ID NO: 18 is an exemplary human intergenic nucleic acid sequence.

SEQ ID NO: 19 is an exemplary human XRRA1 nucleic acid sequence.

SEQ ID NO: 20 is an exemplary human intergenic nucleic acid sequence.

SEQ ID NO: 21 is an exemplary human SHROOM4 nucleic acid sequence.

SEQ ID NO: 22 is an exemplary human CDS2 nucleic acid sequence.

SEQ ID NO: 23 is an exemplary human RP11-718G2.5 nucleic acid sequence.

SEQ ID NO: 24 is an exemplary human MPP6 nucleic acid sequence.

SEQ ID NO: 25 is an exemplary human AUTS2 nucleic acid sequence.

SEQ ID NO: 26 is an exemplary human DECR1 nucleic acid sequence.

SEQ ID NO: 27 is an exemplary human intergenic nucleic acid sequence.

SEQ ID NO: 28 is an exemplary human NAA16 nucleic acid sequence.

SEQ ID NO: 29 is an exemplary human NR6A1 nucleic acid sequence.

SEQ ID NO: 30 is an exemplary human MYBPC3 nucleic acid sequence.

SEQ ID NO: 31 is an exemplary wild-type MYBPC3 nucleic acid sequence.

SEQ ID NO: 32 is an exemplary mutant MYBPC3 nucleic acid sequence.

SEQ ID NO: 33 is an exemplary single-stranded oligo donor (ssODN) nucleic acid sequence.

SEQ ID NO: 34 is an exemplary single-stranded oligo donor (ssODN) nucleic acid sequence.

SEQ ID NO: 35 is an exemplary MYBPC3 heterozygous nucleic acid sequence.

SEQ ID NO: 36 is an exemplary MYBPC3 heterozygous nucleic acid sequence.

SEQ ID NO: 37 is an exemplary ssODN-corrected MYBPC3 heterozygous nucleic acid sequence.

SEQ ID NO: 38 is an exemplary ssODN-corrected MYBPC3 heterozygous nucleic acid sequence.

SEQ ID NO: 39 is an exemplary wild-type MYBPC3 nucleic acid sequence.

SEQ ID NO: 40 is an exemplary primer nucleic acid sequence.

SEQ ID NO: 41 is an exemplary primer nucleic acid sequence.

SEQ ID NO: 42 is an exemplary adaptor nucleic acid sequence.

SEQ ID NO: 43 is an exemplary adaptor nucleic acid sequence.

### DETAILED DESCRIPTION OF SEVERAL EMBODIMENTS

Genome editing carries potential for the targeted correction of germline mutations. In the disclosed methods, a non-naturally occurring targeted nuclease and site-specific nucleotide-binding guide was used to induce double-strand breaks at the mutant paternal allele; these breaks were then predominantly repaired in the embryo using the homologous wild-type maternal gene instead of a synthetic DNA template. By modulating the cell cycle, mosaicism was avoided, resulting in a high yield of homozygous cells (such as, but not limited to, embryos) carrying the wild-type gene and without evidence of off-target mutations.

The disclosed methods have sufficient efficiency, accuracy, and safety, such that they are be suitable for correction of heritable mutations in human embryos. Thus, germline gene correction using the disclosed methods represents an alternative to preimplantation genetic diagnosis and has the advantage of rescuing mutant embryos. An exemplary CRISPR/Cas9-based correction of the heterozygous mutation in human preimplantation embryos is disclosed. The methods induce correction with precise targeting accuracy and dramatically high homology-directed repair (HDR) efficiency by activating an endogenous, germline-specific DNA repair response.

### Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GENBANK® accession nos. referred to herein are the sequences available on Apr. 20, 2017. All references, patent applications and publications, and GENBANK® accession nos. cited herein are incorporated by reference. To facilitate review of the various embodiments of the disclosure, the following explanations of specific terms are provided.

Animal: Living multi-cellular vertebrate organisms; a category that includes, for example, mammals, and birds. The term mammal includes both human and non-human mammals. Similarly, the term “subject” includes both human and veterinary subjects.

Allele: A different form of a specific gene. Mammals have two sets of chromosomes and, thus, are diploid and have homologous chromosomes. If both alleles at a gene (or locus) on the homologous chromosomes are the same, they and the organism are homozygous with respect to that gene (or locus). If the alleles are different, they and the organism are heterozygous with respect to that gene. The term “wild-type” allele is used to describe an allele that contributes to the typical phenotypic character in a normal (healthy) organism. A “variant” or “mutant” allele is usually recessive or dominant, less frequent in a population, and deleterious to the organism, such as causing a disease.

Cell culture: Cells grown under controlled conditions. A primary cell culture is a culture of cells, tissues, or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Clustered regularly interspaced short palindromic repeats (CRISPR)-associated protein 9 (Cas9): An RNA-guided DNA endonuclease enzyme associated with the CRISPR (clustered regularly interspersed palindromic repeats) adaptive immunity system in Streptococcus pyogenes, among other bacteria. Cas9 can cleave nearly any sequence complementary to the guide RNA. Includes Cas9 nucleic acid molecules and proteins. Cas9 sequences are publically available, for example from the GENBANK® sequence database (e.g., accession nos. NP_269215.1 and AKS40378.1 provide exemplary Cas9 protein sequences, while accession no. NC_002737.2 provides an exemplary Cas9 nucleic acid sequence therein, all incorporated by reference). One of ordinary skill in the art can identify additional Cas9 nucleic acid and protein sequences, including Cas9 variants.

Donor polynucleotide: A polynucleotide that is capable of specifically inserting into a genomic locus.

Double-strand breaks (in DNA): Breaks in which both strands of the double helix are severed. Three mechanisms are available for repair of double-strand breaks: non-homologous end joining (NHEJ), microhomology-mediated end joining (MMEJ), and homologous recombination (HR, an example of homology-directed repair).

Downstream: A relative position on a polynucleotide, wherein the “downstream” position is closer to the 3′ end of the polynucleotide than the reference point. In the instance of a double-stranded polynucleotide, the orientation of 5′ and 3′ ends are based on the sense strand, as opposed to the antisense strand.

Embryo: A cellular mass obtained by one or more divisions of a zygote without regard to whether it has been implanted into a female. A “one-cell” embryo is a single cell produced by the fusion of a maternal genome in an egg and a paternal genome from a sperm. A “morula” is the preimplantation embryo 3-4 days after fertilization, when it is a solid mass, generally composed of 12-32 cells (blastomeres). A “blastocyst” refers to a preimplantation embryo in placental mammals (about 3 days after fertilization in the mouse and about 5 days after fertilization in humans) of about 30-150 cells. The blastocyst stage follows the morula stage and can be distinguished by its unique morphology. The blastocyst is generally a sphere made up of a layer of cells (the trophectoderm), a fluid-filled cavity (the blastocoel or blastocyst cavity), and a cluster of cells on the interior (the inner cell mass, ICM). The ICM, consisting of undifferentiated cells, gives rise to what will become the fetus if the blastocyst is implanted in a uterus.

Exogenous: Not normally present in a cell, but can be introduced by genetic, biochemical, or other methods. Exogenous nucleic acids include DNA and RNA, which can be single or double-stranded, linear, branched or circular and can be of any length. By contrast, an “endogenous” molecule is one that is normally present in a particular cell at a particular developmental stage under particular environmental conditions.

FokI nuclease: A nonspecific DNA nuclease that occurs naturally in Flavobacterium okeanokoites. The term includes recombinant and mutant forms of the protein, fragments of the FokI nuclease protein, and recombinant and mutant forms thereof that retain nuclease activity that are or may be fused to a DNA-binding polypeptide.

The term “fusion” or “fused” when used in the context of a fusion protein or similar construct means the covalent joining of two polypeptide products (or their corresponding polynucleotides) by genetic engineering. The fused segments may be fused directly to one another but may also be indirectly fused to one another having interceding sequences between the segments of interest.

Heterozygous: A diploid organism is heterozygous at a gene locus when its cells contain two different alleles of a gene. The cell or organism is referred to as a heterozygote specifically for the allele in question; therefore, heterozygosity refers to a specific genotype.

Homology-directed repair (HDR): A mechanism in cells to repair double-stranded DNA lesions. The most common form of HDR is homologous recombination (HR). The HDR repair mechanism can only be used by the cell when there is a homologous piece of DNA present in the nucleus that serves as the repair template and occurs mostly in G2 and S phase of the cell cycle. In contrast, non-homologous end joining (NHEJ) is a pathway that repairs double-strand breaks in DNA, wherein the break ends are directly ligated without the need for a homologous template. NHEJ typically utilizes short homologous DNA sequences called microhomologies as the repair template to guide repair. These microhomologies are often present in single-stranded overhangs on the ends of double-strand breaks. When the overhangs are perfectly compatible, NHEJ usually repairs the break accurately. However, imprecise repair, leading to loss of nucleotides, can also occur and is more common when the overhangs are not compatible.

Isolated: An “isolated” biological component (such as a nucleic acid, peptide, or cell) has been substantially separated, produced apart from, or purified away from other biological components or cells of the organism in which the component naturally occurs (i.e., other chromosomal and extrachromosomal DNA and RNA, cells, and proteins). Nucleic acids, peptides, and proteins that have been “isolated”, thus, include nucleic acids and proteins purified by standard purification methods. The term also embraces nucleic acids, peptides, and proteins prepared by recombinant expression in a host cell as well as chemically synthesized nucleic acids. Cells can also be isolated, such as from other cells or extracellular materials.

Marker or label: An agent capable of detection, for example, by ELISA, spectrophotometry, flow cytometry, immunohistochemistry, immunofluorescence, microscopy, northern analysis, or Southern analysis. For example, a marker can be attached to a nucleic acid molecule or protein, thereby permitting detection of the nucleic acid molecule or protein. Examples of markers include, but are not limited to, radioactive isotopes, nitorimidazoles, enzyme substrates, co-factors, ligands, chemiluminescent agents, fluorophores, haptens, enzymes, and combinations thereof. Methods for labeling and guidance in the choice of markers appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

In some embodiments, the marker is a fluorophore (“fluorescent label”). Fluorophores are chemical compounds, which, when excited by exposure to a particular wavelength of light, emit light (i.e., fluoresce), for example, at a different wavelength. Fluorophores can be described in terms of their emission profile, or “color.” Green fluorophores, for example, Cy3, FITC, and Oregon Green, are characterized by their emission at wavelengths generally in the range of 515-540 λ. Red fluorophores, for example, Texas Red, Cy5, and tetramethylrhodamine, are characterized by their emission at wavelengths generally in the range of 590-690 λ.

In vitro fertilization: The fusion of an oocyte and a sperm in culture outside of body such that a one-cell embryo is formed. In vitro fertilization includes techniques wherein sperm is incubated with eggs in culture to form a one-cell embryo. Intracytoplamic sperm injection (ICSI) is an alternative in vitro fertilization procedure in which a single sperm is injected directly into an egg. The procedure is performed under a microscope using micromanipulation devices. A holding pipette is used to stabilize the mature oocyte with gentle suction applied by a microinjector. From the opposite side, a thin, hollow glass micropipette is used to collect a single sperm, having immobilized it by striking its tail with the point of the micropipette. The micropipette is pierced through the oolema and into the inner part of the oocyte (cytoplasm). The sperm is then released into the oocyte.

Mitotic or Meiotic Spindle: The structure that separates the chromosomes into the daughter cells during cell division. It is part of the cytoskeleton in eukaryotic cells. Depending on the type of cell division, it is also referred to the meiotic spindle during meiosis. The cellular spindle apparatus includes spindle microtubules, associated proteins, and any centrosomes or asters present at the spindle poles. The spindle apparatus is vaguely ellipsoid in shape and tapers at the ends but spreads out in the middle. In the wide middle portion, known as the spindle midzone, antiparallel microtubules are bundled by kinesins. At the pointed ends, known as spindle poles, microtubules are nucleated by the centrosomes in most animal cells.

Meiosis: A process of reductional division in which the number of chromosomes per cell is halved. In animals, meiosis always results in the formation of gametes.

During meiosis, the genome of a diploid germ cell, which is composed of long segments of DNA packaged into chromosomes, undergoes DNA replication followed by two rounds of division, resulting in four haploid cells. Each of these cells contain one complete set of chromosomes, or half of the genetic content of the original cell. Meiosis I separates homologous chromosomes, producing two haploid cells (23 chromosomes, N in humans), so meiosis I is referred to as a reductional division. A regular diploid human cell contains 46 chromosomes and is considered 2N because it contains 23 pairs of homologous chromosomes. However, after meiosis I, although the cell contains 46 chromosomes, it is only considered N because, later, in anaphase I, the sister chromatids will remain together as the spindle pulls the pair toward the pole of the new cell. In meiosis II, an equational division similar to mitosis occurs whereby the sister chromatids are finally split, creating a total of 4 haploid cells (23 chromosomes, N) per daughter cell from the first division.

Thus, meiosis II is the second part of the meiotic process. Much of the process is similar to mitosis. The result is production of four haploid cells (23 chromosomes, 1N in humans) from the two haploid cells (23 chromosomes, 1N*each of the chromosomes consisting of two sister chromatids) produced in meiosis I. The four main steps of meiosis II are: prophase II, metaphase II, anaphase II, and telophase II. In metaphase II, the centromeres contain two kinetochores that attach to spindle fibers from the centrosomes (centrioles) at each pole. The new equatorial metaphase plate is rotated by 90 degrees compared with meiosis I, perpendicular to the previous plate.

Mitosis or Mitotic Cell Division: The type of cell division that results in two daughter cells, each having the same number and kind of chromosomes as the parent nucleus, typical of somatic cell division. Mitosis includes prophase, metaphase, anaphase, and telophase and results in the formation of two new nuclei with the same chromosomal content. The cell cycle consists of four distinct phases: G₁ phase, S phase (synthesis), G₂ phase (collectively known as interphase), and M phase (mitosis). M phase is composed of two tightly coupled processes: karyokinesis, in which the cell's chromosomes are divided, and cytokinesis, in which the cell's cytoplasm divides forming two daughter cells. The S stage of interphase, in which the DNA is replicated, precedes mitosis (i.e., division of the nucleus) and is often accompanied or followed by cytokinesis, in which the cytoplasm, organelles, and cell membrane are divided into two new cells containing roughly equal shares of the cellular components. Combined, mitosis and cytokinesis comprise the mitotic (M) phase of an animal cell cycle (i.e., the division of a mother cell into two daughter cells that are genetically identical).

Mosaic: An individual composed of two different cell types, such as cells heterozygous for a specific allele and other cell homozygous for a particular allele.

Nuclear genetic material: Structures and/or molecules found in the nucleus that comprise polynucleotides (e.g., DNA), which encode information about the individual. Nuclear genetic material includes chromosomes and chromatin. The term also refers to nuclear genetic material (e.g., chromosomes) produced by cell division, such as the division of a parental cell into daughter cells. Nuclear genetic material does not include mitochondrial DNA.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Oocyte: A female gamete or germ cell involved in reproduction, which is also referred to as an egg. A mature egg has a single set of maternal chromosomes (23, X in a human primate) and is halted at metaphase II. A “hybrid” oocyte has the cytoplasm from a first primate oocyte (termed a “recipient”) but does not have the nuclear genetic material of the recipient; it has the nuclear genetic material from another oocyte, termed a “donor.”

Pharmaceutically acceptable carriers: The pharmaceutically acceptable carriers useful in this invention are conventional. Remington's Pharmaceutical Sciences, by E. W. Martin, Mack Publishing Co., Easton, Pa., 15^{th} Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids, such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH-buffering agents, and the like, for example, sodium acetate or sorbitan monolaurate.

Polynucleotide: A nucleic acid sequence (such as a linear sequence) of any length. Therefore, a polynucleotide includes oligonucleotides and gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA and include peptide nucleic acid (PNA) molecules.

Prenatal: Existing or occurring before birth. Similarly, “postnatal” is existing or occurring after birth.

Primate: All animals in the primate order, including monkeys and humans Exemplary non-human primates include, for example, chimpanzees, rhesus macaques, squirrel monkeys, and lemurs. They include Old World, New World, and prosimian monkeys.

Recombination: A process of exchange of genetic information between two polynucleotides. “Homologous recombination (HR)” refers to the specialized form of an exchange that takes place, for example, during repair of double-strand breaks in cells. Nucleotide sequence homology is utilized in recombination, for example, using a “donor” molecule to template repair of a “target” molecule (i.e., the one that experienced the double-strand break). Recombination includes “non-crossover gene conversion” or “short tract gene conversion” because it leads to the transfer of genetic information from the donor to the target.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences. Homologs or variants of a FGF polypeptide will possess a relatively high degree of sequence identity when aligned using standard methods.

Methods of alignment of sequences for comparison are well-known in the art. Various programs and alignment algorithms are described in Smith and Waterman, Adv. Appl. Math., 2:482, 1981; Needleman and Wunsch, J. Mol. Biol., 48:443, 1970; Pearson and Lipman, Proc. Natl. Acad. Sci., USA 85:2444, 1988; Higgins and Sharp, Gene, 73:237, 1988; Higgins and Sharp, CABIOS, 5:151, 1989; Corpet et al., Nucleic Acids Research, 16:10881, 1988; and Pearson and Lipman, Proc. Natl. Acad. Sci. USA, 85:2444, 1988. Altschul, et al., Nature Genet., 6:119, 1994, presents a detailed consideration of sequence alignment methods and homology calculations.

The NCBI Basic Local Alignment Search Tool (BLAST) (Altschul, et al., J. Mol. Biol. 215:403, 1990) is available from several sources, including the National Center for Biotechnology Information (NCBI, Bethesda, Mass.) and on the internet, for use in connection with the sequence analysis programs blastp, blastn, blastx, tblastn, and tblastx. A description of how to determine sequence identity using this program is available on the NCBI website on the internet.

Homologs and variants of a polypeptide are typically characterized by possession of at least about 75%, for example, at least about 80%, sequence identity counted over the full length alignment with the amino acid sequence of the factor using the NCBI Blast 2.0, gapped blastp set to default parameters. For comparisons of amino acid sequences greater than about 30 amino acids, the Blast 2 sequences function is employed using the default BLOSUM62 matrix set to default parameters (gap existence cost of 11 and a per-residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids and may possess sequence identities of at least 85% or at least 90% or 95%, depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Single-stranded nucleic acid: A nucleic acid that only includes a single polymer strand (i.e., the nucleic acid polymer strand does not form non-covalent bonds with another nucleic acid polymer), such as single-stranded DNA (ssDNA). The nucleic acid molecule can be single-stranded in full (e.g., ssDNA formed through melting a double-stranded DNA molecule) or in part (e.g., a ssDNA region formed through damage and/or enzymatic activity).

Site-specific binding: Substantial or preferential binding only to a defined target, such as a nucleic acid, protein, enzyme, polysaccharide, or a small molecule, for example, a nucleotide-binding guide (e.g., a guide RNA, gRNA, of a CRISPR-Cas9 system; a TALE of a TALEN; or a zinc finger of a ZFN) that substantially or preferentially binds only to a defined target nucleic acid sequence within an allele, such as an allele of interest for correction in a primate cell. The determination that a particular agent binds substantially only to a specific polypeptide may readily be made by using or adapting routine procedures (see, e.g., Brazelton et al., GM Crops Food, 6(4): 266-276, 2015).

Subject: Human and non-human animals, including all vertebrates, such as mammals and non-mammals, such as non-human primates, mice, rabbits, sheep, dogs, cats, horses, cows, chickens, amphibians, and reptiles. In many embodiments of the described methods, the subject is a human.

Targeted nuclease: A nuclease directed to a specific site on a nucleic acid. In some examples, the targeted nuclease can be non-naturally occurring (i.e., the targeted nuclease does not exist in nature without artificial aid, such as CRISPR-Cas9, zinc finger nucleases, ZFNs, or transcription activator-like effectors, TALENs).

Totipotent (totipotency): A cell's ability to divide and ultimately produce an entire organism, including all extraembryonic tissues in vivo. In one aspect, the term “totipotent” refers to the ability of the cell to progress through a series of divisions into a blastocyst in vitro. The blastocyst comprises an inner cell mass (ICM) and a trophectoderm. The cells found in the ICM give rise to pluripotent stem cells (PSCs) that possess the ability to proliferate indefinitely or, if properly induced, differentiate in all cell types contributing to an organism. Trophectoderm cells generate extra-embryonic tissues, including placenta and amnion.

As used herein, the term “pluripotent” refers to a cell's potential to differentiate into cells of the three germ layers: endoderm (e.g., interior stomach lining, gastrointestinal tract, and the lungs), mesoderm (e.g., muscle, bone, blood, and urogenital), and ectoderm (e.g., epidermal tissues and the nervous system). Pluripotent stem cells can give rise to any fetal or adult cell type, including germ cells. However, PSCs alone cannot develop into a fetal or adult animal when transplanted in utero because they lack the potential to contribute to all extraembryonic tissue (e.g., placenta in vivo or trophoblast in vitro).

PSCs are the source of multipotent stem cells (MPSCs) through spontaneous differentiation or due to exposure to differentiation induction conditions in vitro. The term “multipotent” refers to a cell's potential to differentiate and give rise to a limited number of related, different cell types. These cells are characterized by their multi-lineage potential and the ability for self-renewal. In vivo, the pool of MPSCs replenishes the population of mature functionally active cells in the body. Among the exemplary MPSC types are hematopoietic, mesenchymal, or neuronal stem cells.

Transplantable cells include MPSCs and more specialized cell types such as committed progenitors as well as cells further along the differentiation and/or maturation pathway that are partly or fully matured or differentiated. “Committed progenitors” give rise to a fully differentiated cell of a specific cell lineage. Exemplary transplantable cells include pancreatic cells, epithelial cells, cardiac cells, endothelial cells, liver cells, endocrine cells, and the like.

Transcription activator-like effector nuclease (TALEN): A DNA-binding protein that includes an array of amino acid repeats (e.g., 33 or 34 amino acid repeats). A TALEN is non-naturally occurring and includes the DNA-cutting domain of a nuclease fused to transcription activator-like effector (TALE) domains. The TALE domain can be engineered to specific DNA sequences. See, for example, Gaj et al., Trends Biotechnol, 31(7): 397-405, 2013, incorporated herein by reference.

Treating, Treatment, and Therapy: Any success or indicia of success in the attenuation or amelioration of an injury, pathology, or condition, including any objective or subjective parameter such as abatement, remission, diminishing of symptoms, or making the condition more tolerable to the patient, slowing in the rate of degeneration or decline, making the final point of degeneration less debilitating, improving a subject's physical or mental well-being, or improving vision. The treatment may be assessed by objective or subjective parameters, including the results of a physical examination, neurological examination, or psychiatric evaluations.

Upstream: A relative position on a polynucleotide, wherein the “upstream” position is closer to the 5′ end of the polynucleotide than the reference point. In the instance of a double-stranded polynucleotide, the orientation of 5′ and 3′ ends are based on the sense strand, as opposed to the antisense strand.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating, or the like.

Wild-type: The phenotype of the typical form of an allele as it occurs in nature. With regard to a gene that affects a disease process, the “wild-type” is the “normal” allele at a locus, in contrast to the allele associated with the disease process, which is the “mutant” allele. Mutant alleles can be the result of insertions, deletions, base pair mutations, and/or frame shift mutations.

Zinc finger DNA-binding domain: A polypeptide domain that binds DNA in a sequence-specific manner through one or more zinc fingers, which are regions of amino acid sequence within the binding domain, whose structure is stabilized through coordination of a zinc ion.

Zinc finger-binding domains, for example, the recognition helix of a zinc finger, can be engineered to bind to a predetermined nucleotide sequence. Rational criteria for design of zinc finger-binding domains include application of substitution rules and computerized algorithms for processing information in a database storing information of existing ZFP designs and binding data, see, for example, U.S. Pat. No. 5,789,538; U.S. Pat. No. 5,925,523; U.S. Pat. No. 6,007,988; U.S. Pat. No. 6,013,453; U.S. Pat. No. 6,140,081; U.S. Patent No.6,200,759; U.S. Pat. No. 6,453,242; U.S. Pat. No. 6,534,261; and PCT Publication Nos. WO 95/19431, WO 96/06166, WO 98/53057, WO 98/53058, WO 98/53059, WO 98/53060, WO 98/54311, WO 00/27878, WO 01/60970, WO 01/88197, WO 02/016536, WO 02/099084, and WO 03/016496, incorporated herein by reference.

Zinc finger nucleases (ZFNs): Non-naturally occurring restriction enzymes generated by fusing a zinc finger DNA-binding domain with a DNA-cleavage domain. See, for example, Gaj et al., Trends Biotechnol, 31(7): 397-405, 2013, incorporated herein by reference.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### Overview of Several Embodiments

Methods are disclosed herein for correcting a mutant allele of a gene of interest in a primate cell. These method include step a), introducing a non-naturally occurring targeted nuclease and site-specific nucleotide-binding guide that work together to introduce double-stranded breaks in the mutant allele into the primate cell, wherein: i) the primate cell is undergoing mitotic cell division; ii) the primate cell includes a genome that is heterozygous for the mutant allele, such that the genome includes one copy of the mutant allele and one copy of a wild-type allele; and iii) single-stranded oligonucleotides homologous to the wild-type allele are not introduced into the primate cell. The targeted nuclease can be a clustered regularly interspaced short palindromic repeats (CRISPR)-associated (Cas)9, zinc finger nuclease (ZFN), or transcription activator-like effector nucelase (TALEN). The method also includes step b), allowing the primate cell to activate homology-directed repair of the double-stranded DNA breaks in the mutant allele, thereby correcting the mutant allele using the normal wild-type allele as a repair template and producing a primate cell that is homozygous for the wild-type allele. In some embodiments, the primate is a human.

In some embodiments, the primate cell is an embryonic cell, such as, but not limited to, a one-cell embryo. The method can include generating an embryo, such as a one-cell embryo, by selecting a primate oocyte comprising a genome having a mutant allele or a wild-type allele of a gene of interest from a primate species, fertilizing the primate oocyte with a sperm from the same primate species, wherein the sperm includes a wild-type allele or a mutant allele of the gene of interest, respectively, thereby forming a one-cell primate embryo, wherein the primate embryo heterozygous and comprises the one copy of the wild-type allele and the one copy of the mutant allele. In specific, non-limiting examples, the targeted nuclease and the site-specific nucleotide-binding guide are introduced into the primate oocyte simultaneously with fertilizing the primate oocyte. In other, non-limiting examples, fertilizing the primate oocyte comprises intracytoplasmic sperm injection (ICSI). In more non-limiting examples, the primate oocyte is at metaphase II when the targeted nuclease and the site-specific nucleotide-binding guide are introduced. In further non-limiting examples, the methods include culturing the embryo to form a multi-cell embryo in vitro. In some examples, the multi-cell embryo is not mosaic for cells comprising the mutant allele.

In additional embodiments, the methods include assaying for successful correction of the mutant allele, such as using Sanger sequencing. In some embodiments, the methods can include comprising assaying for off-target effects, such as using whole-genome sequencing.

In further embodiments, the primate cell is a somatic cell, for example a mesoderm, endoderm, or ectoderm cell. The somatic cell can be a cardiac cell, skin cell, white blood cell, liver cell, pancreatic cell, kidney cell, ovarian cell, testicular cell, prostatic cells breast cell, muscle cell, cell of the digestive system cell of the respiratory system, or an osteogenic cell.

In more embodiments, the primate cell can be a pluripotent or multipotent stem cell. In some specific, non-limiting examples, the primate cell is a bone marrow stem cell, hematopoietic stem cell, mesenchymal stem cell, intestinal stem cell, neuronal stem cell, or dental stem cell.

In some embodiments, the mutant allele comprises a deletion or an insertion as compared to the wild-type allele. In more embodiments, the mutant allele comprises a base pair substitution as compared to the wild-type allele. In further embodiments, the mutant allele comprises a frame shift mutation as compared to the wild-type allele. In some non-limiting examples, the gene of interest is myosin binding protein C (MYBPC3), fibroblast growth factor receptor 3 (FGFR3), serpin family A member 1 (SERPINA1), protein kinase D1 (PKD), breast cancer 1 (BRCA1), breast cancer 2 (BRCA2), glycyl-tRNA synthetase (GARS), WNT signaling pathway regulator (APC), cystic fibrosis transmembrane conductance regulator (CFTR), chimerin 1 (CHN1), dystrophin (DMD), coagulation factor V (F5), fragil X mental retardation 1 (FMR1), glucosylceramidase beta (GBA), homeostatic iron regulator (HFE), coagulation factor IX (FIX), huntingtin (HD), fibrillin 1 (FBN1), dystrophia myotonica protein kinase (DMPK), cellular nucleic acid binding protein (CNBP), protein tyrosine phosphatase, non-receptor type 11 (PTPN11), Ras/Rac guanine nucleotide exchange factor 1 (SOS1), Raf proto-oncogene serine/threonine kinase (RAF1), Kras proto-oncogene GTPase (KRAS), collagen type alpha 1 chain (COL1A1), collagen type alpha 2 chain (COL1A2), synuclein alpha (SNCA), ubiquitin C-terminal hydrolase L1 (UCHL1), leucine rich repeat kinase 2 (LRRK2), Parkinson disease 3 (PARKS), parkin RBR E3 ubiquitin protein ligase (PARK2), parkinsonism associated deglycase (PARK7), PTEN induced putative kinase 1 (PARK6), apolipoprotein B (APOB), low density lipoprotein receptor (LDLR), low density lipoprotein receptor adaptor protein 1 (LDLRAP1), proprotein convertase subtilisin/kexin type 9 (PCSK9), actin alpha cardiac muscle 1 (ACTC1), actinin alpha2 (ACTN2), calreticulin 3 (CALR3), cysteine and glycine rich protein 3 (CSRP3), junctophilin2 (JPH2), myosin heavy chain 7 (MYH7), myosin light chain 2 (MYL2), myosin light chain 3 (MyL3), myozenin 2 (MYOZ2), nexilin F-actin binding protein (NEXN), phospholamban (PLN), protein kinase AMP-activated non-catalytic subunit gamma 2 (PRKAG2), titin-cap (TCAP), troponin I3 cardiac type (TNNI3), troponin T2 cardiac type (TNNT2), tropomyosin 1 (TPM1), titin (TTN), and/or vinculin (VCL).

In other specific, non-limiting examples, (a) the somatic cell is from a human subject that has breast cancer, (b) the somatic cell is a breast cell, and (c) the gene of interest is BRCA1 or BRCA 2. In further non-limiting examples, (a) the somatic cell is from a human subject that has familial cardiomyopathy, (b) the cell is a cardiac cell, and (c) the gene of interest is MYBPC3, ACTC1, ACTN2, CALR3, CSRP3, JPH2, MYH7, MYL2, MyL3, MYOZ2, NEXN, PLN, PRKAG2, TCAP, TNNI3, TNNT2, TPM1, TTN, and/or VCL. In further non-limiting examples, (a) the somatic cell is from a human subject that has familial hypercholesterolemia, (b) the cell is a cardiac cell, and (c) the gene of interest is APOB, LDLR, LDLRAP1, and/or PCSK9).

### CRISPR Cas and Other Targeted Nuclease Systems

Methods and compositions are disclosed herein for altering genes in cells (e.g., primate cells, such as embryos or somatic cells), specifically genes wherein there is one mutant allele and one wild-type allele. The methods and compositions described herein introduce one or more breaks near the site of a gene of interest such that endogenous homologous recombination occurs in in cells (e.g., primate or human cells, such as embryos or somatic cells), such that two wild-type copies of the gene are produced. The primate cell can be a human cell.

In some embodiments, the cell (e.g., a primate or human cell, such as a somatic cell or an embryo) is heterozygous at a gene of interest. Thus, the cell includes one allele with a wild-type sequence and one allele with a mutant allele (to be corrected) with a variant sequence. In some embodiments, the methods disclosed herein can use a targeted nuclease and site-specific nucleotide-binding guide that act together to introduce double-stranded breaks in the mutant allele into the primate cell. The target nuclease can be a CRISPR-Cas (e.g., CRISPR-Cas9) system that introduces double-stranded DNA breaks at the mutant allele, such that the mutant allele is targeted is cleaved by Cas. This results in the introduction of double-stranded breaks. The mutant allele undergoes homology-directed repair based on the wild-type allele, thereby correcting the mutant allele. Thus, a cell homozygous for the wild-type allele is created. In other embodiments, different polynucleotide-binding polypeptides can be used in the methods disclosed herein, provided they induce double-stranded DNA breaks in the mutant allele and not the wild-type allele. In some embodiments, the recombinant polynucleotide-binding polypeptide is a recombinant DNA-binding polypeptide that specifically binds to a genomic target sequence of interest, such as within the mutant allele.

In some embodiments, the site-specific nucleotide-binding guide includes a zinc-finger domain or a transcription activator-like effector (TALE) domain or a polypeptide fragment thereof that retains the DNA-binding function of the TALE domain or the zinc-finger domain. Furthermore, the site-specific nucleotide-binding guide is combined with a targeted nuclease, such as a zinc-finger domain or a transcription activator-like effector (TALE) domain fused to the targeted nuclease, or a fragment thereof. Exemplary nucleases include S1 nuclease, mung bean nuclease, pancreatic DNAase I, micrococcal nuclease, and yeast HO endonuclease; see also Linn et al. (eds.), Nucleases, Cold Spring Harbor Laboratory Press, 1993.

The nuclease can be any nuclease of interest. Restriction endonucleases (restriction enzymes) are present in many species and are capable of sequence-specific binding to DNA (at a recognition site) and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA at nine nucleotides from its recognition site on one strand and 13 nucleotides from its recognition site on the other (see, for example, U.S. Pat. Nos. 5,356,802; 5,436,150 and 5,487,994; Li et al., Proc. Natl. Acad. Sci. USA, 89:4275-4279, 1992; Li et al., Proc. Natl. Acad. Sci. USA, 90:2764-2768, 1993; Kim et al., Proc. Natl. Acad. Sci. USA, 91:883-887, 1994; Kim et al., J. Biol. Chem., 269:31, 978-31, 982, 1994). Thus, in one embodiment, a nuclease domain from at least one Type IIS restriction enzyme is utilized. An exemplary Type IIS restriction enzyme, the cleavage domain of which is separable from the binding domain, is Fok1. This enzyme is active as a dimer. Bitinaite et al., Proc. Natl. Acad. Sci. USA, 95:10,570-10, 575, 1998. Additional forms of Fold nuclease are set forth in U.S. Published Patent Application No. 20110027235, which is incorporated herein by reference. In the case of a recombinant DNA-binding polypeptide produced from a TALE domain, fusion with a polypeptide having nuclease activity forms a transcription activator-like effector nuclease (TALEN). These are of use as the targeted nuclease and site-specific nucleotide-binding guide that act together to introduce double-stranded breaks in the mutant allele.

The targeted nuclease and site-specific nucleotide-binding guide that act together to introduce double-stranded breaks in the mutant allele to target the mutant allele, such as, but not limited to, an allele encoding a non-functional gene product. The mutant allele can have an insertion, deletion, frame shift mutation, or base pair substitution as compared to the wildtype allele. Simple gene disruptions can be generated by cleavage of the target site followed by alteration of nucleic acids, such as a deletion, and repair by homology-directed repair (HDR) in a cell (e.g., a primate cell, such as an embryo or somatic cell) of interest. The cell can be a human cell. As disclosed herein, in specific, non-limiting examples, the targeted nuclease and site-specific nucleotide-binding guide can be introduced into a one-cell embryo or during fertilization of an oocyte, such as during ICSI.

In some embodiments, the targeted nuclease and site-specific nucleotide-binding guide that act together to introduce double-stranded breaks in the mutant allele is a CRISPR system. A typical CRISPR system is composed of two components, a CRISPR-associated nuclease (Cas), such as, but not limited to, Cas9, and one or more guide RNAs (gRNAs), each of which contains (1) a CRISPR RNA (crRNA) and a trans-activating CRISPR RNA (tracrRNA) or (2) a small (or single) guide RNA (sgRNA). Any type of gRNA can be used.

Target recognition by crRNAs occurs through complementary base pairing with target DNA, which directs cleavage of foreign sequences by means of Cas proteins. In some embodiments, DNA recognition by guide RNA and consequent cleavage by the endonuclease requires complementary base-pairing with a protospacer adjacent motif (PAM) (e.g. 5′-NGG-3′) and with a protospacer region in the target. (Jinek, M. et al., Science, 337;816-821, 2012). The PAM motif recognized by a Cas varies for different Cas proteins. One skilled in the art will recognize that any Cas protein can be used in the systems and methods disclosed herein. These include Cas3, Cas8a, Cas5, Cs 8b, Cas8c, Cast10d, Cse1, Cse2, Csy1, Csy2, Csy3, GSU0054, Cas10, Csm2, Cmr5, Cas10, Csx11, Csn2, Cas4, Cpf1, C2c1, C2c3, C2c2, and Cas 9. In one specific, non-limiting example, Cas9 is used.

One Cas9 of use is from Streptococcus pyogenes as depicted in SEQ ID NO: 1 as follows.

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In other embodiments, the Streptococcus pyogenes Cas9 peptide can include one or more of the mutations described in the literature, including, but not limited to, the functional mutations described in Fonfara et al., Nucleic Acids Res., 42(4):2577-90, 2014; Nishimasu H. et al., Cell, 156(5):935-49, 2014; Jinek M et al., Science, 17;337(6096):816-21, 2012; and Jinek M. et al., Science, 343(6176), 2014, all incorporated herein by reference. Thus, in some embodiments, the systems and methods disclosed herein can be used with the wild-type Cas9 protein having double-stranded nuclease activity, Cas9 mutants that act as single-stranded nickases, or other mutants with modified nuclease activity.

A Cas9 includes a catalytically active nuclease domain. In some embodiments, the Cas9 nuclease includes an HNH-like endonuclease and a RuvC-like endonuclease. Thus, in some embodiments, to generate a double-stranded DNA break, the HNH-like endonuclease cleaves the DNA strand complementary to the gRNA, and the RuvC-like domain cleaves the non-complementary DNA strand. A Cas9 endonuclease can be guided to specific genomic targets using specific gRNA (see below).

In some embodiments, the CRISPR/Cas9 system is introduced into cells. In other embodiments, the CRISPR/Cas9 system is produced introduced into the cytoplasm of oocytes ex vivo, such as during fertilization by ICSI.

In other embodiments of the methods disclosed herein, a nucleic acid is introduced encoding the Cas9, and a promoter is operably linked to the nucleic acid encoding Cas9. This promoter provides for cell specific expression of Cas9.

If a nucleic acid encoding Cas9 is utilized, a nucleic acid molecule encoding a marker also can be operably linked to the promoter. Markers include, but are not limited to, enzymes and fluorescent proteins. In one specific non-limiting example, the marker is tdTomato fluorescent protein. In other embodiments, a nucleic acid molecule encoding a marker is not operably linked to the rhodopsin kinase promoter.

As noted above, the Cas9 RNA guide system can include a gRNA, such as a (1) a mature crRNA that is base-paired to trans-activating crRNA (tracrRNA), forming a two-RNA structure, or (2) an sgRNA, either of which direct Cas9 to the locus of a desired double-stranded (ds) break in target DNA, namely at a mutant allele, such as an allele with a mutation at the MYBPC3, BRCA1, and/or BRCA2 gene. In some embodiments, the tracrRNA and crRNA gRNA is used, and the base-paired tracrRNA:crRNA combination is engineered as a single RNA chimera to produce a guide sequence (e.g., gRNA) that preserves the ability to direct sequence-specific Cas9 dsDNA cleavage (see Jinek, M., et al., Science, 337:816-821, 2012). In some embodiments, the Cas9-guide sequence complex results in cleavage of one or both strands at a target sequence within the mutant allele, such as but not limited to, an allele with a mutation at the MYBPC3, BRCA1, and/or BRCA2 gene. Thus, the Cas9 endonuclease (Jinek, M., et. al., Science, 2012; Mali, P. et al., Nat Methods, 10(10): 1028-1034, 2013) and the gRNA molecules are used for sequence-specific target recognition, cleavage, and genome editing using endogenous repair mechanisms of a mutant allele, such as an allele with a mutation the MYBPC3, BRCA1, and/or BRCA2 gene. The cleavage can be double-stranded cleavage.

In some embodiments, the gRNA molecule is selected so that the target genomic targets bear a protospacer adjacent motif (PAM). In some embodiments, DNA recognition by guide RNA and consequent cleavage by the endonuclease requires the presence of a protospacer adjacent motif (PAM) (e.g. 5′-NGG-3′) immediately after the target.

In some embodiments, cleavage occurs at a site approximately 3 base pairs upstream from the PAM. In some embodiments, the Cas9 nuclease cleaves a double-stranded nucleic acid sequence.

In some embodiments, the guide sequence is selected to reduce the degree of secondary structure within the sequence. Secondary structure may be determined by any suitable polynucleotide folding algorithm. Some programs are based on calculating the minimal Gibbs free energy. An example of one such algorithm is mFold (Zuker and Stiegler, Nucleic Acids Res., 9, 133-148, 1981). Another example folding algorithm is the online webserver RNAfold, which uses the centroid structure prediction algorithm (see, e.g., A.R. Gruber et al., Cell, 106(1): 23-24, 2008; and PA Can and GM Church, Nature Biotechnology, 27(12): 1151-62, 2009). Guide sequences can be designed using the MIT CRISPR design tool found at crispr.mit.edu or the E-CRISP tool found at on the internet at e-crisp.org. Additional tools for designing guide sequences are described in Naito Y et al., Bioinformatics, 2014, and Ma et al., BioMed Research International, Volume 2013, Article ID 270805, 2013. The crRNA or the DNA recognition sequence (“target sequence”) in an sgRNA can be 18-48 nucleotides in length. The crRNA or target sequence can be at least 18, 19, 20, 21, 22, 23, 24, 25, 26, 27, 28, 29, 30, 31, 32, 33, 34, 35, 68, 37, 38, 39, 40, 41, 42, 43, 44, 45, 46, 47, or 48 nucleotides long or about 18-23, 23-28, 28-33, 33-38, 38-43, or 43-48 nucleotides long or about 19 or 20 nucleotides long. In some examples, the crRNA or target sequence is 19 or 20 nucleotides long. In specific examples, sgRNA is used. Exemplary target sequences for an sgRNA targeting MYBCP are as follows:

(SEQ ID NO: 2)
GGTGGAGTTTGTGAAGTAT
(SEQ ID NO: 3)
GGGTGGAGTTTGTGAAGTAT.

Exemplary sgRNA sequences targeting MYBCP are as follows:

(SEQ ID NO: 4)
GGTGGAGTTTGTGAAGTATGUUUUAGAGCUAGAAAUAGCUUAAAAUAAGG
CUAGUCCGUUAUCAACUUGAAAAAGUGGCACCGAUCGGUGCUUUUUU
(SEQ ID NO: 5)
GGGTGGAGTTTGTGAAGTATGUUUUAGAGCUAGAAAUAGCUUAAAAUAAG
GCUAGUCCGUUAUCAACUUGAAAAAGUGGCACCGAUCGGUGCUUUUUU.

Other gRNA sequences are known in the art. For example, BRCA gRNA sequences are available on the GENSCRIPT website (genscript.com, incorporated by reference as available on Apr. 15, 2018). A variety of gRNA sequences are available in databases; see, for example, genscript.com, as available on Apr. 15, 2018.

### Methods for Correcting a Mutant Allele of Interest in a Primate Cell

A method for correcting a mutant allele of a gene of interest in a primate cell. These method include step a), introducing a non-naturally occurring targeted nuclease and site-specific nucleotide-binding guide that work together to introduce double-stranded breaks in the mutant allele into the primate cell, wherein: i) the primate cell is undergoing mitotic cell division; ii) the primate cell includes a genome that is heterozygous for the mutant allele, such that the genome includes one copy of the mutant allele and one copy of a wild-type allele; and iii) single-stranded oligonucleotides homologous to the wild-type allele are not introduced into the primate cell. In some examples, the targeted nuclease can be clustered regularly interspaced short palindromic repeats (CRISPR)-associated (Cas)9, zinc finger protein (ZNF), or transcription activator-like effectors (TALEN). The method also includes step b), allowing the primate cell to activate homology-directed repair of the double-stranded DNA breaks in the mutant allele, thereby correcting the mutant allele using the normal wild-type allele as a repair template and producing a primate cell that is homozygous for the wild-type allele. In some embodiments, the primate is a human. Suitable cells are disclosed below.

In some embodiments of the disclosed methods, single-stranded nucleic acids, such as a repair template is not utilized. A repair template is a single-stranded DNA, that can include about 40 to about 90 base pairs homologous to the region where the DNA break occurs, such as about at least 40, at least 50, at least 60, at least 70, at least 80, or at least 90 or about 40-50, 50-60, 60-70, 70-80, or 80-90 base pairs. In these embodiments, a repair template homologous to the wild-type allele is not introduced into the cell.

Any mutant allele can be corrected using the methods disclosed herein. In some embodiments, the mutant allele includes a deletion and/or an insertion as compared to the wild-type allele. In other embodiments, the mutant allele includes a base pair substitution as compared to the wild-type allele. In further embodiments, the mutant allele includes frame shift mutation as compared to the wild-type allele. It is understood that any allele can be corrected using the methods disclosed herein, provided the embryo is heterozygous for the allele.

Methods for correcting a mutant allele of a gene of interest in a primate cell (e.g., a human cell and/or an embryo) are disclosed herein. More than one allele can be corrected, such as at least 1, 2, 3, 4, 5, 10, 15, 20, or 25 or 1-2, 2-3, 3-4, 4-5, 5-10, 10-, 15, 15-20, or 20-25 alleles.

In some embodiments, the disclosed methods utilize a CRISPR/Cas9 System. More than one DNA break can be introduced in more than one allele by using more than one gRNA. For example, two gRNAs can be utilized, such that two breaks are achieved in two different alleles, both of which are present in the cell in heterozygous form. When two or more gRNAs are used to position two or more cleavage events in a target nucleic acid, it is contemplated that, in an embodiment, the two or more cleavage events may be made by the same or different Cas9 proteins. For example, when two gRNAs are used to position two double-strand breaks, a single Cas9 nuclease may be used to create both double-strand breaks.

The methods can include introducing a non-naturally occurring targeted nuclease and site-specific nucleotide-binding guide that act together to introduce double-stranded breaks in the mutant allele into the primate cell. In some examples, the primate cell is undergoing mitotic cell division. The primate cell includes a genome that is heterozygous for the mutant allele, for example, such that the genome comprises one copy of the mutant allele and one copy of a wild-type allele. In the methods herein, single-stranded oligonucleotides homologous to the wild-type allele are not necessary, and, thus, the methods can be performed where single-stranded oligonucleotides homologous to the wild-type allele are not introduced into the primate cell. The methods herein can allow the primate cell to activate homology-directed repair of the double-stranded DNA breaks in the mutant allele. Thereby, the methods can correct a mutant allele using the normal wild-type allele as a repair template and producing a primate cell that is homozygous for the wild-type allele.

Any non-naturally occurring targeted nuclease and site-specific nucleotide-binding guide that act together to introduce double-stranded breaks in the mutant allele into the primate cell can be used. The nuclease and site-specific nucleotide-binding guide can be separate molecules or can be fused together. In some examples, the nuclease and guide are separate molecules that act together to introduce double-stranded breaks in the mutant allele, such as a clustered regularly interspaced short palindromic repeats (CRISPR)-associated (Cas) system (e.g., CRISPR-Cas9). For example, the CRISPR-Cas9 system can include a guide nucleic acid (e.g., a guide RNA, “gRNA”) specific for a gene of interest. In other examples, the nuclease and site-specific nucleotide-binding guide can be fused together, such as a zinc finger nuclease (ZFN) or transcription activator-like effector nuclease (TALEN). For example, the ZFN or TALEN can include a zinc finger domain or transcription activator-like effector (TALE) domain specific for the gene of interest.

In embodiments, the methods can further include assaying for successful correction of the mutant allele. Successful correction of the allele can include assaying for one or more aspects of successful correction, such as correction of the mutant allele and avoiding off-target effects. In some examples, successful correction can include assaying for correction of the mutant allele (i.e., on-target validation). On-target validation assays are known in the art. Any on-target validation assay can be used. Exemplary assays for correction of the mutant allele include Sanger sequencing, mismatch sequencing, and targeted deep sequencing (see, e.g., Brinkman et al., Nucleic Acids Res., 42(22): e168, 2014, and Tycko et al., Mol Cell, 63(3): 355-370, 2016, both of which are incorporated herein by reference). In some examples, successful correction of the mutant allele includes assaying for off-target effects, such as non-specific and/or unintended point mutations, deletions, insertions, inversions, and translocations, either predictable or unpredictable. Off-target validation assays are known in the art. Any off-target validation assay can be used. Exemplary off-target validation assays include biased methods (i.e., methods directed to predicatable off-target effect), such as Sanger sequencing, mismatch sequencing, and targeted deep sequencing (see, e.g., Brinkman et al., Nucleic Acids Res., 42(22): e168, 2014, and Tycko et al., Mol Cell, 63(3): 355-370, 2016, both of which are incorporated herein by reference), and unbiased methods (i.e., methods directed to unpredictable off-target effects), such as whole-exon sequencing (WES; e.g., using whole genome sequencing and/or microarray genotyping), whole-genome sequencing (WGS), direct in situ breaks labeling, enrichment on streptavidin, and next-generation sequencing (BLESS), high-throughput, genome-wide translocation sequencing (HTGTS), genome-wide, unbiased identification of double-strand breaks (DSBs) enabled by sequencing (GUIDE-seq), integration-deficient lentiviral vector (IDLY) capture, Digenome-seq, and circularization for in vitro reporting of cleavage effects by sequencing (CIRCLE-Seq; see, e.g., Tycko et al., Mol Cell, 63(3): 355-370, 2016; Zhang et al., Mol Ther Nucleic Acids, 4:e264, 2015; Tsai et al., Nat Methods, 14(6):607-614, 2017, detailing unbiased off-target validation assays, all of which are incorporated herein by reference).

The methods can be used to correct any mutant allele in any primate cell capable of mitosis (all GENBANK® accession nos. listed herein are incorporated by reference, as available on Apr. 20, 2017). In some examples, the methods include correcting a mutant allele in a primate cell that causes or plays a role in causing a disorder and/or disease in the primate (i.e., genetic disorders and/or genetic diseases). Genetic disorders and genetic diseases as well as related mutant alleles are known in the art, see the internet at rarediseases.info.nih.gov, as available on Apr. 15, 2018, incorporated herein by reference. Any of these mutant alleles can be corrected using the disclosed methods. In some examples, the genetic disorder and/or genetic disease leads to and/or plays a role in cardiomyopathy, such as familial cardiomyopathy. Exemplary genes wherein a mutant allele can lead to and/or play a role in cardiomyopathy (e.g., familial cardiomyopathy) include myosin binding protein C (MYBPC3, for example, GENBANK® accession no. NG_007667.1), actin alpha cardiac muscle 1 (ACTC1, for example, GENBANK® accession no. NG_007553.1), actinin alpha2 (ACTN2, for example, GENBANK® accession no. NG_009081.1), calreticulin 3 (CALR3, for example, GENBANK® accession no. NG_031959.2), cysteine and glycine rich protein 3 (CSRP3, for example, GENBANK® accession no. NG_011932.2), junctophilin2 (JPH2, for example, GENBANK® accession no. NG_031867.1), myosin heavy chain 7 (MYH7, for example, GENBANK® accession no. NG_007884.1), myosin light chain 2 (MYL2, for example, GENBANK® accession no. NG_007554.1), myosin light chain 3 (MyL3, for example, GENBANK® accession no. NG_007555.2), myozenin 2 (MYOZ2, for example, GENBANK® accession no. NG_029747.1), nexilin F-actin binding protein (NEXN, for example, GENBANK® accession no. NG_016625.1), phospholamban (PLN, for example, GENBANK® accession no. NG_009082.1), protein kinase AMP-activated non-catalytic subunit gamma 2 (PRKAG2, for example, GENBANK® accession no. NG_007486.1), titin-cap (TCAP, for example, GENBANK® accession no. NG_008892.1), troponin 13 cardiac type (TNNI3, for example, GENBANK® accession no. NG_007866.2), troponin T2 cardiac type (TNNT2, for example, GENBANK® accession no. NG_007556.1), tropomyosin 1 (TPM1, for example, GENBANK® accession no. NG_007557.1), titin (TTN, for example, GENBANK® accession no. NG_011618.3), and vinculin (VCL, for example, GENBANK® accession no. NG_008868.1). All GENBANK® accession nos. are incorporated by references as available on Apr. 20, 2017.

In the studies disclosed below, the MYBPC3 gene is used as the target. However, it is understood that any allele can be corrected using the methods disclosed herein, provided the embryo is heterozygous for the allele. Other mutant genes that cause cardiomyopathy include MYH7, TNNT2, and TNNI3. Thus, in some non-limiting examples, a mutant allele of MYH7, TNNT2, and/or TNNI3 is corrected using the disclosed methods.

In some examples, the genetic disorder and/or genetic disease leads to and/or plays a role in hypercholesterolemia, such as familial hypercholesterolemia. Exemplary genes wherein a mutant allele can lead to hypercholesterolemia (e g , familial hypercholesterolemia) include apolipoprotein B (APOB, for example, GENBANK® accession no. NG_011793.1), low density lipoprotein receptor (LDLR, for example, GENBANK® accession no. NG_009060.1), low density lipoprotein receptor adaptor protein 1 (LDLRAP1, for example, GENBANK® accession no. NG_008932.1), and proprotein convertase subtilisin/kexin type 9 (PCSK9, for example, GENBANK® accession no. NG_009061.1).

In some examples, the mutant allele can lead to and/or play a role in other genetic disorders and/or genetic diseases, such as mutant alleles at the following genes: fibroblast growth factor receptor 3 (FGFR3, for example, GENBANK® accession no. NG_012632.1), serpin family A member 1 (SERPINA1, for example, GENBANK® accession no. NG_008290.1), protein kinase D1 (PKD, for example, GENBANK® accession no. NG_052879.1), breast cancer 1 (BRCA1, for example, GENBANK® accession no. NG_005905.2), breast cancer 2 (BRCA2, for example, GENBANK® accession no. NG_012772.3), glycyl-tRNA synthetase (GARS, for example, GENBANK® accession no. NG_007942.1), WNT signaling pathway regulator (APC, for example,

GENBANK® accession no. NG_008481.4), cystic fibrosis transmembrane conductance regulator (CFTR, for example, GENBANK® accession no. NG_016465.4), chimerin 1 (CHN1, for example, GENBANK® accession no. NG_012642.1), dystrophin (DMD, for example, GENBANK® accession no. NG_012232.1), coagulation factor V (F5, for example, GENBANK® accession no. NG_011806.1), fragil X mental retardation 1 (FMR1, for example, GENBANK® accession no. NG_007529.2), glucosylceramidase beta (GBA, for example, GENBANK® accession no. NG_009783.1), homeostatic iron regulator (HFE, for example, GENBANK® accession no. NG_008720.2), coagulation factor IX (FIX, for example, GENBANK® accession no. NG_007994.1), huntingtin (HD, for example, GENBANK® accession no. NG_009378.1), fibrillin 1 (FBN1, for example, GENBANK® accession no. NG_008805.2), dystrophia myotonica protein kinase (DMPK, for example, GENBANK® accession no. NG_009784.1), cellular nucleic acid binding protein (CNBP, for example, GENBANK® accession no. NG_011902.1), protein tyrosine phosphatase, non-receptor type 11 (PTPN11, for example, GENBANK® accession no. NG_007459.1), Ras/Rac guanine nucleotide exchange factor 1 (SOS1, for example, GENBANK® accession no. NG_007530.1), Raf proto-oncogene serine/threonine kinase (RAF1, for example, GENBANK® accession no. NG_007467.1), Kras proto-oncogene GTPase (KRAS, for example, GENBANK® accession no. NG_007524.1), collagen type alpha 1 chain (COL1A1, for example, GENBANK® accession no. NG_007400.1), collagen type alpha 2 chain (COL1A2, for example, GENBANK® accession no. NG_007405.1), synuclein alpha (SNCA, for example, GENBANK® accession no. NG_011851.1), ubiquitin C-terminal hydrolase L1 (UCHL1, for example, GENBANK® accession no. NG_012931.1), leucine rich repeat kinase 2 (LRRK2, for example, GENBANK® accession no. NG_011709.1), Parkinson disease 3 (PARK3, for example, Gene ID 5072, location 2p13, as available on Apr. 20, 2017, incorporated herein by reference), parkin RBR E3 ubiquitin protein ligase (PARK2, for example, GENBANK® accession no. NG_008289.2), parkinsonism associated deglycase (PARK7, for example, GENBANK® accession no. NG_008271.1), or PTEN induced putative kinase 1 (PARK6, for example, GENBANK® accession no. NG_008164.1). All GENBANK® accession nos. are incorporated by references as available on Apr. 20, 2017. In some embodiments, the mutant allele is involved in inherited breast cancer. In specific non-limiting examples, a BRCA1 or BRCA2 gene is used as the target.

In some examples, the primate cell is an zygote, oocyte, or stem cell, and the mutant allele is at least one of MYBPC3, FGFR3, SERPINA1, PKD, BRCA1, BRCA2, GARS, APC, CFTR, CHN1, DMD, F5, FMR1, GBA, HFE, FIX, HD, FBN1, DMPK, CNBP, PTPN11, SOS1, RAF1, KRAS, COL1A1, COL1A2, SNCA, UCHL1, LRRK2, PARK3, PARK2, PARK7, PARK6, LDLR, LDLRAP1, ACTC1, ACTN2, CALR3, CSRP3, JPH2, MYH7, MYL2, MyL3, MYOZ2, NEXN, PLN, PRKAG2, TCAP, TNNI3, TNNT2, TPM1, TTN, VCL, APOB, LDLR, LDLRAP1, and/or PCSK9. In some examples, the primate cell is a cardiac stem cell or cardiac somatic cell, and the mutant allele is at least one of MYBPC3, ACTC1, ACTN2, CALR3, CSRP3, JPH2, MYH7, MYL2, MyL3, MYOZ2, NEXN, PLN, PRKAG2, TCAP, TNNI3, TNNT2, TPM1, TTN, VCL, APOB, LDLR, LDLRAP1, and/or PCSK9. In specific examples, the methods include selecting a subject with cardiomyopathy, where the primate cell is a human somatic cardiac cell, and the mutant allele is at least one of MYBPC3, ACTC1, ACTN2, CALR3, CSRP3, JPH2, MYH7, MYL2, MyL3, MYOZ2, NEXN, PLN, PRKAG2, TCAP, TNNI3, TNNT2, TPM1, TTN, and/or VCL. In specific examples, the methods include selecting a subject with hypercholesterolemia, where the primate cell is a human somatic cell, and the mutant allele is at least one of APOB, LDLR, LDLRAP1, and/or PCSK9.

The methods described herein are generally applicable to cells, such as primate cells (both non-human primate and human cells). The cell can be a somatic cell. The cell can be the cell of an embryo, including but not limited to, a one-cell embryo.

Generally, the methods herein can be applied to any cell that is mitotic. Exemplary cells include somatic cells, such as mesoderm, endoderm, and ectoderm cells. Exemplary cells are also the cells from any tissue or organ, including, but not limited to, cardiac, skin, white blood, liver, pancreatic, kidney, ovarian, testicular, prostatic, breast, muscle, and osteogenic cells. Cells of use include cells of the digestive or respiratory system. The cells can be stem cells, such as pluripotent or multipotent stem cells. Exemplary stem cells include bone marrow, hematopoietic, mesenchymal, intestinal, neuronal, and dental stem cells.

In some embodiments, the cell is a one-cell embryo. The embryo can be from a wide array of mammalian animals, including veterinary mammals, such as, but not limited to, livestock mammals, wild animals, domestic mammals, model animal mammals, zoo mammals, and human or non-human primates. As used herein “livestock mammals” refer to any mammalian animal that is useful in an agricultural or livestock setting, such as a pig (porcine), cattle (bovine), sheep (ovine), goat, horse, or buffalo. “Domestic mammals” refer herein to any mammal that has been domesticated by humans such that they are tame and depend upon man for survival, such as a cat (feline), dog (canine), rabbit, guinea pig, and hamster. “Wild” mammals refer to mammals found in the wild setting, such as wild cats. “Model animal mammals” refer herein to any mammal used for scientific and health related research, such as mice, rabbits, and rats. In certain embodiments, these categories of mammals may overlap; for instance, domestic mammals such as dogs may also be classified as a model animal mammal. The disclosed methods are effective in any mammalian species.

The mammal can be a primate, such as a human, or can be a non-human primate. In embodiments, the primate cell can be an embryo (e.g., a one-cell embryo). The primate cell can be a somatic cell.

In some examples, the methods can include generating an embryo prior to introducing a non-naturally occurring targeted nuclease and site-specific nucleotide-binding guide nucleic acid molecule. For example, generating an embryo can include selecting a primate oocyte that includes a genome with a mutant allele or a wild-type allele of a gene of interest from a primate. Generating any embryo can also include fertilizing the primate oocyte with a sperm from the same primate species. In some examples, the sperm can include a wild-type allele or a mutant allele of the gene of interest. Thereby, the methods can be used to form a one-cell primate embryo that is heterozygous (e.g., including one copy of the wild-type allele and one copy of the mutant allele). In some examples, the targeted nuclease and the guide are introduced into the primate oocyte simultaneously with fertilizing the primate oocyte. Any method of fertilization can be used, including intracytoplasmic sperm injection (ICSI) or in vitro fertilization (IVF). In specific examples, ICSI is used for fertilization. The targeted nuclease and site-specific nucleotide-binding guide can be introduced to any cell at any stage that is capable of mitosis, such as a primate oocyte, for example, at metaphase II.

In some embodiments, methods are disclosed wherein a targeted nuclease and site-specific nucleotide-binding guide, such as in a CRISPR/Cas system (or in another system that introduces double-stranded DNA breaks, such as a TALEN or ZFN), are introduced into an oocyte. Primate oocytes, such as human oocytes, can be obtained by using protocols that stimulate a female (e.g., primates, such as humans) to produce a number of viable oocytes. Examples of such stimulation protocols are disclosed in the Examples Section below and in Zelinski-Wooten, et al., Hum. Reprod., 10:1658-1666, 1995. The method of harvesting can also be important in obtaining high-quality oocytes. In one example, the primate oocytes can be harvested using methods known in the art, such as follicular aspiration, and then separated from contaminating blood cells. As an alternative, primate oocytes can be generated from pluripotent stem cells in vitro.

In one aspect, when primates are stimulated to produce oocytes (such as hormonally) and these oocytes are harvested, the oocytes that are collected can be in different phases. Some oocytes are in metaphase I, while other oocytes are in metaphase II. In such cases, the oocytes that are in metaphase I can be put into culture until they reach metaphase II and then used in the methods disclosed herein. Oocytes can be frozen for further use. Thus, in some embodiments, the oocyte was cryopreserved.

An oocyte can be fertilized in vitro. Protocols for performing in vitro fertilization (IVF) can be found at, for example, U.S. Pat. Nos. 4,589,402 and 4,725,579 and in The Handbook of in vitro Fertilization, eds. Trouson and Gardner, Informa Health Care Publ., 2000, as well as In vitro Fertilization and Embryo Culture: A Manual of Basic Techniques, ed. Wolf, Springer Publ., 1988, all incorporated herein by reference in their entireties. There are several issues associated with success in performing IVF. Those issues include, but are not limited to, zona pellucida hardening that leads to decrease in sperm penetration, temperature of fertilization and maintenance of eggs, sperm and embryos, pH, the occurrence of volatile organic compounds found in laboratory air that can harm the process, and other environmental factors. Generally, fertilization uses an oocyte and sperm from the same species.

An exemplary protocol for fertilization includes incubation of hybrid oocytes with the sperm in culture media for about 4-12 hours, such as about at least 4, at least 5, at least 6, at least 7, at least 8, at least 9, at least 10, at least 11, or at least 12 hours or about 5-6, 6-7, 7-8, 8-9, 9-10, 10-11, or 11-12 hours or about 5-11 hours, such as about 8 hours. Fertilization is complete with the observation of two pronuclei in the embryo. However, if conventional WF is not realized, for example, due to consequences of oocyte manipulation, a single sperm can be directly injected into the oocyte using intracytoplasmic sperm injections (ICSI). ICSI involves injection of the sperm into the hybrid oocyte, ordinarily through a glass pipette. The methods disclosed herein can include placing sperm in an ICSI medium, capturing the sperm by drawing the medium containing sperm into the pipette, inserting the pipette containing medium and sperm into the hybrid oocyte, and, following insertion into the hybrid oocyte, transferring the medium containing sperm from the pipette into the hybrid oocyte. ICSI methods for use in primates are disclosed in U.S. Patent Publication No. 20030221206, which also discloses “transICSI” methods that result in the production of embryos, including heterologous DNA.

The targeted nuclease and site-specific nucleotide-binding guide, such as CRISPR/Cas9 or any other system that introduces specific double-stranded DNA breaks, such as a TALEN or ZFN, can be introduced into an oocyte by including them with the sperm during the ICSI fertilization procedure. Thus, the introduction is simultaneous with ferritization so that the targeted nuclease and site-specific nucleotide-binding guide are introduced into a one-cell embryo. Alternatively, the CRISPR/Cas9, TALEN, or ZFN can be introduced into a one-cell zygote, as disclosed above, after fertilization as a separate procedure. The targeted nuclease and site-specific nucleotide-binding guide can be introduced, for example, about at least 5, at least 10, at least 15, at least 20, at least 25, at least 30, at least 35, at least 40, at least 45, at least 50, at least 55, or at least 60 minutes following fertilization. The targeted nuclease and site-specific nucleotide-binding guide can be introduced within about at least 1, at least 2, at least 3, at least 4, or at least 5 hours after fertilization.

If ICSI is utilized, the ICSI medium generally includes the constituents water, ionic constituents, and a buffer. In some embodiments, the medium lacks phosphate. The buffer used in medium can be MOPS or HEPES. Additionally, the ICSI medium may be supplemented with the carbohydrates lactate and pyruvate, and the medium may be further supplemented with one or more of the nonessential acids most abundant in the oocyte: glutamine, glycine, proline, serine, and taurine. In one formulation, the ICSI medium used is supplemented with hyaluronate or polyvinylpyrolidone (PVP) to slow or immobilize the sperm so that they may be captured by pipette for the ICSI process. The targeted nuclease and site-specific nucleotide-binding guide, such as the CRISPR/Cas9, TALEN, or ZFN can be included in this ICSI medium.

When ICSI is performed, a one-cell embryo is formed. This one-cell embryo is totipotent and (i) is capable of four or more cell divisions; (ii) maintains a normal karyotype while in culture; and (iii) is capable of producing a pregnancy and healthy offspring.

The one-cell embryo can be cultured in vitro such that it divides. In some embodiments, the efficiency of producing an 8-cell embryo is greater than about 5%, such as greater than about 10%, greater than about 20%, greater than about 30%, greater than about 40%, greater than about 50%, greater than about 60%, greater than about 70%, greater than about 80% greater than about 90%, or greater than about 95%. In this context, “about” indicates within 1%.

In embodiments, the methods can further include culturing an embryo to form a multi-cell embryo in vitro. A one-cell embryo can be cultured in vitro, wherein the one-cell embryo divides, thereby producing a two-cell, four-cell, or eight-cell embryo; a morula; or a blastocyst. In some examples, the multi-cell embryo is not mosaic for cells comprising the mutant allele. Methods for culturing embryos are well-known in the art, see, for example, U.S. Published Patent Application No. 2009/0004740, which is incorporated herein by reference. In some embodiments, the embryo is not mosaic for cells heterozygous for the mutant allele.

### Use of Cells Produced by the Disclosed Methods

Embryos and cells produced using the methods disclosed herein have a variety of uses. When a one-cell embryo is used in the disclosed methods, a pregnancy can be established. For example, the one-, two-, four-, or eight-cell embryo; morula; or blastocyst can be introduced into the recipient from which the recipient oocyte was isolated. In one example, the recipient is a primate. In another example, the one-, two-, four-, or eight-cell embryo; morula; or blastocyst can be introduced into a surrogate recipient, such as a primate, of the same species, wherein the surrogate animal is different from the first and/or the second primate. Generally, the pregnancy is established in an animal of the same species as the oocyte donor.

The embryo can be allowed to develop to term. Methods for the introduction of embryos into a female and use of surrogate females to produce offspring are well-known in the art. In one example, the donor oocyte, recipient oocyte, and surrogate primate are human. However, in other examples, the donor oocyte, recipient oocyte, and surrogate primate are non-human primates, such as rhesus monkeys or macaques. In some embodiments, the resultant offspring is not mosaic for cells heterozygous for the mutant allele.

Embryos can also be used for production of stem cells. Following fertilization, the resultant embryo is not transplanted into a recipient, but is cultured in vitro. In some embodiments, an embryonic cell is removed from the embryo, and the methods disclosed herein are performed on this cell. The embryo need not be destroyed, as it is viable, and can be implanted into a female or cryopreserved. The single cell of the embryo that has been removed can be treated using the disclosed methods.

Following use of the presently claimed method, the embryo (or embryonic cell) can be cultured and used to produce homozygous cells, such as stem cells. Methods of culturing primate embryos and stem cells are well-known in the art. Any cell culture media that can support the growth and differentiation of human or non-human primate embryonic stem cells can be used. In some embodiments, the pluripotent stem cells are cultured on a feeder layer, such as a layer of murine or primate embryonic fibroblasts. However, the feeder layer can be any cells that support the growth of embryonic stem cells (ESCs). This approach makes for a completely autologous culturing system, thereby eliminating the risk of cross-species contamination. For therapeutic use, the culturing methods can be xeno-free (no xenogeneic cells or components) and, additionally, avoid the use of serum (such as fetal bovine serum, FBS) in the culturing media.

In some embodiments, homozygous non-human or human primate totipotent stem cells (TSCs) or pluripotent stem cells (PSCs) are made using the methods disclosed herein. These stem cells have a variety of uses. TSCs or PSCs can readily be produced from human and non-human primate embryos. In one embodiment, primate TSCs or PSCs are isolated and subsequently cultured in “ES medium,” which supports the growth of embryonic stem cells. The PSCs express SSEA-3, SSEA-4, TRA-1-60, and TRA-1-81. For example, ES medium comprises 80% Dulbecco's modified Eagle's medium (DMEM; no pyruvate, high glucose formulation, Gibco BRL), with 20% fetal bovine serum (FBS; Hyclone), 0.1 mM β-mercaptoethanol (Sigma), and 1% non-essential amino acid stock (Gibco BRL).

In one example, a primate oocyte from a recipient primate is enucleated, and nuclear material, including chromosomes from a donor primate oocyte, is inserted into the enucleated oocyte, as described herein. The resultant hybrid oocyte is then fertilized using sperm from a male of the same species, and a one-cell embryo is formed and treated using the disclosed methods.

Following treatment with the disclosed methods, resultant homozygous TSCs can be cultured in medium, such as but not limited to protein-free HECM-9 medium and cultured at 37° C. in about 5-6% CO₂ until use. These cultures can be maintained under paraffin oil. Once the TSCs reach about the 2-cell stage or beyond, such as the 4-, 8-, or 16-cell stage, the cells can be transferred for further culture or transplantation. In one embodiment, these TSCs are cultured to the blastocyst stage in a culture medium, such as, but not limited to, HECM-9 medium.

In some embodiments, the zonae pellucidae of selected expanded blastocysts are be removed by brief exposure (45-60 seconds) to 0.5% pronase in TH3 medium. In some embodiments, an inner cell mass (ICM) can be isolated from trophectoderm cells by immunosurgery, where zona-free blastocysts are exposed to rabbit anti-rhesus spleen serum for about 30 minutes at about 37° C. After extensive washing (such as using TH3 medium), embryos are incubated in guinea pig complement reconstituted with HECM-9 (1:2, v/v) for about an additional 30 minutes at about 37° C. Partially lysed trophectodermal cells are mechanically dispersed by gentle pipetting, such as with a small bore pipette (for example, about a 125 μn in inner diameter; Stripper pipette, Midatlantic Diagnostics Inc., Marlton, N.J.) followed by the rinsing of ICMs three times, such as with TH3 medium. Isolated ICMs are plated onto a solid substrate, such as onto Nunc 4-well dishes containing mitotically-inactivated feeder layers consisting of mouse embryonic fibroblasts (mEFs); cultured, such as in DMEM/F12 medium (Invitrogen) with glucose and without sodium pyruvate supplemented with 1% nonessential amino acids (Invitrogen), 2 mM L-glutamine (Invitrogen), 0.1 mM β-mercaptoethanol, and 15% FBS; and maintained under conditions at about 37° C. with about 3% CO2, about 5% O₂, and about 92% N₂ gas. Alternatively, whole, intact blastocysts can be directly plated onto mEFs for ESC isolation. Alternatively, trophectoderm can be removed mechanically, for example, using laser-assisted dissection or microscalpel.

After about 1 to about 7 days, cells, such as blastocysts or ICMs that are attached to the feeder layer and with initiated outgrowth, can be dissociated into small cell clumps, such as manual dissociation with a microscalpel, and re-plated onto a new substrate, such as new embryonic fibroblasts (mEFs). After the first passage, colonies with embryonic stem cell (ESC)-like morphology are selected for further propagation, characterization, and low temperature storage. Generally, ESC morphology is compact colonies having a high nucleus to cytoplasm ratio, prominent nucleoli, sharp adages, and flat colonies. In some examples, the medium is changed daily, and ESC colonies are split about every 5-7 days manually or by disaggregation in collagenase IV (for example, at about 1 mg/ml and about 37° C. for about 2-3 minutes; Invitrogen), and collected cells are replated onto dishes with fresh feeder layers. Cultures are maintained at about 37° C. with about 3% CO₂, about 5% O₂, and about 92% N₂. In another alternative, serum-free media is used.

Homozygous PSCs can then be isolated, and PSCs can be maintained in vitro using standard procedures. In one embodiment, primate PSCs are isolated on a confluent layer of fibroblast in the presence of ESC medium. In one example, to produce a feeder layer, xenogeneic embryonic fibroblasts are obtained from 14-16-day-old fetuses from outbred mice (such as CF1, available from SASCO), but other strains may be used as an alternative. Alternatively, human fibroblasts obtained from adult skin or cells obtained from TSC-derived fibroblasts can be employed. In another embodiment, tissue culture dishes treated with about 0.1% gelatin (type I; Sigma) can be utilized. Unlike mouse PSCs, human PSCs (hPSCs) do not express the stage-specific embryonic antigen stage-specific embryonic antigen (SSEA)-1, but express SSEA-4, which is another glycolipid cell surface antigen recognized by a specific monoclonal antibody (see, for example, Amit et al., Devel. Biol. 227:271-278, 2000).

ICM-dissociated cells can be plated on feeder layers in fresh medium and observed for colony formation. Colonies demonstrating ESC morphology are individually selected and split again as described above. Resulting PSCs are then routinely split by mechanical methods every six days as the cultures become dense. Early passage cells are also frozen and stored in liquid nitrogen.

Homozygous PSCs as well as transplantable cells can be produced and can be karyotyped with, for example, a standard G-banding technique (such as by the Cytogenetics Laboratory of the University of Wisconsin State Hygiene Laboratory, which provides routine karyotyping services) and compared to published karyotypes for the primate species.

In other embodiments, immunosurgical isolation of the ICM is not utilized. Thus, the blastocysts are cultured directly without the use of any immunosurgical techniques. Isolation of primate PSCs from blastocysts, including humans, would follow a similar procedure, except that the rate of development of TSCs to blastocyst can vary by a few days between species, and the rate of development of the cultured ICMs will vary between species. For example, eight days after fertilization, rhesus monkey embryos are at the expanded blastocyst stage, whereas human embryos reach the same stage 5-6 days after fertilization. Because other primates also vary in their developmental rate, the timing of the initial ICM split varies between primate species, but the same techniques and culture conditions will allow for ESC isolation (see U.S. Pat. No. 6,200,806, which is incorporated herein by reference, for a complete discussion of primate ES cells and their production). Culture conditions described above can also be used for the culture of PSCs from blastocysts.

Conditions for culturing human TSCs obtained by conventional protocols from fertilized oocytes to blastocysts have been described (see Bongso et al., Hum Reprod. 4:706-713, 1989). In some embodiments, co-culturing of human TSCs with human oviductal cells results in the production of high quality blastocyst. Human ICM from blastocysts grown in cellular co-culture or in media that eliminates the feeder cell layer requirement allows for isolation of human PSCs with the same procedures described above for non-human primates.

TSCs can be used to generate extraembryonic cells, such as trophectoderm, that are of use in cell culture. In one embodiment, the use of autologous cells (e.g., trophectoderm) as feeder cells can be helpful to generate stem cells that, in turn, have the capacity to differentiate into differentiated organ-specific cells. In other embodiments, the use of allogeneic feeder cells obtained by using culturing totipotent stem cells in such a manner to allow the generation of such feeder layer component is useful to avoid xeno-contamination and, thus, allow for easier FDA approval of the differentiated cells cultured thereupon for therapeutic purposes.

Homozygous pluripotent stem cells can (PSCs) also be produced. The TSCs can then be cultured as described above to produce PSCs and multipotent stem cells (MPSCs). Alternatively, multipotent stem cells (isolated from a subject or from a cell line) can be treated using the disclosed methods. Following treatment, a therapeutically effective amount of the resultant homozygous multipotent cells can be used for transplantation into a subject of interest.

The homozygous primate PSCs produced using the methods disclosed herein are useful for the generation of cells of desired cell types. In some embodiments, the PSCs are used to derive mesenchymal, neural, and/or hematopoietic stem cells. In other embodiments, the PSCs are used to generate cells, including, but not limited to, pancreatic, liver, bone, epithelial, endothelial, tendon, cartilage, and muscle cells and their progenitor cells. In alternative embodiments, mesenchymal, neural, and/or hematopoietic stem cells are used.

Homozygous cells produced using the methods disclosed herein can be transplanted into a subject. In an embodiment, cells matched at one or more MHC loci to the treated individual. In an embodiment, the cells are cultured in media free of serum. In an embodiment, the cells have not been cultured with xenogeneic cells (e.g., non-human fibroblasts, such as mouse embryonic fibroblasts). Methods for treating disease are provided that include transplanting homozygous cells derived from PSCs or using homozygous somatic cells directly prepared using the disclosed methods.

Thus, transplantable homozygous cells can be administered to an individual in need of one or more cell types to treat a disease, disorder, or condition. Examples of diseases, disorders, or conditions that may be treated or prevented include neurological, endocrine, structural, skeletal, vascular, urinary, digestive, integumentary, blood, immune, auto-immune, inflammatory, kidney, bladder, cardiovascular, cancer, circulatory, hematopoietic, metabolic, reproductive, and muscular diseases, disorders, and conditions. In some embodiments, a hematopoietic stem cell is used to treat cancer. In some embodiments, these cells are used for reconstructive applications, such as for repairing or replacing tissues or organs.

The TSCs and PSCs described herein can be used to generate multipotent stem cells or transplantable cells. Multipotent stem cells can also be treated directly using the presently claimed methods. The cells can be bone marrow stem cells, hematopoietic stem cells, mesenchymal stem cells, intestinal stem cells, neuronal stem cells, or dental stem cells.

In an example, the homozygous cells are mesenchymal stem cells. Mesenchymal stem cells give rise to a very large number of distinct tissues (Caplan, J. Orth. Res 641-650, 1991). Mesenchymal stem cells capable of differentiating into bone, muscles, tendons, adipose tissue, stromal cells, and cartilage have also been isolated from marrow (Caplan, J. Orth. Res., 641-650, 1991). U.S. Pat. No. 5,226,914 describes an exemplary method for isolating mesenchymal stem cells from bone marrow. In some examples, homozygous epithelial progenitor cells or keratinocytes can be generated for use in treating conditions of the skin and the lining of the gut (Rheinwald, Meth. Cell Bio. 21A:229, 1980). The methods also can be used to produce liver precursor cells (see PCT Publication No. WO 94/08598) or kidney precursor cells (see Karp et al., Dev. Biol. 91:5286-5290, 1994). The methods can be used to produce homozygous inner ear precursor cells (see Li et al., TRENDS Mol. Med. 10: 309, 2004).

In embodiments, the methods include administering one or more cells (e.g., homozygous transplantable cells, such as pluripotent or multipotent stem cells) to a subject (e.g., a primate or human subject) that were produced by propagating cells in vitro produced using the disclosed methods. Generally, a therapeutically effective amount of homozygous cells is administered to an individual. The cells can be administered in a pharmaceutical carrier. The pharmaceutically acceptable carriers of use are conventional. For example, Remington's Pharmaceutical Sciences, by E. W. Martin, Mack Publishing Co., Easton, Pa., 15^{th} Edition, 1975, describes compositions and formulations suitable for pharmaceutical delivery of the cells herein disclosed. In general, the nature of the carrier will depend on the particular mode of administration being employed. For instance, parenteral formulations usually comprise injectable fluids that include pharmaceutically and physiologically acceptable fluids, such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH-buffering agents, and the like, for example, sodium acetate or sorbitan monolaurate.

The individual can be any subject of interest. Suitable subjects include those subjects that would benefit from proliferation of cells derived from stem cells or precursor cells (e.g., a primate or human subject in need of therapy). In some examples, the subject is in need of proliferation of cardiac cells. For example, the individual can have cardiomyopathy and/or hypercholesterolemia. In some examples, the individual is in need of proliferation of neuronal precursor cells and/or glial precursor cells.

In some embodiments, the individual has a neurodegenerative disorder or an ischemic event, such as a stroke. Specific, non-limiting examples of a neurodegenerative disorder are Alzheimer's disease, pantothenate kinase-associated neurodegeneration, Parkinson's disease, Huntington's disease (Dexter et al., Brain 114:1953-1975, 1991), HIV encephalopathy (Miszkziel et al., Magnetic Res. Imag. 15:1113-1119, 1997), and amyotrophic lateral sclerosis. Suitable individuals also include those subjects that are aged, such as individuals who are at least about 65, at least about 70, at least about 75, at least about 80, or at least about 85 years of age. In additional examples, the individual can have a spinal cord injury, Batten's disease, or spina bifida. In further examples, the individual can have hearing loss, such as a subject who is deaf, or can be in need of the proliferation of stem cells from the inner ear to prevent hearing loss.

In some examples, the homozygous cell can be a neuronal cell (e.g., produced using the methods disclosed herein, such as using neuronal stem cells). The volume of a cell suspension, such as a neuronal cell suspension, administered to a subject will vary depending on the site of implantation, treatment goal and amount of cells in solution. Typically, the amount of cells administered to a subject will be a therapeutically effective amount. For example, where the treatment is for Parkinson's disease, transplantation of a therapeutically effective amount of cells will typically produce a reduction in the amount and/or severity of the symptoms associated with that disorder (e.g., rigidity, akinesia, and gait disorder). In one example, a severe Parkinson's patient needs at least about 100,000 surviving dopamine cells per grafted site to have a substantial beneficial effect from the transplantation. As cell survival is low in brain tissue transplantation in general (5-10%), at least 1 million cells are administered, such as transplantation from about 1 million to about 4 million dopaminergic neurons. In one embodiment, the cells are administered to the subject's brain. The cells can be implanted within the parenchyma of the brain in the space containing cerebrospinal fluids, such as the sub-arachnoid space or ventricles, or extaneurally. Thus, in one example, the cells are transplanted to regions of the subject that are not within the central nervous system or peripheral nervous system, such as the celiac ganglion or sciatic nerve. In another embodiment, the cells are transplanted into the central nervous system, which includes all structures within the dura mater. Injections of neuronal cells can generally be made with a sterilized syringe having an 18-21 gauge needle. Although the exact size needle will depend on the species being treated, the needle should not be bigger than 1 mm diameter in any species. Those of skill in the art are familiar with techniques for administering cells to the brain of a subject.

Cells produced by the methods disclosed herein, such as homozygous TSCs and PSCs, are also of use for testing agents of interest, such as to determine if an agent affects differentiation or cell proliferation. For example, homozygous TSCs or PSCs are contacted with the agent, and the ability of the cells to differentiate or proliferate is assessed in the presence and the absence of the agent. Thus, cells produced by the methods disclosed herein can also be used to screen pharmaceutical agents to select for agents that affect specific human cell types, such as agents that affect neuronal cells. Cells produced by the methods disclosed herein can also be used to screen one or more agents to select those that affect differentiation. The test compound can be any compound of interest, including chemical compounds, small molecules, polypeptides, or other biological agents (for example antibodies or cytokines). In several examples, a panel of potential agents are screened, such as a panel of cytokines or growth factors.

Methods for preparing a combinatorial library of molecules that can be tested for a desired activity are well-known in the art and include, for example, methods of making a phage display library of peptides, which can be constrained peptides (see, for example, U.S. Pat. Nos. 5,622,699, 5,206,347; Scott and Smith, Science, 249:386-390, 1992; Markland et al., Gene, 109:13 -19, 1991); a peptide library (U.S. Pat. No. 5,264,563); a peptidomimetic library (Blondelle et al., Trends Anal Chem., 14:83-92, 1995); a nucleic acid library (O'Connell et al., Proc. Natl Acad. Sci., USA 93:5883-5887, 1996; Tuerk and Gold, Science 249:505-510, 1990; Gold et al., Ann. Rev. Biochem. 64:763-797, 1995); an oligosaccharide library (York et al., Carb. Res. 285:99-128, 1996; Liang et al., Science 274:1520-1522, 1996; Ding et al., Adv. Expt. Med. Biol. 376:261-269, 1995); a lipoprotein library (de Kruif et al., FEBS Lett. 3 99:23 2-23 6, 1996); a glycoprotein or glycolipid library (Karaoglu et al., J Cell Biol. 130.567-577, 1995); or a chemical library containing, for example, drugs or other pharmaceutical agents (Gordon et al., J Med. Chem. 37.1385-1401, 1994; Ecker and Crooke, BioTechnology 13:351-360, 1995). Polynucleotides can be particularly useful as agents that can alter a function in pluripotent or totipotent cells because nucleic acid molecules having binding specificity for cellular targets, including cellular polypeptides, exist naturally and because synthetic molecules having such specificity can be readily prepared and identified (see, for example, U.S. Pat. No. 5,750,342).

In one embodiment, for a high throughput format, homozygous TSCs, PSCs, or MPSCs produced by the methods disclosed herein can be introduced into wells of a multiwell plate or of a glass slide or microchip and can be contacted with the test agent. Generally, the cells are organized in an array, particularly an addressable array, such that robotics conveniently can be used for manipulating the cells and solutions as well as for monitoring the cells, particularly with respect to the function being examined An advantage of using a high throughput format is that a number of test agents can be examined in parallel, and, if desired, control reactions also can be run under identical conditions as the test conditions. As such, the methods disclosed herein provide a means to screen one, a few, or a large number of test agents to identify an agent that can alter a function of the cells, for example, an agent that induces the cells to differentiate into a desired cell type or that prevents spontaneous differentiation, for example, by maintaining a high level of expression of regulatory molecules.

The cells are contacted with test compounds sufficient for the compound to interact with the cell. When the compound binds a discrete receptor, the cells are contacted for a sufficient time for the agent to bind its receptor. In some embodiments, the cells are incubated with the test compound for an amount of time sufficient to affect phosphorylation of a substrate. In some embodiments, cells are treated in vitro with test compounds at 37° C. in a 5% CO₂ humidified atmosphere. Following treatment with test compounds, cells are washed with Ca²+- and Mg²+-free PBS, and the total protein is extracted as described (Haldar et al., Cell Death Diff. 1:109-115, 1994; Haldar et al., Nature 342:195-198, 1989; Haldar et al., Cancer Res. 54:2095-2097, 1994). In additional embodiments, serial dilutions of test compound are used.

The disclosure is illustrated by the following non-limiting Examples.

### EXAMPLES

CRISPR/Cas is a versatile tool for recognizing specific genomic sequences and inducing double-strand breaks (DSBs) (Hsu et al., Cell, 157:1262-1278, 2014; Mali et al., Science, 339:823-826, 2013; Kim et al., Genome research, 24:1012-1019, 2014; Cong et al., Science, 339:819-823, 2013). DSBs are then resolved by endogenous DNA repair mechanisms, preferentially using a non-homologous end-joining (NHEJ) pathway. NHEJ is inappropriate for gene correction applications because it introduces additional mutations in the form of insertions or deletions at the DSB site, commonly referred to as indels. In a few cases, however, targeted cells activate an alternative DNA repair pathway called homology-directed repair (HDR) that rebuilds the DSB site using the non-mutant homologous chromosome or a supplied exogenous DNA molecule as a template leading to actual correction of the mutant allele (Lin et al., Elife, 3:e04766, 2014; Wu et al., Cell stem cell, 13:659-662, 2013). At present, CRISPR/Cas9 is predominantly used to introduce mutations and in the generation of gene knockouts utilizing intrinsic NHEJ. Because HDR efficiency is relatively low, applications of genome editing for gene therapy have been limited (Mali et al., 2013; e.g., Suzuki et al., Nature Scientific Reports, 4:7621, 2014, and WO 2016/097751 A1, injecting unfertilized mouse metaphasell (MII) oocytes with Cas9 cRNA, gRNA, and sperm to enable editing of transgenic and native alleles, and Hashimoto et al., Developmental Biology, 418: 1-9, 2016, using electroporation to introduce Cas9 protein and sgRNA into mouse zygotes prior to first replication to enable non-mosaic mutants).

Human gamete and embryo DNA repair mechanisms activated in response to the CRISPR/Cas9-induced DSBs were investigated. Experiments were performed targeting the heterozygous four-base-pair (bp) deletion in the MYBPC3 gene in human zygotes introduced by heterozygous carrier sperm, while oocytes collected from healthy donors provided the wild-type allele. By accurate analysis of cleaving embryos at the single cell level, high targeting efficiency and specificity in preselected CRISPR/Cas9 constructs were shown. Moreover, DSBs in the mutant paternal MYBPC3 gene were preferentially repaired using the wild-type oocyte allele as a template, suggesting an alternative, germline-specific DNA repair response. Mechanisms responsible for mosaicism in embryos were also investigated with a proposed solution to minimize its occurrence, namely the co-injection of sperm and CRISPR/Cas9 components into metaphase 2 (MII) oocytes.

CRISPR/Cas9 was used for correction of an exemplary heterozygous MYBPC3 mutation in human preimplantation embryos with precise targeting accuracy and dramatically high homology-directed repair (HDR) efficiency by activating an endogenous, germline-specific DNA repair response. Induced double-strand breaks at the mutant paternal allele were predominantly repaired using the homologous wild-type maternal gene instead of a synthetic DNA template. By modulating the cell cycle stage at which CRISPR/Cas9 was introduced, mosaicism in cleaving embryos was avoided, resulting in a high yield of homozygous embryos carrying the wild-type MYBPC3 gene and without evidence of off-target mutations. These results show that many barriers to human germline gene editing can be overcome and support efficient, accurate, and safe correction of heritable mutations in human embryos. Germline gene correction represents an alternative to preimplantation genetic diagnosis and has the advantage of rescuing a substantial portion of mutant human embryos, thus, increasing the number of embryos available for transfer.

### Example 1

### Methods

This example describes the methods and materials used in Examples 1-6.

Regulations for Research on Human Gametes and Embryos: The regulatory framework surrounding the use of human gametes and embryos for this research was based on the guidelines set by the Oregon Health & Science University (OHSU) Stem Cell Research Oversight Committee (OSCRO). The OSCRO established (in 2008) a policy and procedural guidelines formally defining the use of human embryos and their derivatives at OHSU, which were informed by the National Academy of Sciences' Guidelines. These policies and guidelines permitted procurement of gametes and embryos for research purposes, creation of human embryos specifically for research, genetic manipulation of human gametes/embryos, creation of human embryonic stem cell lines, and molecular analysis. Together, OSCRO and the OHSU Institutional Review Board (IRB) worked concurrently to review and monitor applications for research studies involving human embryos at OHSU.

Human embryo and embryonic stem cell research policies and principles at OHSU were investigated over the course of a decade, informed by the NAS guidelines, and, subsequently, affirmed by new guidelines released in 2015 by the Hinxton Group and the International Society for Stem Cell Research (ISSCR) as well as by recommendations released in 2017 by the NAS and National Academy of Medicine joint panel on human genome editing.

As part of the review process, OHSU convened additional ad-hoc committees to evaluate the scientific merit and ethical justification of the proposed study: the OHSU Innovative Research Advisory Panel (IRAP) and a Scientific Review Committee (SRC).

Ethical Review: While international discussions were in their infancy, the OHSU Innovative Research Advisory Panel (IRAP) committee was tasked with deliberating on the ethical considerations of utilizing gene correction technology in human embryos for basic research at OHSU. The committee was composed of eleven members from internal and external sources: a lay member, a clinical ObGyn physician, three bioethicists, an OHSU Institutional Ethics committee member, three former OSCRO members, a clinical geneticist, and a clinician. Upon completion of the review, the IRAP recommended allowing this research “with significant oversight and continued dialogue, the use of gene correction technologies in human embryos for the purpose of answering basic science questions needed to evaluate germline gene correction prior to the use in human models” at OHSU.

Study Oversight: The established track record of the study team to uphold strict confidentiality and regulatory requirements paved the way for full OHSU IRB study approval in 2016, contingent upon strict continuing oversight, which includes a phased scientific approach to evaluate the safety and efficacy of germline gene correction in human pre-implantation embryos, external bi-annual monitoring of all regulatory documents regarding human subjects, bi-annual Data Safety Monitoring Committee (DSMC) review, and annual continuing review by the OHSU IRB. The DSMC consists of four members: a lay member, an ethicist, a geneticist, and a reproductive endocrinologist, whose purview includes monitoring all future uses of materials generated by this protocol.

Informed Consent: The robust regulatory framework set forth by OHSU clearly specified that informed consent could only be obtained if perspective donors were made aware of the sensitive nature of the study. This excerpt from the consent form clearly presented the scientific rationale of the study. Additionally, consent form language clearly stated genetic testing would be conducted in addition to creation of preimplantation embryos and embryonic stem cell lines for in vitro analyses and stored for future uses. Incidental findings, genetic information potentially important to the donors' healthcare, are a possible outcome when engaging in this type of research. Informed consent documents provided the donor with the option to receive this information or not. Written informed consent was obtained prior to all study-related procedures.

Study Participants: Healthy gamete donors were recruited locally via print and web-based advertising. A sperm donor with a heterozygous MYBPC3 mutation was identified by OHSU Knight Cardiovascular Institute physicians and referred to the research team.

Controlled Ovarian Stimulation: Research oocyte donors were evaluated prior to study inclusion as previously reported; standard WF protocols and procedures for ovarian stimulation were as described previously (Tachibana et al., Nature, 493:627-631, 2013). Oocyte donation cycles were managed by OHSU Fertility physicians Immediately following oocyte retrieval, recovered gametes were transferred to the research laboratory. All study-related procedures took place at the OHSU Center for Embryonic Cell and Gene Therapy. Following oocyte retrieval, cumulus-oocyte complexes (COCs) were treated with hyaluronidase to disaggregate cumulus and granulosa cells. Mature metaphase II (MII) oocytes were placed in Global Medium (LifeGlobal, IVFonline) supplemented with 10% SSS (Global 10%) at 37° C. in 6% CO₂ and covered with tissue culture oil (Sage WF, Cooper Surgical).

Compensation: All research donors were compensated for their time, effort, and discomfort associated with the donation process at rates similar to gamete donation for fertility purposes.

Intracytoplasmic Sperm Injection (ICSI): MB oocytes were placed into a 50 μL micromanipulation droplet of HTF with HEPES 10% medium. The droplet was covered with tissue culture oil. The dish was then mounted on the stage of an inverted microscope (Olympus IX71) equipped with a stage warmer (see tokaihit.com) and Narishige micromanipulators. Oocytes were fertilized by intracytoplasmic sperm injection (ICSI) using frozen/thawed sperm. Fertilization was determined approximately 18 hours after ICSI by noting the presence of two pronuclei and the second polar body extrusion.

CRISPR/Cas9 Injection into Zygote or Oocytes: For S-phase injections, zygotes were collected 18 hours after ICSI, placed into a micromanipulation drop, and injected into a cytoplasm with a CRISPR/Cas9 mixture containing Cas9 protein (200 ng/μL), sgRNA (100 ng/μL), and ssODN (200 ng/μL). Injected zygotes were cultured in Global 10% medium at 37° C. in 6% CO₂, 5% O₂, and 89% N₂ for up to 3 days to the 4-8 cell stage. For M-phase injections, CRISPR/Cas9 was co-injected with sperm during ICSI. A single sperm was first washed in a 4 μL drop of mixture containing Cas9 protein, sgRNA, and ssODN, as described above.

Blastomere isolation, whole genome amplification, and Sanger sequencing: Zonae pellucidae from the 4-8 cells stage embryos were removed by brief exposure to acidic Tyrode solution (NaCl 8 mg/mL, KCl 0.2 mg/mL, CaCl₂.2H₂O 2.4 mg/mL, MgCl₂.6H₂O 0.1 mg/mL, glucose 1 mg/mL, PVP 0.04 mg/mL). Zona-free embryos were briefly (30 sec) exposed to trypsin solution (0.15% in EDTA containing Ca- and Mg-free PBS) before manual disaggregation into single blastomeres with a small bore pipette. A total of 830 blastomeres were isolated from 131 embryos, including 19 from control, 54 from zygote-injected, and 58 from M-phase-injected groups. Individual blastomeres were transferred into 0.2 ml PCR tubes containing 4μL PBS and placed into −80° freezer until further use. Whole-genome amplification from individual blastomeres was performed using a REPLI-s Single Cell Kit (Qiagen). Amplified DNA was diluted 1/100, and the on-target region was amplified by PCR using a PCR Platinum SuperMix High Fidelity Kit (Life Technologies) with the primer set F 5′-CCCCCACCCAGGTACATCTT-3′ (SEQ ID NO: 40) and R 5′-CTAGTGCACAGTGCATAGTG-3′ (SEQ ID NO: 41). PCR products of 534 bp were purified, Sanger sequenced, and analyzed by Sequencher v5.0 (GeneCodes). Of the 830 blastomeres, 730 (88%) resulted in successful libraries and produced PCR products for MYBPC3, while the remaining 100 blastomeres (12%) failed to generate PCR products and were excluded from the study.

iPSC Derivation and Transfection with CRISPR/Cas9: Patient iPSCs were derived from skin fibroblasts with a CytoTune-iPS Reprogramming Kit (Life Technologies) according to the manufacturer's protocol. Cell lines were cultured in mTeSR1 medium (STEMCELL technology) at 37° C. in a humidified atmosphere containing 5% CO₂. To test CRISPR/Cas9, 2×10⁵ iPSCs were dissociated into single cells (Accutase from STEMCELL technology, or TrypLe from Invitrogen). For the CRISPR/Cas9-1 construct, a Cas9 expression plasmid (p3 s-Cas9HC, 2.4 μg), sgRNA expression plasmid (pU6-sgRNA, 1.6 μg), and ssODN-1 (100 pmol, IDT) were transfected using an Amaxa P3 Primary Cell 4D-Nucleofector Kit (Program CB-150) according to the manufacturer's protocol. Three days after transfection, approximately 5,000 cells were plated onto a Matrigel-coated culture dish and cultured for clonal propagation and individual clone selection. For the CRISPR/Cas9-2 construct, 15 μg of Cas9 expression plasmid (pCAG-1BPNLS-Cas9-1BPNLS), 15 μg of sgRNA expression plasmids (pCAGmCherry-MYBPC3gRNA), and 30 μg of ssODN-2 were co-transfected by electroporation using the BioRad Gene Pulser II (a single 320-V, 200-μF pulse at room temperature) with a 0.4-cm gap cuvette. Cells were plated at high density on 6-well plates coated with Matrigel. Two to three days after electroporation, iPSCs were harvested and subjected to clonal selection. All cell lines were negative for mycoplasma contamination. For direct comparisons of CRISPR-Cas9-1 and CRISPR-Cas9-2, Cas9 RNP complexes composed of the recombinant Cas9 protein (15 μg) and sgRNA (20 μg) were co-transfected with ssODN-1 (50-200 pmol, IDT) into iPSCs (2×105 cells) via electroporation as described above. Three days after transfection, indel and HDR efficiencies were analyzed by targeted deep sequencing .

Recombinant Cas9 protein and in vitro transcription of sgRNA: Recombinant Cas9 protein was purchased from ToolGen, Inc. The sgRNA was synthesized by in vitro transcription using T7 polymerase (New England Biolabs), as described previously (Kim et al., Nature communications, 5:3157, 2014). In brief, sgRNA templates were generated by annealing and extension of two oligonucleotides. Next, in vitro transcription was performed by incubating sgRNA templates with T7 RNA polymerase supplemented with NTPs (Jena Bioscience) and RNase inhibitor (New England Biolabs) overnight at 37° C. In vitro transcribed RNA was then treated with DNase I (New England Biolabs) for 30 min at 37° C. and purified using a MinElute Cleanup kit (Qiagen).

Targeted deep sequencing, genomic DNA cleavage, whole genome, and Digenome sequencing: To analyze HDR and NHEJ frequencies, on-target and off-target regions were amplified using Phusion polymerase (New England Biolabs). PCR amplicons were subjected to paired-end sequencing using Illumina Miniseq. A Cas-analyzer was used for analyzing indel and HDR frequencies (Bae et al., Bioinformatics, 30:1473-1475, 2014; Park et al., Bioinformatics, 33:286-288, 2017). Genomic DNA was isolated from patient iPSCs using a DNeasy Tissue Kit (Qiagen). Digenome-seq was performed according to previous publications (Kim et al., Nature methods, 12:237-243, 231 p following 243, 2015; Kim et al., Genome research, 26:406-415, 2016). In brief, 20 μg of genomic DNA was cleaved by incubating recombinant Cas9 protein (16.7 μg) and in vitro transcribed sgRNA (12.5 μg) in 1X NEB buffer 3.1(100 mM NaCl, 50 mM Tris-HCl, 10 mM MgCl₂, 100 μg/ml BSA, pH 7.9) at 37° C. for 3 hr. Cas9- and sgRNA-treated genomic DNA was treated with 50 μg/ml of RNase A (Sigma Aldrich) at 37° C. for 30 min and purified with a DNeasy Tissue Kit (Qiagen). Whole-genome and Digenome sequencing were performed as described previously (id.). In brief, 1 ug of genomic DNA was fragmented and ligated with adaptors using TruSeq DNA libraries. DNA libraries were subjected to whole-genome sequencing using an Illumina HiSeq X Ten Sequencer at Macrogen (30X to 40X). The sequence file was aligned to the human reference genome hg19 from UCSC with the following mapping program and parameters using Isaac aligner: Base quality cutoff, 15; Keep duplicate reads, yes; Variable read length support, yes; Realign gaps, no; and Adaptor clipping, yes (adaptor: AGATCGGAAGAGC* (SEQ ID NO: 42), *GCTCTTCCGATCT (SEQ ID NO: 43). In vitro DNA cleavage sites were identified computationally using a DNA cleavage scoring system described previously (Raczy et al., Bioinformatics, 29:2041-2043, 2013; Kim et al., 2016). Indel frequencies of 23 genomic loci with DNA cleavage score above the 0.1 cutoff value were individually examined in individual blastomeres by targeted deep sequencing.

Analysis of off-target effects in CRISPR-Cas9-injected human embryos by WGS: WGS was performed using an Illumina HiSeq X Ten sequencer with a sequencing depth of 30× to 40× (Macrogen, South Korea). Sequences from each blastomere were processed to determine the total variants using the Isaac variant calling program (Raczy et al., 2013). Annotated variants, including dbSNPs and all novel SNPs (substitution changes), were filtered out, and novel indel sites were identified. Cas-OFFinder (Bae et al., 2014) was used to extract potential off-target sequences that differed from the on-target sequence by up to 7 nucleotide mismatches or up to 5 nucleotide mismatches with a DNA bulge of up to 2 nucleotides. Indel sites found in each blastomere were compared to homologous sites identified by Cas-OFFinder , and potential off-target sites were identified. Potential off-target sites were then excluded; potential off-target sites were found in intact control embryos. Finally, whether CRISPR-Cas9 caused any of these potential off-target sites was assayed by inspecting sequences with Integrative Genomics Viewer (Robinson et al., 2011).

Whole-exome sequencing and data analyses: Whole-exome sequencing (WES) was performed using genomic DNA isolated from the peripheral blood of the sperm donor and two egg donors (egg donor 1 and egg donor 2) and ES cells derived from individual human embryos (ES-WT1, ES-Mutl, and ES-C1 were from egg donor 1; ES-WT2 and ES-WT3 were from egg donor 2). ES-WT1, ES-WT2 and ES-WT3 were from treated wild-type embryos. ES-C1 was from an untreated wild-type embryo. ES-Mutl was from a treated heterozygous mutant embryo. Sequencing libraries were prepared according to the instructions for Illumina library preparation. Exome capture was performed using an Agilent V5 chip. Sequencing was performed using an Illumina Hiseq 4000 platform with paired-end 101 (PE101) strategy at a depth of 100×. All sequencing data were first processed by filtering adaptor sequences and removing low quality reads or reads with a high percentage of N bases using SOAPnuke (1.5.2) software (soap.genomics.org.cn) developed by BGI, and clean reads were generated for each library. Clean data were paired-end aligned using the Burrows-Wheeler Aligner (BWA) program version 0.7.12 to the human genome assembly hg19. Duplicate reads in alignment BAM files were identified using MarkDuplicates in Picard (1.54). The alignment results were processed using the RealignerTargetCreator, IndelRealigner, and BaseRecalibrator modules in GATK (3.3.0). Variant detection was performed using HaplotypeCaller tool in GATK. SNV and indel information was extracted and filtered by VQSR in GATK and annotated by AnnoDB (v3). The guide sequence (GGGTGGAGTTTGTGAAGTAT, SEQ ID NO: 3) was aligned to the human genome assembly hg19 to identify potential off-target sites the full sensitive aligner Batmis (V3.00), allowing a maximum of five mismatches globally and a maximum of two mismatches in the core region (12 bp adjacent to the PAM site). Inherited variants from parents and all novel SNPs (substitution changes) were filtered out, and novel indels located within the off-target site plus flanking 20-bp region were defined as off-target variants.

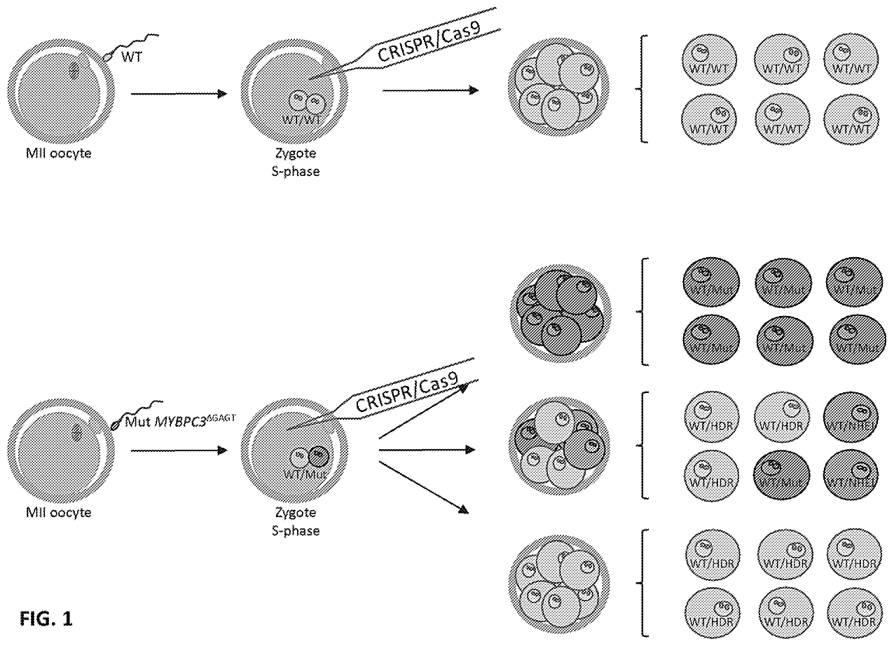

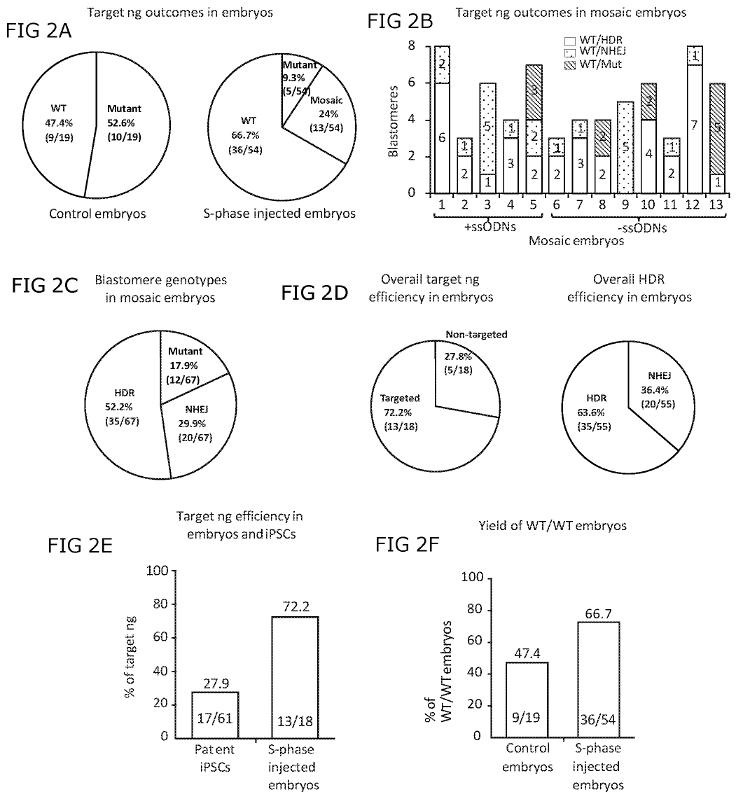

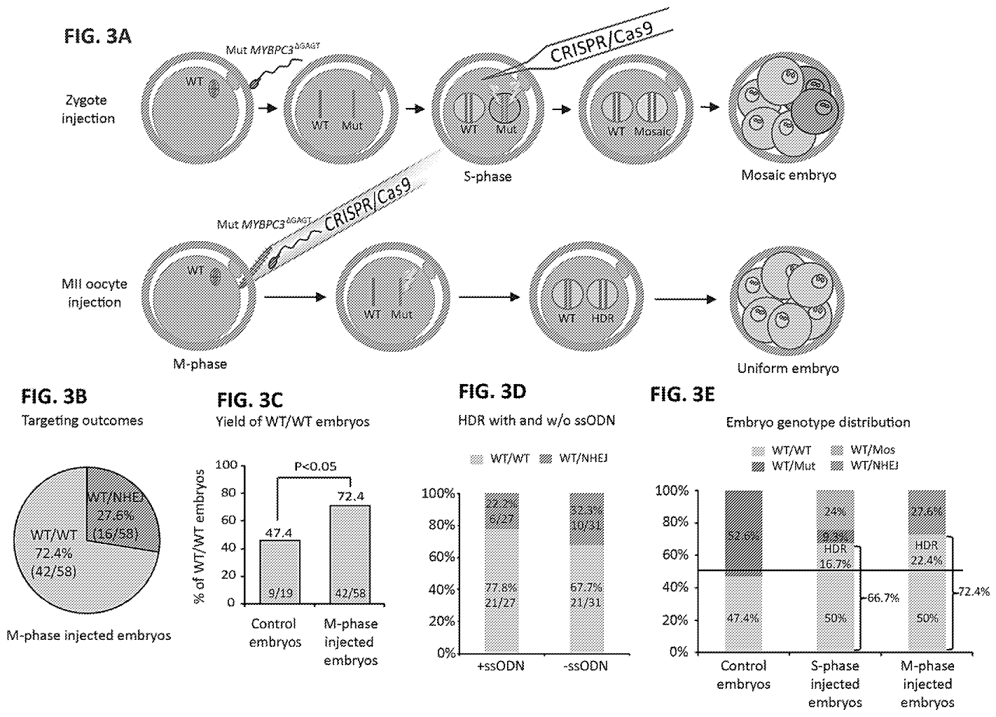

Statistical Analyses: A one-tailed Fisher's test was used for the comparisons in FIG. 2F, FIG. 3C, and FIG. 5E. A contingency table was used for the comparison in FIGS. 1F and 2C-2D. A P-value less than 0.05 was considered significant.

### Example 2

### Subject with a Heterozygous MYBPC3^{ΔGAGT} Deletion and Selection of CRISPR/Cas9 Constructs

An adult male patient with well-documented HCM caused by a heterozygous dominant 4 bp GAGT deletion (g.9836_9839 del) in exon 16 of the MYBPC3 gene and currently managed with an implantable cardioverter defibrillator agreed to donate skin and semen samples. Skin fibroblast cultures were expanded and used to generate heterozygous patient iPSCs as described previously (Kang et al., Cell Stem Cell, 18:625-636, 2016). Two small guide RNA (sgRNA)-Cas9 constructs were designed, targeting this specific MYBPC3^{ΔGAGT} deletion (FIGS. 5A-5B) along with two exogenous single-stranded oligodeoxynucleotide (ssODN) templates encoding homology arms to the targeted region (FIGS. 5A-5F; Cho et al., Nature biotechnology, 31:230-232, 2013; Kim and Kim, Nat Rev Genet, 15:321-334, 2014; Jinek et al., Science, 337:816-821, 2012). To differentiate from the WT allele, two synonymous single nucleotide substitutions were introduced into each ssODN template. ssODN-2 nucleotide substitutions provided an additional restriction enzyme (BstBI) recognition site (FIGS. 5A-5B).

The efficacy and specificity of each construct was tested by transfecting patient iPSCs. Cells were electroporated together with ssODN, Cas9, and sgRNA expression plasmids. The cells were then subcloned, and the targeted region for each clone was analyzed by sequencing (FIG. 5C). Of 61 iPSC clones transfected with CRISPR/Cas9-1, 44 (72.1%, 44/61) were not targeted, as evidenced by the presence of both intact WT and intact mutant (Mut) alleles (FIGS. 5D-5E). Among the targeted clones, 10 of 17 (58.8%) were repaired by NHEJ and contained various indels adjacent to the mutation site. The remaining 7 clones were repaired by HDR using ssODN-1, as demonstrated by the presence of the marker nucleotide substitutions. Thus, the total targeting efficiency for CRISPR/Cas9-1 was 27.9% (17/61). Among the targeted clones, only 41.2% (7/17) were repaired by HDR (FIGS. 5E). The targeting efficiency with CRISPR/Cas9-2 (13.1%; 23/175) and HDR was considerably lower at 13% (3/23). Of note, among the 3 HDR-repaired iPSC clones, 2 were repaired using the ssODN-2 template, while the third clone contained intact WT sequences in both alleles (FIGS. 5D-5E), indicating HDR using the WT allele.

In all iPSC clones transfected with either construct, the WT allele remained intact, demonstrating high fidelity of sgRNAs. A direct comparison of CRISPR-Cas9-1 and CRISPR-Cas9-2 in patient iPSCs transfected with preassembled Cas9 ribonucleoproteins (RNPs) was also performed. Targeted deep sequencing demonstrated that CRISPR-Cas9-1 had higher HDR efficiency (FIG. 5F). On-target mutations were not detected in wild-type embryonic stem (ES) cells (H9) carrying both wild-type MYBPC3 alleles, demonstrating a high specificity for CRISPR-Cas9-1. Based on these outcomes, CRISPR/Cas9-1 (hereafter referred to as CRISPR/Cas9) with a higher efficiency of HDR-based gene correction was selected for subsequent studies.

### Example 3

### HDR Efficiency in Human Heterozygous MYBPC3^{ΔGAGT} zygotes injected with CRISPR/Cas9

Targeting outcomes were evaluated in human zygotes. Zygotes were produced by fertilizing healthy donor oocytes with sperm from a patient carrying a heterozygous MYBPC3 mutation. Because direct introduction of Cas9 protein is more efficient than using a plasmid, recombinant Cas9 protein microinjection was adopted, employing a mixture of sgRNA, Cas9 protein, and ssODN DNA into the cytoplasm of pronuclear stage zygotes 18 hrs after fertilization (Kim et al., 2014; Aida et al., Genome biology, 16:87, 2015). Injected zygotes along with intact controls were cultured for 3 days before each embryonic blastomere was isolated and individually analyzed by sequencing (FIG. 1). Cytoplasmic microinjection of the Cas9-sgRNA was confirmed visually and shown to be efficient with a 97% zygote survival rate (68/70) and development rates comparable to controls.

Sanger sequencing of 83 individual blastomeres collected from 19 control cleaving embryos on day 3 post-fertilization revealed that 9 embryos (47.4%, 9/19) were homozygous WT (MYBPC3^{WT/WT}) and 10 (52.6%. 10/19) were heterozygous, carrying the WT maternal and mutant paternal alleles (MYBPC3^{WT/ΔGAGT}; FIG. 2A), which is the expected distribution, assuming that the heterozygous patient sperm sample contained equal numbers of mutant and WT spermatozoa with similar motilities and fertility efficiencies.

Among CRISPR/Cas9-injected human embryos, 36 (66.7%, 36/54) were uniformly homozygous for the WT allele with each blastomere containing MYBPC3^{WT/WT}, while 18 (33.3%, 18/54) were uniform or mosaic heterozygous (FIG. 2A). In this group of 18, five embryos were uniformly heterozygous with each blastomere containing the intact WT and intact mutant allele (MYBPC3^{WT/ΔGAGT}), while 13 were mosaic, each containing blastomeres carrying more than one genotype (FIG. 2A). Each mosaic embryo contained at least one heterozygous blastomere with WT and either the intact ΔGAGT deletion or the ΔGAGT deletion plus additional indels, suggesting that these embryos originated from heterozygous zygotes (MYBPC3^{WT/ΔGAGT}) that resulted from fertilization by the mutant sperm (FIG. 2B). Remarkably, a majority of the remaining sister blastomeres in all but eight mosaic embryos (numbers 1, 2, 4, 6, 7, 10, 11, and 12 in FIG. 2B) were homozygous for the WT allele (MYBPC3^{WT/WT}). Overall, 52.2% (35/67) of individual blastomeres within mosaic embryos were homozygous MYBPC3^{WT/WT} (FIGS. 2B-2C). Because these embryos originated from MYBPC3^{WT/ΔGAGT} zygotes, their blastomeres likely restored the MYBPC3 ^{ΔGAGT} deletion by HDR using the maternal WT allele as a template instead of the injected ssODNs. This conclusion was corroborated by the observation that correction occurred in blastomeres of mosaic embryos not injected with ssODNs (FIG. 2B). Among the other genotypes, four mosaic embryos (numbers 5, 8, 10, and 13 in FIG. 2B) contained blastomeres with an intact, mutant allele (MYBPC3^{WT/ΔGAGT}), but most (29.9%) also contained additional small deletions (1-20 bp long, n=16) or insertions (1 bp; n=3) adjacent to the DSB site (MYBPC3^{WT/ΔGAGT-indel}), characteristic of NHEJ. One blastomere carried a 10-bp deletion and a 5-bp insertion, while mosaic embryo #9 displayed 4 various NHEJ genotypes in its blastomeres, perhaps suggesting that targeting and NHEJ repair had occurred independently multiple times after the first zygotic division.

Based on these results, CRISPR/Cas9 targeting efficiency in human embryos was 72.2% (13/18), significantly higher than in iPSCs exposed to the same construct at 27.9% (17/61) (FIGS. 1E and 2D-2E), likely due to more efficient delivery of the CRISPR/Cas9 constructs by zygote microinjection compared with transfection in iPSCs. Even more remarkably, the majority of targeted blastomeres (63.6%, 35/55) resolved the DSBs by HDR using the WT allele, which also drastically differs from observations in iPSCs (FIGS. 1E and 2D). There was no evidence of HDR using exogenous ssODN, suggesting that HDR is exclusively guided by the WT maternal allele.

The above targeting efficacy and HDR calculations are based on mosaic embryos only; some targeted heterozygous zygotes (MYBPC3^{WT/ΔGAGT}) likely repaired the mutant allele in all their blastomeres using the WT template (MYBPC3^{WT/WT}). These HDR-repaired, uniform embryos are indistinguishable from the WT homozygous counterparts and likely increase the portion of MYBPC3^{WT/WT} embryos in the CRISPR/Cas9 injected group. Indeed, 66.7% (36/54) of injected embryos were homozygous WT/WT, a significant increase over the WT/WT yield (47.4%, 9/19) in control non-injected embryos (FIG. 2F). Similar to observations in iPSCs, no on-target mutations involving WT alleles were detected in human embryos, corroborating the specificity of the sgRNA.

In summary, these results clearly demonstrated an exceptionally high efficiency of gene targeting in human zygotes by CRISPR/Cas9, and DSBs in the mutant paternal allele were predominantly repaired through HDR. Furthermore, HDR was exclusively directed by the homologous WT allele present on the maternal chromosome. Without being bound by theory, these data suggest that human embryos employ different DNA repair mechanisms than somatic or pluripotent cells, likely reflecting evolutionary requirements for stringent control over genome fidelity in the germline.

### Example 4

### CRISPR/Cas9 Injection into MII Oocytes Eliminates Mosaicism

Mosaicism in gene-targeted human embryos is unacceptable in clinical applications. The presence even of a single mutant blastomere within a mosaic embryo would make detection by PGD problematic; therefore, molecular mechanisms responsible for mosaicism were investigated. In an analysis of targeting outcomes in the majority of mosaic, zygote-injected human embryos revealed only two different genotypes (MYBPC3^{WT/HDR} and MYBPC3^{WT/ΔGAGT-indel} or MYBPC3^{WT/HDR} and MYBPC3^{WT/ΔGAGT}; FIG. 2B). Embryos #5 and #9 were the exceptions, containing three or more genotypes. This suggests that CRISPR/Cas9 targeted at least two mutant sperm alleles despite injection into the zygote. Without being bound by theory, two different possibilities may explain this outcome: 1) at the time of injection, a zygote completed the S-phase of the cell cycle with DNA replication and already produced two mutant alleles, or 2) CRISPR/Cas9 remained active after introduction continuing to target after zygotic division (Capmany et al., Mol Hum Reprod, 2:299-306, 1996).

Both situations could be abrogated if CRISPR/Cas9 was co-injected together with sperm into the M-phase oocyte during intracytoplasmic sperm injection (ICSI) fertilization, allowing genome editing to occur when the sperm undoubtedly still contains a single mutant copy. In addition, the extended time of exposure to MII cytoplasm could allow CRISPR/Cas9 components to degrade before DNA replication results in two or more mutant alleles (FIG. 3A). Therefore, CRISPR/Cas9 was mixed with a sperm suspension and co-injected into 75 MII oocytes during the ICSI procedure with no difference observed in the survival, fertilization, and cleavage rates between CRISPR/Cas9 injected and intact control oocytes. At day 3 after fertilization, embryos at the 4-8-cell stage were disaggregated, and each individual blastomere was analyzed as described above for S-phase injected zygotes. Blastomeres from 16 of 58 M-phase injected embryos (27.6%) were uniformly heterozygous, carrying an intact WT maternal allele along with NHEJ-repaired mutant paternal sequences carrying various indels (MYBPC3^{WT/ΔGAGT-indel}) in every cell (FIG. 3B). The remaining 42 (72.4%) were MYBPC3^{WT/WT}. Of these, the vast majority (41/42) were uniformly homozygous embryos, consisting of blastomeres carrying indistinguishable MYBPC3^{WT/WT} alleles. Interestingly, the remaining embryo M2-WT42 contained 4 blastomeres with MYBPC3^{WT/WT} but HDR-repaired with ssODN, while the other 3 sister blastomeres were indistinguishable MYBPC3^{WT/WT}, demonstrating HDR using the maternal WT allele. No heterozygous blastomeres with intact mutant alleles (MYBPC3^{WT/ΔGAGT}) were detected, indicating 100% targeting efficiency in the M-phase injected group compared to 72.2% efficiency in the S-phase injected zygotes (FIG. 2D and FIG. 3B). More importantly, all sister blastomeres in all but one embryo carried identical genotypes, indicating a dramatic reduction in mosaicism in M-phase injected embryos. The only mosaic embryo had all blastomeres repaired by HDR (either WT or ssODN as a template). Thus, this embryo with every blastomere carrying repaired MYBPC3^{WT/WT} would be eligible for transfer.

The yield of MYBPC3^{WT/WT} embryos (72.4%, 42/58) in the M-phase injected group compared to untreated controls (47.4%, 9/19) was significantly higher (FIG. 3C, P<0.05), reflecting enhanced targeted correction of the mutant paternal alleles with DSB repair using the WT homologous chromosome as a template even in the presence of ssODNs (FIG. 3D). To rule out the possibility that the observed increase in WT/WT embryos in CRISPR-Cas9-injected zygotes and oocytes was due to allele drop-out during PCR and Sanger sequencing, genotypes were validated by independent on-target deep sequencing. Estimated HDR-based repair and increase of WT/WT embryos for S-phase and M-phase injected groups were 16.7% (9/54) and 22.4% (13/58), respectively (FIG. 3E). In summary, delivery of CRISPR/Cas9 into MII oocytes provides more efficient targeting and eliminates mosaicism compared with zygote injection.

### Example 5

This example describes development and cytogenetics of repaired embryos.

To examine the effect of gene correction on preimplantation development, CRISPR-Cas9-injected embryos were cultured to blastocysts. Similar to the intact controls, 72.7% (16/22) of M-phase-injected embryos developed to the 8-cell stage, and 50.0% (11/22) progressed to blastocysts (Student's t-test, P>0.05; FIG. 10A-10B). Cell lines were established to provide additional insights into the developmental competence of gene-corrected blastocysts and to obtain sufficient cellular material for detailed cytogenetic studies, including six ES cell lines from CRISPR-Cas9-injected blastocysts and one from controls. On-target analyses revealed that four CRISPR-Cas9-treated ES cell lines (ES-WT1, ES-WT2, ES-WT3, and ES-WT4) and one control cell line (ES-C1) were MYBPC3^{WT/WT}, whereas the remaining two CRISPR-Cas9-injected cell lines (ES-Mut1and ES-Mut2) were MYBPC3^{WT/ΔGAGT-indel}. These results corroborate the exceptionally high targeting efficiency of CRISPR-Cas9 in M-phase-injected human embryos.

Cytogenetic G-banding analysis revealed that ES-WT1, ES-WT4, ES-Mut1, and ES-Mut2 carried normal diploid karyotypes with no evidence of detectable numerical or structural chromosomal rearrangements. Notably, ES-WT2, ES-WT3, and the control line ES-C1 exhibited a pericentric inversion on chromosome 10. Thus, because both treated and control ES cells showed this chromosomal rearrangement, the rearrangement was contributed by the sperm and can be inherited. An analysis of the patient's skin fibroblast-derived iPSCs showed the same inversion, indicating that this inversion was balanced. In summary, CRISPR-Cas9-treated human embryos displayed normal development to blastocysts and ES cells without cytogenetic abnormalities.

### Example 6

### Potential Off-Target Consequences in CRISPR/Cas9 Injected Human Embryos

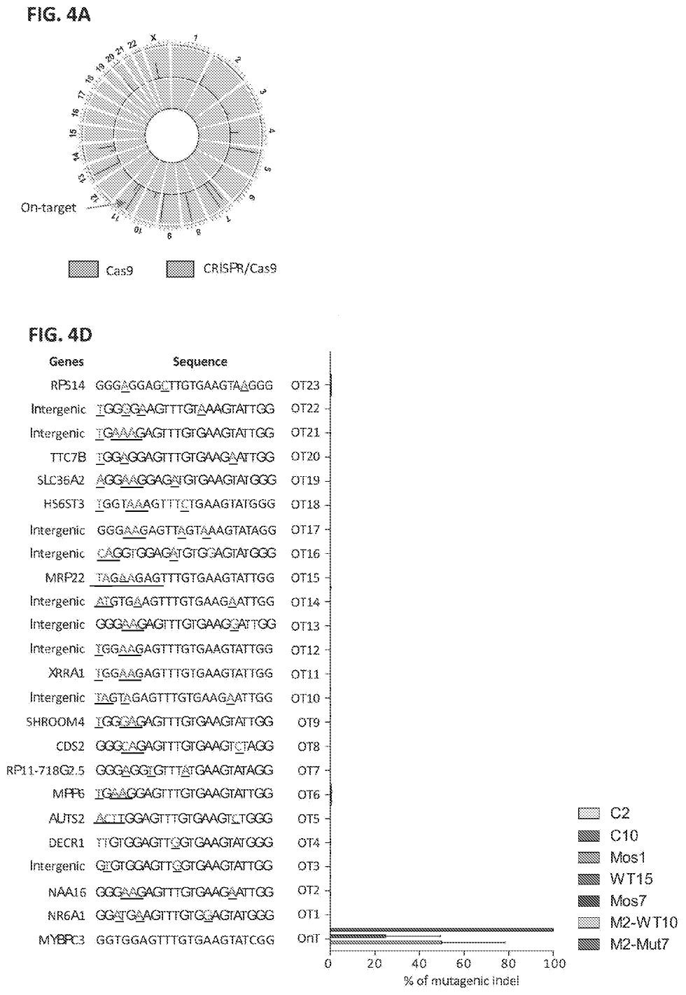

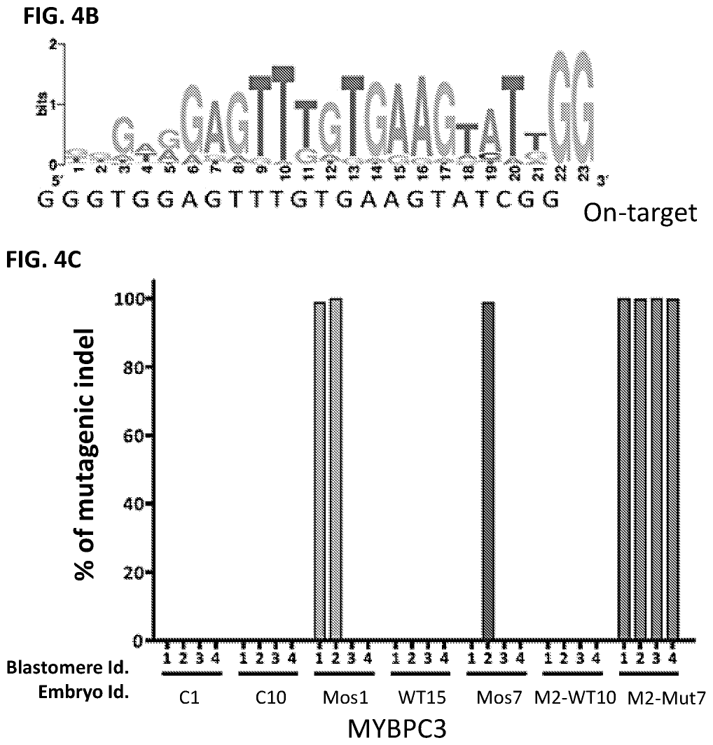

In addition to the overall targeting and HDR efficacy and mosaicism, one of the safety concerns regarding clinical application of gene correction in human embryos is that CRISPR/Cas9 can induce undesirable off-target mutations at genome regions highly homologous to the targeted sequence (Hsu et al., 2014; Mali et al., 2013; Fu et al., Nature biotechnology, 31:822-826, 2013; Hsu et al., Nature biotechnology, 31:827-832, 2013; Cho et al., Genome research, 24:132-141, 2014). Therefore, a comprehensive, whole genome sequencing (WSG) analysis of the patient's genomic DNA was conducted using a digested genome sequencing (Digenome-seq) approach (Kim et al., 2015; Kim et al., 2016). Potential off-target sequences were identified by digestion of iPSC-derived, cell-free genomic DNA with CRISPR/Cas9 followed by WGS. Sequencing reads of CRISPR/Cas9-digested genomic DNA are vertically aligned at on-/off-target sites in IGV viewer (Kiln et al., 2015; Robinson et al., Nature biotechnology, 29:24-26, 2011). In contrast, undigested genomic sites are aligned in a staggered manner in those loci. In addition, improved Digenome-seq provides DNA cleavage scores for potential off-target sites based on alignment patterns of sequence reads (Kim et al., 2016). Digested iPSC DNA produced uniform cleavage patterns in both on-target and potential off-target sites (FIGS. 6A-6B). In this analysis, 16 potential off-target sites were identified with a DNA cleavage score higher than 2.5 (FIG. 4A). A sequencing analysis of these 16 sites with Web Logo (see weblogo.berkeley.edu) confirmed that they are indeed highly homologous to the on-target MYBPC3 mutant allele (FIG. 4B; Kim et al., 2016; Schneider and Stephens, Nucleic acids research, 18:6097-6100, 1990). Furthermore, 7 additional sites were identified with DNA cleavage scores of 0.1 or greater and with 10 or fewer nucleotides mismatched in the human genome. Next, all of these sites were sequenced and analyzed in each individual blastomere from the two untreated control embryos (C2 and C10 from Supplementary Table 2); two mosaic S-phase injected embryos (Mos1 and Mos7); one uniform, non-mosaic S-phase injected embryo (WT15); and two M-phase injected embryos (M2-WT10 and M2-Mut7). All on-target indels in each blastomere were corroborated, and the results were identical to the Sanger sequencing results. In addition, indels were not detected in any blastomeres known to be carrying either intact WT/WT or WT/Mut alleles at the target site (FIG. 4C). More importantly, indels were also not detected in 23 off-target loci examined in 28 screened blastomeres (FIG. 4D).

In selected blastomeres (FIG. 4C), extended off-target screening was extended to WGS. Potential off-target sites were examined by comparing genomic variants found in intact control embryos with those in CRISPR-Cas9-injected embryos (Mos1.1, W15.4, Mos7.2, M2-WT10.1, and M2-Mut7.1). After filtering out annotated variants in the dbSNP database, 19-71 potential off-target sites with indels were found in each blastomere obtained from CRISPR-Cas9-injected embryos. All of these sites contained repeated sequences, such as poly-A or poly-GT repeats, suggesting that the indels found at these sites were caused by sequencing errors rather than Cas9-catalyzed, off-target DNA cleavage. These WGS results support the Digenome-seq data, wherein gene correction did not induce detectable off-target mutations in selected blastomeres.

Whether CRISPR-Cas9 targeting induced global off-target genetic variations and genome instability was assayed using whole-exome sequencing (WES) in CRISPR-Cas9-treated ES cells and comparing the results to those of control ES cells and corresponding egg and sperm donor blood DNA. WES analysis revealed a large number of variants in all samples when compared to the hg19 reference genome. The majority of these variants were also present in egg or sperm donors and found in the dbSNP and 1000 genomes databases. Some variants detected in ES cells showed decreased fractions matching the population hotspots, indicating the potential effect of experimental procedures, including embryo culture and ES cell derivation and culture. Three treated ES cell lines and a control line (ES-Mut1, ES-WT1, ES-WT2, and ES-C1) showed similar statistics in all variant categories and were comparable to gamete donor profiles (egg donors 1 and 2, sperm donor). ES-WT3 exhibited an increase in variant numbers, but this sample did not have a control sibling ES cell line for comparison. Next, potential off-target effects in ES cells were investigated, and a total of 685 potential off-target sites were identified using full sensitive aligner Batmis (V3.00; Tennakoon, Bioinformatics, 28:2122-2128, 2012). Variants that were also present in the gamete donors were filtered out as inherited. Notably, an analysis of these sites did not reveal any variants. Taken together, these Digenome-seq, WGS, and WES results demonstrate high on-targeting specificity of CRISPR-Cas9 in human embryos without any off-target effects.

DSBs induced by genome editing are primarily resolved via error-prone NHEJ, and such repair approaches are predominantly used to generate gene knockouts in cells and organisms (Richardson et al., Nature biotechnology, 34:339-344, 2016; Doudna and Charpentier, Science, 346, 2014). In contrast, HDR, although occurring at substantially lower efficiency, is necessary for gene correction, particularly for applications in human germline gene therapy. It was discovered that Cas9-mediated DSBs in human gametes and zygotes were preferentially resolved using an endogenous HDR mechanism that is exclusively directed by the wild-type allele as a repair template. In contrast, HDR efficiency in iPSCs was significantly lower and primarily achieved through an exogenous DNA template. This striking difference shows that human gametes/embryos employ a different DNA damage response system, perhaps reflecting the evolutionary significance of maintaining germline genome integrity (Luo et al., PLoS genetics, 10:e1004471, 2014). Gametes and zygotes that endure an increased number of DSBs during meiotic recombination and segregation have an efficient genome repair capacity, and unique zygotic DNA repair machinery might rely entirely on maternal oocyte factors deposited and stored during maturation since zygotes are transcriptionally silent (Lange et al., Cell, 167:695-708, e616, 2016). Recent studies show that oocytes might employ an ataxia-telangiectasia mutated (ATM)-mediated DNA damage signaling (DDS) pathway that regulates repair of DSBs via a homologous recombination mechanism (Titus et al., Sci Transl Med, 5:172ra121, 2013). Thus, in the studies disclosed herein, Cas9-induced DNA breaks are recruit the existing native oocyte machinery reserved for repair of meiotic recombination induced DSBs. Thus, it is not necessary to provide exogenous oligo templates for gene correction in heterozygous human embryos.

CRISPR/Cas9 efficacy was recently evaluated in a mouse study involving a heterozygous dominant mutation in the Crygc gene responsible for an inherited form of cataracts. While some HDR-repaired events utilized sequences from the WT allele from the homologous chromosome, some HDR occurred via an exogenous oligo template at a greater frequency with 3 of 4 pups carrying corrected Crygc genes with a DNA sequence from the exogenous oligo and only one from the WT allele (Wu et al., 2013). In a study involving human heterozygous embryos, HDR was exclusively directed by the exogenous DNA template with no evidence of WT allele-based repair (Tang et al., Mol Genet Genomics, 292(3):525-533, 2017). Because these results were derived using bulk DNA from whole embryos rather than individual blastomeres, cases of HDR via the WT allele could be overlooked.

Despite remarkable targeting efficiency and high HDR frequency, a portion of CRISPR/Cas9-treated human embryos exhibited NHEJ-induced indels and, thus, would not be suitable for transfer. Therefore, genome editing approaches must be further optimized before proceeding to clinical applications of germline correction. Modifications in genome editing by inhibition of the NHEJ mechanisms while enhancing HDR pathways have been reported (Chu et al., Nat Biotechnol, 33:543-548, 2015; Maruyama et al., Nat Biotechnol, 33:538-542, 2015). Other approaches have focused on manipulating the cell cycle or modifying the donor ssDNA design (Lin et al., 2014; Richardson et al., 2016). While some of these developments significantly improved HDR outcomes in the context of cultured cells, their relevance to embryonic gene correction remains unknown. In addition, supplementary exposure of human gametes or embryos to small molecules and/or inhibitors may be undesirable secondary to potentially adverse effects on embryonic development.

Non-human primate studies demonstrate that CRISPR/Cas9 injection into monkey zygotes can disrupt WT genes with the resultant full term offspring carrying the mutations and associated phenotypes (Niu et al., Cell, 156:836-843, 2014; Kang et al., Human molecular genetics, 24:7255-7264, 2015). Similar to outcomes seen in mice and other animals, genome-edited human preimplantation embryos and newborn monkeys display mosaic targeting genotypes in their cells and tissues, demonstrating that DSBs and subsequent repair does not occur at the single mutant allele stage (Tang et al., 2017; Liang et al., Protein & cell, 6:363-372, 2015; Tu et al., Sci Rep, 7:42081, 2017). As discussed above, mosaicism in gene corrected human embryos is difficult to detect by biopsy and PGD, thus, poses serious safety issues for clinical applications. Modifications involving shortening the half-life of Cas9 activity reduced, but did not completely eliminate, the manifestations of mosaicism in monkey embryos (Tu et al., 2017).

Further, the delivery of CRISPR/Cas9 into M-phase oocytes abolished mosaicism in cleaving embryos, demonstrating that the gene targeting and editing efficiencies are strongly associated with DNA synthesis and the cell cycle phase. Without being bound by theory, the choice of DSB repair using either NHEJ or HDR may depend on cell cycle phase with HDR restricted to late S and G2 phases when DNA replication is completed and sister chromatids are available as repair templates (Lin et al., 2014). Particularly, HDR mechanisms were down-regulated at the M and early G1 phases, thus favoring NHEJ-induced genome editing (Orthwein et al., Science, 344:189-193, 2014). However, reduced HDR efficiency was not observed even when CRISPR/Cas9 was delivered into Mil oocytes at the time of ICSI. Without being bound by theory, one explanation is that the DNA repair response is different in germ cell meiotic M phase compared with mitotic M phase in cultured cells. Alternatively, the DSBs may have occurred at the M or G1 phase, while the HDR repair followed later at the S or G2 phase of the cell cycle.

Extensive reports on potential off-target DNA damage induced by CRISPR/Cas9 beyond the intended targeting region have been published. In particular, Cas9 overexpression via plasmid transfection and subsequent high enzyme concentrations were reported to increase off-site targeting (Kim et al., 2014). In human oocytes and zygotes, purified recombinant Cas9 protein was used instead of plasmid, which, without being bound by theory, may have enhanced the specificity while shortening enzymatic exposure time, thereby diminishing off-targeting effects. Screening by Digenome-seq did not show off-target mutations in multiple individual blastomeres from CRISPR/Cas9-injected human embryos. These results indicate that CRISPR/Cas9 targeting is accurate, providing assurance for safety concerns related to gene correction in human embryos.

PGD may be a viable option for heterozygous couples at risk for producing affected offspring. In cases where only one parent carries a heterozygous mutation, 50% of embryos should be mutated and would be discarded. The methods and compositions disclosed herein demonstrate that targeted gene correction can rescue a substantial portion of mutant human embryos, thus increasing the number of embryos available for transfer.

### Examples 7-12

Examples 7-12 show that human embryos have the capacity for non-meiotic homologous chromosome-based DNA repair. Mounting evidence suggests that two parental homologs provide more than a genetic diversity contributed by parents (Joyce et al., Current opinion in genetics & development, 37:119-128, 2016). Recent developments in custom-designed nucleases, allowing for selective targeting of one of the two parental alleles, show inter-chromosomal pairing, interaction, and contribution to DNA repair across plant and animal species. Such interactions include DNA DSB repair governed by mitotic recombination or homolog-template-based repair contributing to LOH (Rong and Golic, Genetics, 165:1831-1842, 2003). One study utilizing mutant tomato plants with different fruit colors concluded that, in heterozygous plants, CRISPR-Cas9-induced DSBs in the targeted allele were repaired using the intact allele as a template at a frequency of up to 14% and that HDR between homologs occurred in the absence of the meiotic machinery (Filler Hayut et al., Nature communications, 8:15605, 2017). Specific targeting of the mutant paternal allele in heterozygous mice also demonstrated that DSB repair via HDR using the WT maternal allele resulted in the birth of viable WT/WT offspring (Wu et al., 2013). Simultaneous DSB induction in both parental alleles could also induce template-mediated repair using endogenous genomic sequences from close homologous gene families. In human zygotes, CRISPR-Cas9-based bi-allelic targeting of the β-globin gene (HBB) resulted in HDR using the endogenous delta-globin gene (HBD) (Liang et al., 2015).

### Example 7

This example describes the methods and materials used in Examples 7-12.

### Long-Range PCR and Sanger Sequencing

Long-range PCR (PCR1, PCR2, and PCR4-7) was performed using PrimeSTAR GXL DNA Polymerase, while the long-range PCR3 and PCR8 was performed with TaKaRa LA Taq DNA Polymerase (Clontech), according to manufacturer's procedure. In brief, PCR conditions were 10 sec at 98° C., 15 sec at 60° C., and 1 min/kb at 68° C. (30-35 cycles). PCR products were resolved with 1% agarose gel electrophoresis and were visualized with EtBr staining.

For Sanger sequencing, targeted region PCR for each single nucleotide polymorphisms (SNPs) was carried out using the PCR Platinum SuperMix High Fidelity Kit (Life Technologies). The PCR products were Sanger-sequenced and analyzed by Sequencher v5.0 (GeneCodes).

### Parentage Analysis by Short Tandem Repeat (STR) Assay

DNA was extracted from blood of egg and sperm donors and individual ESC lines using commercial kits (Gentra). STR microsatellite parentage analysis was conducted by the Genetics Laboratory at University of California, Davis, as described (Tachibana et al., 2013).

### SNP Searching and Calling Using Whole-Exome Sequencing (WES) and Whole-Genome Sequencing (WGS)

WES sequencing data were first processed by filtering adaptor sequences and removing low quality reads or reads with a high percentage of N bases using SOAPnuke (1.5.2) software (see soap.genomics.org.cn) developed by BGI. Clean reads were generated for each library. Clean data were paired-end aligned using the Burrows-Wheeler Aligner14 (BWA) program version 0.7.12 to the human genome assembly hg19. Duplicate reads in alignment BAM files were identified using MarkDuplicates in Picard v1.54 (see broadinstitute.github.io/picard). The alignment results were processed by RealignerTargetCreator, IndelRealigner, and BaseRecalibrator modules in GATK15 (3.3.0), and variant detection was performed using the HaplotypeCaller tool in GATK, according to GATK Best Practices recommendations (Van der Auwera et al., Current protocols in bioinformatics, 43:11.10.11-33, 2013; DePristo et al., Nature genetics, 43:491-498, 2011). SNV and InDel information were extracted and filtered by VQSR in GATK as well as annotated by AnnoDB v3 (see igm.columbia.edu).

### Example 8

This example describes assays for large deletions at the targeted region induced by CRISPR-Cas9. Without being bound by theory, repair of the mutant paternal allele using maternal-homologous sequences is unlikely because, in early zygotes, parental genomes are physically separated in paternal and maternal pronuclei. This temporary isolation precludes the homologous chromosome interactions required for HDR. However, CRISPR-Cas9 ribonucleoprotein (RNP) specific to the mutant paternal allele was delivered into pronuclear stage zygotes or even earlier during fertilization in Examples 1-6, while subsequent readouts of targeting and repair outcomes were measured three days later in multicellular embryos (Antoniou et al., Am J Hum Genet, 72:1117-1130, 2003). In late mammalian zygotes, paternal and maternal pronuclei migrate toward each other with subsequent nuclear envelope breakdown and formation of a diploid mitotic spindle (Capmany et al., 1996; Lemmen et al., Reprod Biomed Online, 17:385-391, 2008). Thus, from that point on, parental homologs are presented with opportunities to physically interact and recombine. Further, each mosaic 4-8-cell embryo contained blastomeres with two or more different repair outcomes, showing that CRISPR-Cas9 remains active well beyond the pronuclear stage.

To assay for any large deletions (>100 bp) induced by CRISPR-Cas9 at the targeted region, particularly, when disrupting both parental alleles simultaneously, different sgRNAs in patient iPSCs were designed, and a sgRNA was selected with high specificity for the mutant sequence and with no evidence of large deletions. Thus, the selected sgRNA would not induce large deletions at the frequency observed for HDR. A previous study showed that testing multiple candidate sgRNAs in human ES cells was effective for predicting editing efficacy of disrupting both copies of POU5F1 in human embryos (Fogarty et al., Nature, 550:67-73, 2017). In the study, the most frequently observed on-target editing in CRISPR-Cas9-microinjected human embryos included small (2-3 bp) indels. Only one embryo contained a few blastomeres with uncommonly large (330 bp) deletions (id.). While species differences may have impacted editing outcomes, Adikusuma et al. did not report pre-testing candidate sgRNAs. Moreover, the examples herein employ CRISPR-Cas9 ribonucleoprotein (RNP), while Adikusuma et al. employed Cas9 mRNA, which may have accounted for large deletions.

This example describes large-scale re-testing of all embryonic blastomere samples from Examples 1-6. In Examples 1-6, PCR amplification was employed followed by Sanger sequencing of a 534-bp fragment, spanning approximately 250 bp in each direction from the MYBPC3^{ΔGAGT} mutation site. In this example, an additional 8 pairs of long-range PCR primers were designed to amplify various lengths of fragments surrounding the MYBPC3ΔGAGT mutation locus, ranging from 493 bp to 10,160 bp (PCR1-PCR8 in FIG. 7A). First, 8 blastomeres were re-tested with WT/WT genotypes from the 4 mosaic embryos S-phase injected with CRISPR-Cas9 along with 4 WT/WT and 4 WT/Mut blastomeres from the non-injected control embryos. PCR products were separated on 1% agarose gels. In all 16 samples, primers PCR1, PCR2, PCR4, and PCRS amplified a single band of the expected size (FIGS. 7B-7E). For PCR6 and PCR7, several faint bands of smaller size were also detectable in some corrected and control blastomeres (FIGS. 7F and 7G). However, Sanger sequencing of these faint bands did not produce readable products, demonstrating non-specific PCR primer binding. Next, two additional long-range PCR amplifications were performed with PCR3 and PCR8 primers on all remaining WT/WT blastomeres (n=35) from the 13 mosaic embryos along with controls. Amplification with PCR3 produced a single band of the expected 1,742 bp size in all experimental and control blastomeres (FIG. 7H). For PCR8, in addition to a major band matching the expected 10,160 bp size, a few faint smaller size bands were also visible in some targeted and control samples, but, again, Sanger sequencing showed non-specific primer binding (FIG. 7I).

Larger deletions in the M-phase injected embryos were screened, and one blastomere from every WT/WT embryo (n=41) was randomly selected because all individual blastomeres within each embryo in this group carry identical MYBPC3 genotypes. The only mosaic embryo (M2-WT42) in this group was also tested, which contained 3 blastomeres with WT/WT genotypes and 4 blastomeres with WT/ssODN. Again, long-range PCR screening of all samples with primers PCR3 and PCR8 produced a single band of the expected 1,742 bp or 10,160 bp size (i.e., failing to detect large deletions; FIGS. 7J and 7K).

Whole-exome sequencing (WES) results for large deletions in the 6 human ES cell lines derived from M-phase injected embryos were also examined Comparisons of the area 5 kbp downstream and 5 kbp upstream of the mutation site in ES cells and the corresponding egg and sperm donors revealed no differences in sequencing depth, consistent with an absence of large deletions.

In summary, all of the results in this example demonstrate a failure to detect the presence of large deletions up to ±5 kbp from the mutation site in CRISPR-Cas9-treated human embryos. PCR primers employed in this example did not identify larger deletions, and deletions induced by CRISPR-Cas9 should have been detected with our assays. Utilization of multiple sgRNAs targeting several sites may produce large deletions of up to 24-kbp DNA segments; however, single sgRNA results in smaller deletions of <600-bp DNA in mouse embryos (Shin et al., Nature communications, 8:15464, 2017).

### Example 9

Examples 1-6 show that DSB repair on the paternal allele governed by maternal homolog-based HDR extends to the adjacent ΔGAGT deletion site, resulting in conversion of the paternal sequence. Therefore, whether DNA proofreading and mismatch repair mechanisms involved in HDR could also contribute to the conversion of neighboring neutral paternal SNPs resulting in loss-of-heterozygosity (LOH) within the MYBPC3 locus was assayed. Paternal SNPs adjacent to the targeted DSB locus converted to become maternal-like, while more distant polymorphic sites are preserved. Whole-exome sequencing (WES) and whole-genome sequencing (WGS) datasets were searched, and three informative parental SNPs were identified within the MYBPC3 gene, distinguishing egg donor 1 from the sperm donor. SNPs#1 (rs2071304) and #2 (rs2856650) were located downstream of the ΔGAGT deletion site (−7,959 bp and −781 bp), while SNP#3 (rs2856653) was +3,335 bp upstream from this locus (FIG. 8A). Next, individual blastomeres of the two CRISPR-Cas9 injected mosaic embryos (Mos2 and Mos3 in Table 1) from this parental combination were genotyped. An ES cell line (ES-C1) derived from the control non-injected blastocyst from the same parental combination was also genotyped. ES-C1 with a WT/WT genotype at the mutation locus and two blastomeres, Mos2.3 and Mos3.2, from the mosaic embryos with WT/NHEJ genotype were heterozygous at all 3 polymorphic sites, representing the expected maternal and paternal SNPs (G/C, T/C, and G/A for SNPs#1, #2, and #3, respectively) (Table 1 and FIG. 8B). In contrast, two blastomeres, Mos2.1 and Mos3.1, with WT/HDR genotypes from the same mosaic embryos were homozygous for all 3 SNP sites, carrying exclusively maternal nucleotides. Interestingly, another blastomere, Mos2.2, also with a WT/HDR genotype was homozygous at the SNP#2 locus, carrying maternal nucleotides, but heterozygous at both SNP#1 (G/C) and SNP#3 (G/A), demonstrating preservation of these paternal SNP sites (Table 1 and FIG. 8B). These results show that HDR-based conversion can expand beyond the targeted mutant loci, resulting in a loss of neutral paternal SNPs across the MYBPC3 region.

TABLE 1
MYBPC3 SNP genotypes in individual blastomeres of S-phase- and M-phase-
injected embryos derived from egg donor 1 and MYBPC3^{ΔGAGT} sperm donor

| | | | MYBPC3 | | | |
|---|---|---|---|---|---|---|
| Treatment | Samples | Blastomere ID | genotype | SNP#1 −7959* | SNP#2 −781* | SNP#3 +3335* |
| | Egg donor 1 | N/A | WT/WT | G/G | T/T | G/G |
| | Sperm donor | N/A | WT/Mut | C/C | C/C | A/A |
| Control | ES-C1 | N/A | WT/WT | G/C | T/C | G/A |
| S-phase | Mos2 | Mos2.3 | WT/NHEJ | G/C | T/C | G/A |
| injected | | Mos2.1 | WT/HDR | G/G | T/T | G/G |
| | | Mos2.2 | WT/HDR | G/C | T/T | G/A |
| | Mos3 | Mos3.2 | WT/NHEJ | G/C | T/C | G/A |
| | | Mos3.1 | WT/HDR | G/G | T/T | G/G |
| S-phase | WT3 | WT3.3 | WT/WT | G/C | T/T | G/A |
| injected | | WT3.4 | WT/WT | G/C | T/T | G/A |
| | WT4 | WT4.1 | WT/WT | G/C | T/C | G/A |
| | | WT4.4 | WT/WT | G/C | T/C | G/A |
| | WT5 | WT5.1 | WT/WT | G/C | T/C | G/A |
| | | WT5.2 | WT/WT | G/C | T/C | G/A |
| | WT6 | WT6.1 | WT/WT | G/C | T/C | G/A |
| | | WT6.2 | WT/WT | G/C | T/C | G/A |
| M-phase | M2-WT29 | M2-WT29.2 | WT/WT | G/C | T/C | G/A |
| injected | | M2-WT29.3 | WT/WT | G/G | T/T | G/G |
| | M2-WT30 | M2-WT30.2 | WT/WT | G/G | T/T | G/G |
| | | M2-WT30.3 | WT/WT | G/C | T/C | G/A |
| | M2-WT31 | M2-WT31.1 | WT/WT | G/C | T/T | G/G |
| | | M2-WT31.2 | WT/WT | G/C | T/C | G/A |
| | M2-WT32 | M2-WT32.2 | WT/WT | G/C | T/C | G/A |
| | | M2-WT32.3 | WT/WT | G/G | T/T | G/G |
| | M2-WT28 | M2-WT28.1 | WT/WT | G/C | T/C | G/A |
| | | M2-WT28.5 | WT/WT | G/C | T/C | G/A |
| | M2-WT33 | M2-WT33.2 | WT/WT | G/C | T/C | G/A |
| | | M2-WT33.3 | WT/WT | G/C | T/C | G/A |

*Represents downstream and upstream distance from the 4 bp deletion in the MYBPC3 gene. N/A, not applicable. Bold fonts indicate maternal nucleotides and underline fonts represent paternal nucleotides.

### Example 10

In contrast to the mosaic counterparts, the MYBPC3 genotype of original sperm in uniform WT/WT embryos produced from CRISPR-Cas9-treated zygotes or oocytes cannot be determined. Nevertheless, some embryos with MYBPC3^{WT/WT} genotypes can originate from mutant MYBPC3^{ΔGAGT} sperm subsequent HDR correction of the deletion because a significant increase in the percentage of WT/WT embryos in the CRISPR-Cas9-treated group was observed compared with non-treated controls (Antoniou et al., 2003). The loss of neutral paternal SNPs in some of these WT/WT embryos demonstrate repair of the mutant MYBPC3^{ΔGAGT}. Among 42 WT/WT M-phase-injected embryos, six (M2-WT28 through M2-WT33 in Table 1) were derived from egg donor 1 and the sperm donor and, thus, should be heterozygous at the SNP#1, #2, and #3 sites. Two sister blastomeres from each of these 6 embryos were randomly genotyped, and LOH was observed in at least one of these polymorphic sites in four embryos. As expected, paternal SNPs were lost at these loci, resulting in homozygous maternal nucleotides (Table 1 and FIG. 9). Genotypes of two sister blastomeres from the same embryo were distinct from each other, showing that independent HDR events occurred at the 2-cell stage or later. For example, one blastomere (M2-WT29.3) in the embryo M2-WT29 was homozygous at all 3 SNP loci, carrying exclusively maternal nucleotides, while the other sister blastomere (M2-WT29.2) was heterozygous at all 3 SNP sites (Table 1 and FIG. 9). A similar pattern was also observed in embryos M2-WT 30 and 32. In contrast, one blastomere (M2-WT31.1) of the embryo M2-WT31 was homozygous, containing maternal alleles at SNP#2 and #3 (T/T and G/G, respectively), while the more distant SNP#1 was heterozygous (G/C). The sister blastomere M2-WT31.2 was heterozygous at these 3 SNP positions.

As indicated above, LOH associated with erasure of paternal SNPs in these 4 uniform WT/WT embryos shows repair of the mutant sperm MYBPC3^{ΔGAGT} deletion following CRISPR-Cas9 treatment. All blastomeres examined in the remaining embryos, M2-WT28 and 33, were heterozygous at all 3 SNP sites, showing that these embryos were fertilized by WT sperm.

The SNP analysis was extended to four WT/WT embryos from the S-phase injected group of the same parental combination. Three embryos (WT4, WT5, and WT6) were heterozygous for all 3 SNPs, while both blastomeres examined from WT3 embryo were heterozygous at SNP#1 and #3 but homozygous at SNP#2 (Table 1 and FIG. 9). Thus, this embryo likely was generated from the mutant sperm but subsequently corrected by HDR using WT maternal allele.

### Example 11

To provide further genetic evidence for HDR, egg donor 2 was screened, and two informative SNPs were identified within the MYBPC3 gene that would differentiate from the paternal contribution. Egg donor 2 was homozygous (G/G) at the SNP#4 site (positioned −6,189 bp downstream of the ΔGAGT mutation, rs2697920), while the sperm donor was heterozygous (A/G) at this locus. At SNP#5 (+9,514 bp, rs4733354), both parents were heterozygous A/G. Initially, blastomeres with WT/NHEJ or WT/Mut genotypes from seven mosaic embryos (Mos1, Mos7, Mos8, Mos10, Mos11, Mos12, and Mos13) derived from this parental combination were genotyped, which showed that six were heterozygous A/G at the SNP#4 locus (italic font in Table 2), demonstrating that mutant sperm contributed the “A” allele at this locus in these embryos. All sister blastomeres with WT/HDR genotypes from these 6 embryos were sequence, and 5 (Mos1, Mos7, Mos8, Mos10, and Mos13) were identified as containing one or more blastomeres that lost the paternal allele and became homozygous G/G at the SNP#4, showing gene conversion from maternal allele (Table 2). The remaining WT/HDR blastomeres in these embryos retained the paternal allele and were heterozygous A/G at the SNP#4, demonstrating a shorter conversion tract. Both WT/HDR blastomeres from Mosll embryo were heterozygous A/G at the SNP#4. A WT/Mut blastomere from Mos12 embryo was homozygous G/G at the SNP#4; thus, further genotyping was unnecessary (Table 2).

Next, one randomly selected blastomere from each of the seven uniform WT/WT M-phase-injected embryos generated from the egg donor 2 and the sperm donor was sequenced. All seven blastomeres were homozygous G/G at the SNP#4 (Table 2). In comparison, 6 out of 7 S-phase-injected mosaic embryos generated from the same parental combination were heterozygous A/G (Table 2). Therefore, some of these G/G homozygous embryos in the M-phase-injected group also lost paternal SNPs due to gene conversion. Genotyping for the SNP#5 locus showed that 2 mosaic S-phase injected embryos (Mos1and Mos8) were heterozygous A/G and informative for conversion analyses (Table 2). All 5 sister blastomeres with WT/HDR genotypes in Mos1 embryo were homozygous G/G at the SNP#5, demonstrating a loss of paternal SNPs. As for two WT/HDR blastomeres in the Mos8 embryo, one was homozygous G/G, and one was heterozygous A/G at the SNP#5. Among the M-phase-injected embryos, 1 out of 7 was heterozygous A/G, while the remaining six were homozygous G/G at SNP#5 (Table 2).

These results show that gene conversion in human embryos induced by HDR occurs and extends significant distances in both directions from the original target site, resulting in LOH associated with erasure of neutral paternal SNPs. The length of the conversion tract varied among individual blastomeres, even from the same embryo. The existence of polymorphic sites and retention of paternal SNPs on some corrected blastomeres also shows that the mutant paternal MYPBC3 locus was repaired in S-phase- and M-phase-injected embryos.

TABLE 2
MYBPC3 SNP genotypes in individual blastomeres of S-phase- and M-phase-
injected embryos derived from egg donor 2 and MYBPC3^{ΔGAGT} sperm donor

| | | | MYBPC3 | | |
|---|---|---|---|---|---|
| Treatment | Samples | Blastomere ID | genotype | SNP#4 −6189* | SNP#5 +9514* |
| | Egg donor 2 | N/A | WT/WT | G/G | A/G |
| | Sperm donor | N/A | WT/Mut | A/G | A/G |
| S-phase | Mos1 | Mos1.1 | WT/NHEJ | A/G | A/G |
| injected | | Mos1.4 | WT/HDR | G/G | G/G |
| | | Mos1.5 | WT/HDR | G/G | G/G |
| | | Mos1.6 | WT/HDR | A/G | G/G |
| | | Mos1.7 | WT/HDR | G/G | G/G |
| | | Mos1.8 | WT/HDR | A/G | G/G |
| S-phase | Mos7 | Mos7.2 | WT/NHEJ | A/G | A/A |
| injected | | Mos7.1 | WT/HDR | A/G | N/A |
| | | Mos7.3 | WT/HDR | G/G | N/A |
| | | Mos7.4 | WT/HDR | A/G | N/A |
| S-phase | Mos8 | Mos8.3 | WT/Mut | A/G | A/G |
| injected | | Mos8.1 | WT/HDR | G/G | A/G |
| | | Mos8.2 | WT/HDR | A/G | G/G |
| S-phase | Mos10 | Mos10.5 | WT/Mut | A/G | A/A |
| injected | | Mos10.1 | WT/HDR | G/G | N/A |
| | | Mos10.2 | WT/HDR | A/G | N/A |
| | | Mos10.3 | WT/HDR | G/G | N/A |
| | | Mos10.4 | WT/HDR | G/G | N/A |
| S-phase | Mos11 | Mos11.3 | WT/NHEJ | A/G | A/A |
| injected | | Mos11.1 | WT/HDR | A/G | N/A |
| | | Mos11.2 | WT/HDR | A/G | N/A |
| S-phase | Mos13 | Mos13.3 | WT/Mut | A/G | A/A |
| injected | | Mos13.1 | WT/HDR | G/G | N/A |
| S-phase | Mos12 | Mos12.8 | WT/NHEJ | G/G | G/G |
| injected | | Mos12.1 | WT/HDR | G/G | N/A |
| | | Mos12.2 | WT/HDR | G/G | N/A |
| M-phase | M2-WT1 | M2-WT1.2 | WT/WT | G/G | A/G |
| injected | M2-WT2 | M2-WT2.2 | WT/WT | G/G | G/G |
| | M2-WT3 | M2-WT3.2 | WT/WT | G/G | G/G |
| | M2-WT4 | M2-WT4.3 | WT/WT | G/G | G/G |
| | M2-WT5 | M2-WT5.2 | WT/WT | G/G | G/G |
| | M2-WT6 | M2-WT6.2 | WT/WT | G/G | G/G |
| | M2-WT7 | M2-WT7.2 | WT/WT | G/G | G/G |

*Represents downstream and upstream distance from the 4 bp deletion in the MYBPC3 gene. N/A not applicable. Bold fonts depict maternal nucleotides and underline font shows paternal nucleotides. Italic font represents blastomeres with WT/Mut or WT/NHEJ MYBPC3 genotypes that were heterozygous A/G at SNP#4.

### Example 12

This example describes assaying for parthenogenetic development. Examples 1-6 show that early exposure to CRISPR-Cas9 RNP during fertilization (M-phase) significantly reduces or completely eliminates mosaicism in cleaving embryos. These results are irrespective of whether repair occurred via HDR or NHEJ because mosaic embryos may include blastomeres with different NHEJ-derived indel genotypes. Only one out of a total of 58 (1.7%) cleaving embryos produced by M-phase injection was mosaic, while 16 of 58 (27.6%) were uniformly heterozygous, carrying NHEJ-derived indels in the mutant paternal allele (MYBPC3^{WT/ΔGAGT-indel}; FIG. 3B) (Antoniou et al., 2003). These heterozygous embryos could not originate from parthenogenesis because they all carry the paternal MYBPC3 deletion. In contrast, when CRISPR-Cas9 was injected one day after fertilization into late S-phase zygotes, 13 of 54 (24%) embryos were mosaics (FIG. 2A; id.). Of 75 M-phase injected oocytes, two were lysed during ICSI and 10 failed to fertilize. The remaining 63 (84%) exhibited normal fertilization morphology with two pronuclei and two polar bodies, which is inconsistent with parthenogenic activation. Similar results were obtained from non-injected controls and S-phase-injected embryos (id.). Moreover, the SNP analyses provided in Tables 1 and 2 for WT/WT embryos in the M-phase injected group clearly demonstrate retention of paternal SNPs.

To assay for parthenogenetic development, the paternal contribution in WT/WT ES cell lines derived from M-phase-injected embryos was confirmed using a short tandem repeat (STR) assay. All 6 ES cell lines derived by M-phase injection and one non-injected control contained both maternal and paternal STR alleles. Thus, in all samples analyzed, paternal contribution was detected, and parthenogenesis was eliminated.

In view of the many possible embodiments to which the principles of the disclosure may be applied, it should be recognized that illustrated embodiments are only examples of the disclosure and should not be considered a limitation on the scope of the invention. Rather, the scope of the invention is defined by the following claims. We therefore claim as our invention all that comes within the scope and spirit of these claims.

1. A method for correcting a mutant allele of a gene of interest in a primate cell, comprising:
a) introducing a non-naturally occurring targeted nuclease and a site-specific nucleotide-binding guide that act together to introduce double-stranded breaks in the mutant allele into the primate cell, wherein:
i) the primate cell is undergoing mitotic cell division;
ii) the primate cell comprises a genome that is heterozygous for the mutant allele, such that the genome comprises one copy of the mutant allele and one copy of a wild-type allele;
iii) single-stranded oligonucleotides homologous to the wild-type allele are not introduced into the primate cell; and
b) allowing the primate cell to activate homology-directed repair of the double-stranded DNA breaks in the mutant allele,
thereby correcting the mutant allele using the normal wild-type allele as a repair template and producing a primate cell that is homozygous for the wild-type allele.

2. The method of claim 1, wherein the primate cell is an embryo.

3. The method of claim 2, further comprising generating the embryo prior to step (a).

4. The method of claim 2, wherein the embryo is a one-cell embryo.

5. The method of claim 2, further comprising:
selecting a primate oocyte comprising a genome having the mutant allele or the wild-type allele of the gene of interest;
fertilizing the primate oocyte with a sperm from the same primate species, wherein the sperm comprises the wild-type allele or the mutant allele of the gene of interest, respectively, thereby forming a one-cell primate embryo, wherein the primate embryo is heterozygous and comprises the one copy of the wild-type allele and the one copy of the mutant allele.

6. The method of claim 5, wherein the non-naturally occurring targeted nuclease and the site-specific nucleotide-binding guide are introduced into the primate oocyte simultaneously with fertilizing the primate oocyte.

7. The method of claim 5, wherein fertilizing the primate oocyte comprises intracytoplasmic sperm injection (ICSI).

8. The method of claim 5, wherein the primate oocyte is at metaphase II when the targeted nuclease and the site-specific nucleotide-binding guide are introduced.

9. The method of claim 2, further comprising culturing the embryo to form a multi-cell embryo in vitro.

10. The method of claim 9, wherein the multi-cell embryo is not mosaic for cells comprising the mutant allele.

11. The method of claim 1, wherein the targeted nuclease is clustered regularly interspaced short palindromic repeats (CRISPR)-associated (Cas)9, zinc finger nuclease (ZFN), or transcription activator-like effector nuclease (TALEN).

12-28. (canceled)

29. The method of claim 11, wherein the targeted nuclease is CRISPR-Cas9, and the site-specific nucleotide-binding guide is a nucleic acid guide RNA.

30. The method of claim 1, further comprising:
c) assaying for successful correction of the mutant allele.

31. The method of claim 30, wherein the assaying for successful correction comprises the use of Sanger sequencing.

32. The method of claim 1, further comprising assaying for off-target effects.

33. The method of claim 32, wherein the assaying for off-target effects comprises whole-genome sequencing.

34. The method of claim 1, wherein the primate cell is a somatic cell.

35. The method of claim 34, wherein the somatic cell is a mesoderm, endoderm, or ectoderm cell.

36. The method of claim 34, wherein the somatic cell is a cardiac cell, skin cell, white blood cell, liver cell, pancreatic cell, kidney cell, ovarian cell, testicular cell, prostatic cells breast cell, muscle cell, cell of the digestive system, cell of the respiratory system, or an osteogenic cell.

37. The method of claim 1, wherein the primate cell is a pluripotent or multipotent stem cell.

38. The method of claim 37, wherein the multipotent stem cell is a bone marrow stem cell, hematopoietic stem cell, mesenchymal stem cell, intestinal stem cell, neuronal stem cell, or dental stem cell.

39. The method of claim 1, wherein the primate is a human.

40. The method of claim 1, wherein the mutant allele comprises a) a deletion or an insertion as compared to the wild-type allele; b) a base pair substitution as compared to the wild-type allele; or c) a frame shift mutation as compared to the wild-type allele.

41. The method of claim 1, wherein the gene of interest is myosin binding protein C (MYBPC3), fibroblast growth factor receptor 3 (FGFR3), serpin family A member 1 (SERPINA1), protein kinase D1 (PKD), breast cancer 1 (BRCA1), breast cancer 2 (BRCA2), glycyl-tRNA synthetase (GARS), WNT signaling pathway regulator (APC), cystic fibrosis transmembrane conductance regulator (CFTR), chimerin 1 (CHN1), dystrophin (DMD), coagulation factor V (F5), fragil X mental retardation 1 (FMR1), glucosylceramidase beta (GBA), homeostatic iron regulator (HFE), coagulation factor IX (FIX), huntingtin (HD), fibrillin 1 (FBN1), dystrophia myotonica protein kinase (DMPK), cellular nucleic acid binding protein (CNBP), protein tyrosine phosphatase, non-receptor type 11 (PTPN11), Ras/Rac guanine nucleotide exchange factor 1 (SOS1), Raf proto-oncogene serine/threonine kinase (RAF1), Kras proto-oncogene GTPase (KRAS), collagen type alpha 1 chain (COL1A1), collagen type alpha 2 chain (COL1A2), synuclein alpha (SNCA), ubiquitin C-terminal hydrolase L1 (UCHL1), leucine rich repeat kinase 2 (LRRK2), Parkinson disease 3 (PARK3), parkin RBR E3 ubiquitin protein ligase (PARK2), parkinsonism associated deglycase (PARK7), PTEN induced putative kinase 1 (PARK6), apolipoprotein B (APOB), low density lipoprotein receptor (LDLR), low density lipoprotein receptor adaptor protein 1 (LDLRAP1), proprotein convertase subtilisin/kexin type 9 (PCSK9), actin alpha cardiac muscle 1 (ACTC1), actinin alpha2 (ACTN2), calreticulin 3 (CALR3), cysteine and glycine rich protein 3 (CSRP3), junctophilin2 (JPH2), myosin heavy chain 7 (MYH7), myosin light chain 2 (MYL2), myosin light chain 3 (MyL3), myozenin 2 (MYOZ2), nexilin F-actin binding protein (NEXN), phospholamban (PLN), protein kinase AMP-activated non-catalytic subunit gamma 2 (PRKAG2), titin-cap (TCAP), troponin I3 cardiac type (TNNI3), troponin T2 cardiac type (TNNT2), tropomyosin 1 (TPM1), titin (TTN), or vinculin (VCL).

42. The method of claim 36, wherein (a) the somatic cell is from a human subject that has breast cancer, (b) the somatic cell is a breast cell, and (c) wherein the gene of interest is BRCA1 or BRCA 2.

43. The method of claim 36, wherein:
(a) the somatic cell is from a human subject that has familial cardiomyopathy,
(b) the cell is a cardiac cell, and
(c) the gene of interest is MYBPC3, ACTC1, ACTN2, CALR3, CSRP3, JPH2, MYH7, MYL2, MyL3, MYOZ2, NEXN, PLN, PRKAG2, TCAP, TNNI3, TNNT2, TPM1, TTN, and/or VCL.

44. The method of claim 36, wherein:
(a) the somatic cell is from a human subject that has familial hypercholesterolemia,
(b) the cell is a cardiac cell, and,
(c) the gene of interest is APOB, LDLR, LDLRAP1, and/or PCSK9.


SEQUENCE LISTING (43 sequences)

SEQ ID NO: 1 - protein, 1368 aa, Streptococcus pyogenes [sequence omitted]

SEQ ID NO: 2 - DNA, 19 bp, Artificial Sequence
  example targeting sequence
ggtggagtttgtgaagtat

SEQ ID NO: 3 - DNA, 20 bp, Artificial Sequence
  example targeting sequence
gggtggagtttgtgaagtat

SEQ ID NO: 4 - DNA, 97 bp, Artificial Sequence
  example sgRNA sequence
ggtggagtttgtgaagtatguuuuagagcuagaaauagcuuaaaauaaggcuaguccguuaucaacuugaaaaaguggcaccgaucggugcuuuuuu

SEQ ID NO: 5 - DNA, 98 bp, Artificial Sequence
  example sgRNA sequence
gggtggagtttgtgaagtatguuuuagagcuagaaauagcuuaaaauaaggcuaguccguuaucaacuugaaaaaguggcaccgaucggugcuuuuuu

SEQ ID NO: 6 - DNA, 23 bp, Artificial Sequence
  on-target sequence
gggtggagtttgtgaagtatcgg

SEQ ID NO: 7 - DNA, 23 bp, Homo sapiens
gggaggagcttgtgaagtaaggg

SEQ ID NO: 8 - DNA, 23 bp, Homo sapiens
tggggaagtttgtaaagtattgg

SEQ ID NO: 9 - DNA, 23 bp, Homo sapiens
tgaaagagtttgtgaagtattgg

SEQ ID NO: 10 - DNA, 23 bp, Homo sapiens
tggaggagtttgtgaagaattgg

SEQ ID NO: 11 - DNA, 23 bp, Homo sapiens
aggaaggagatgtgaagtatggg

SEQ ID NO: 12 - DNA, 23 bp, Homo sapiens
tggtaaagtttctgaagtatggg

SEQ ID NO: 13 - DNA, 23 bp, Homo sapiens
gggaagagttagtaaagtatagg

SEQ ID NO: 14 - DNA, 23 bp, Homo sapiens
caggtggagatgtggagtatggg

SEQ ID NO: 15 - DNA, 23 bp, Homo sapiens
tagaagagtttgtgaagtattgg

SEQ ID NO: 16 - DNA, 23 bp, Homo sapiens
atgtgaagtttgtgaagaattgg

SEQ ID NO: 17 - DNA, 23 bp, Homo sapiens
gggaagagtttgtgaaggattgg

SEQ ID NO: 18 - DNA, 23 bp, Homo sapiens
tggaagagtttgtgaagtattgg

SEQ ID NO: 19 - DNA, 23 bp, Homo sapiens
tggaagagtttgtgaagtattgg

SEQ ID NO: 20 - DNA, 23 bp, Homo sapiens
tagtagagtttgtgaagaattgg

SEQ ID NO: 21 - DNA, 23 bp, Homo sapiens
tgggagagtttgtgaagtattgg

SEQ ID NO: 22 - DNA, 23 bp, Homo sapiens
gggcagagtttgtgaagtctagg

SEQ ID NO: 23 - DNA, 23 bp, Homo sapiens
gggaggtgtttatgaagtatagg

SEQ ID NO: 24 - DNA, 23 bp, Homo sapiens
tgaaggagtttgtgaagtattgg

SEQ ID NO: 25 - DNA, 23 bp, Homo sapiens
acttggagtttgtgaagtctggg

SEQ ID NO: 26 - DNA, 23 bp, Homo sapiens
ttgtggagttggtgaagtatggg

SEQ ID NO: 27 - DNA, 23 bp, Homo sapiens
gtgtggagttggtgaagtattgg

SEQ ID NO: 28 - DNA, 23 bp, Homo sapiens
gggaagagtttgtgaagaattgg

SEQ ID NO: 29 - DNA, 23 bp, Homo sapiens
ggatgaagtttgtggagtatggg

SEQ ID NO: 30 - DNA, 22 bp, Homo sapiens
ggtggagtttgtgaagtatcgg

SEQ ID NO: 31 - DNA, 33 bp, Homo sapiens
agcgggtggagtttgagtgtgaagtatcggagg

SEQ ID NO: 32 - DNA, 33 bp, Homo sapiens
  misc_feature (15)..(18): n is a, c, g, t or u
agcgggtggagtttnnnngtgaagtatcggagg

SEQ ID NO: 33 - DNA, 22 bp, Artificial Sequence
  ssODN-1
ggagtttgagtgcgaggtatcg

SEQ ID NO: 34 - DNA, 22 bp, Artificial Sequence
  ssODN-2
ggagttcgaatgtgaagtatcg

SEQ ID NO: 35 - DNA, 17 bp, Homo sapiens
tggagtttgagtgtgaa

SEQ ID NO: 36 - DNA, 11 bp, Homo sapiens
cgggtggagtt

SEQ ID NO: 37 - DNA, 17 bp, Homo sapiens
tggagtttgagtgtgaa

SEQ ID NO: 38 - DNA, 17 bp, Homo sapiens
tggagttcgaatgtgaa

SEQ ID NO: 39 - DNA, 17 bp, Homo sapiens
tggagtttgagtgtgaa

SEQ ID NO: 40 - DNA, 20 bp, Artificial Sequence
  Primer F
cccccacccaggtacatctt

SEQ ID NO: 41 - DNA, 20 bp, Artificial Sequence
  Primer R
ctagtgcacagtgcatagtg

SEQ ID NO: 42 - DNA, 13 bp, Artificial Sequence
  Adaptor sequence
agatcggaagagc

SEQ ID NO: 43 - DNA, 13 bp, Artificial Sequence
  Adaptor sequence
gctcttccgatct